(19) United States
(12) Patent Application Publication
(10) Pub. No.: US 2024/0124588 A1
(43) Pub. Date: Apr. 18, 2024

(54) COMPOSITIONS AND METHODS FOR DISABLING MYELOID CELLS EXPRESSING TREM1

(71) Applicant: PIONYR IMMUNOTHERAPEUTICS, INC., South San Francisco, CA (US)

(72) Inventors: Christopher CHAN, South San Francisco, CA (US); Caroline Gray HACKETT, San Francisco, CA (US); Venkataraman SRIRAM, Berkeley, CA (US); Mark Bryan HEADLEY, Berkeley, CA (US); Tiep Tu LE, Kensington, CA (US)

(21) Appl. No.: 18/361,780

(22) Filed: Jul. 28, 2023

Related U.S. Application Data
(63) Continuation of application No. 16/637,421, filed on Feb. 7, 2020, now abandoned.
(60) Provisional application No. 62/542,563, filed on Aug. 8, 2017.

(51) Int. Cl. C07K 16/28

(52) U.S. Cl. CPC C07K 16/2818; A61K 2039/507

(57) ABSTRACT

Provided herein are methods and compositions for enhancing an immune response and/or for the treatment of an immune-related condition in an individual, comprising disabling myeloid cells using an anti-Triggering Receptor Expressed on Myeloid Cells 1 (TREM1) antibody.

### 1. CROSS REFERENCE TO RELATED APPLICATIONS

This application is a Continuation of U.S. patent application Ser. No. 16/637,421, filed Feb. 7, 2020 which is a National Stage of International Application No. PCT/US2018/045680, filed Aug. 7, 2018, which claims the benefit of U.S. Provisional Application No. 62/542,563, filed Aug. 8, 2017, which is hereby incorporated in its entirety by reference for all purposes.

### 2. SEQUENCE LISTING

The instant application contains a Sequence Listing which has been submitted via Patent Center and is hereby incorporated by reference in its entirety. Said XML copy, created on Dec. 4, 2023, is named PII-006C1, and is 137,064 bytes in size.

### 3. BACKGROUND

Immunity plays a critical role in preventing tumor outgrowth. A complex microenvironment can develop within the lesion, and despite the recruitment of T-cells, there is often no effective control of the developing mass. Understanding the balance between tumor elimination and tumor escape may rely on a comprehension of the differential roles myeloid cells play in the tumor microenvironment.

Myeloid populations of the tumor microenvironment prominently include monocytes and neutrophils (sometimes loosely grouped as myeloid-derived suppressor cells), macrophages, and dendritic cells. Although intra-tumoral myeloid populations, as a whole, have long been considered non-stimulatory or suppressive, it has more recently been appreciated that not all tumor-infiltrating myeloid cells are functionally equivalent.

In normal tissues, many of these myeloid cells are essential for proper functioning of both innate and adaptive immunity and notably for wound repair. However in the setting of cancer, a significant excess of macrophages and dysfunctional or skewed populations of these and other cell types are commonly described. When considered as an aggregate population defined by single markers, such as CD68 or CD163, “macrophage” infiltration is correlated with worse outcomes in patients across multiple tumor types ((de Visser, Cancer Immunol Immunother, 2008; 57:1531-9); (Hanada et al., Int J Urol 2000; 7:263-9); (Yao et al. Clin Cancer Res, 520, 2001; 7:4021-6); (Ruffell et al., PNAS, 523 2012; 109:2796-801)). But the phenotypic and functional subsetting of macrophages from the tumor microenvironment is complicated by the similarity of macrophages and dendritic cells, and is problematic in tumor biology. A morphologic criterion has been often applied to the issue; one approach to try to differentiate dendritic cells from macrophages was based on a more spikey or dendritic morphology for the former and more veiled or bulbous morphology for the latter (Bell et al., J Exp Med 555, 1999; 190:1417-26). Other groups are trying to differentiate on the basis of genetic and cell-surface markers.

There is diversity in the antigen-presenting compartment within tumors, and T-cells can differentiate features of antigen-presenting cells (APC). Because T cells are a major driver of tumor immunity, understanding the exact features of their cognate APCs will be important. Myeloid cells are prominent among cells capable of presenting tumor-derived antigens to T-cells and thereby maintaining the latter in an activated state. Antigen presentation occurs within the tumor itself and likely influences the functions of tumor cytotoxic T-lymphocytes (CTLs). T-cell activation by antigen presenting cells (APC) is an important component in antigen-specific immune responses and tumor cell killing. As these myeloid populations represent major T-cell—interacting partners and antigen-presenting cells for incoming tumor-reactive cytotoxic T lymphocytes, understanding their distinctions may guide therapeutic avenues.

Triggering Receptor Expressed on Myeloid Cells 1 (TREM1, but also known as CD354, HGNC: 17760, Entrez Gene: 54210, UniProtKB: Q9NP99) belongs to the Ig superfamily of receptors and is highly expressed on subsets of myeloid cells including neutrophils, monocytes and macrophages. TREM1 lacks signaling motifs and instead, receptor activation is mediated through the adapter DAP12 (DNAX-activating protein 12) that leads to amplification of inflammatory responses (Bouchon, et al (2000) J. Immunol. 164 (10): 4991-4995). Specifically, crosslinking of TREM1 induces expression of IL-8, myeloperoxidase, TNFα and MCP-1. TREM1 expression is up-regulated on myeloid cells in response to Toll-Like Receptor stimulation (bacterial and fungi stimulation), and has been shown to contribute to, and amplify the acute inflammatory response during septic shock and infection (Cohen, (2001) Lancet. 358: 776-778). While the ligand for TREM1 has remained elusive, recently, PGLYRP1 (peptidoglycan recognition protein 1) has been identified as a potent ligand of TREM1 (Read et al, (2015) J of Immunol. 194: 1417-1421). In mice there are 5 activating forms of TREM receptors including TREM 1, 2, 3, 4, and 5, with a soluble form of TREM1 (sTREM1) released during infection. Mouse TREM1 and the human homolog TREM1 share relatively low sequence identity of 46% (Radaev, et al. (2003) Structure 11: 1527-1535). Structurally TREM1 consists of a single V-type immunoglobulin (Ig)-like domain (Ig-V) of about 130 amino acids, followed by a 70 amino acid neck region. In addition to the role TREM1 plays in sepsis, it has also been linked to inflammatory bowel disease. However very little is known about the role of TREM1 in the tumor microenvironment. (Schenk, et al (2007) JCI. 117: 3097-3106).

An unmet need exists for novel cancer therapeutic approaches that involve selectively decreasing the load of cells that are ineffective at stimulating T-cell responses, thereby enhancing the immune response within the tumor microenvironment.

All patents, patent applications, publications, documents, and articles cited herein are incorporated herein by reference in their entireties.

### 4. SUMMARY

In one aspect, provided herein are methods of killing, disabling, or depleting myeloid cells that express Triggering Receptor Expressed on Myeloid Cells 1 (TREM1) on the cell surface, comprising contacting the myeloid cells with an anti-TREM1 antibody or antigen-binding fragment thereof, wherein the antibody comprises an active Fc region and comprises at least one of: antibody-dependent cell-mediated cytotoxicity (ADCC) activity, complement-dependent cytotoxicity (CDC) activity, and antibody-mediated phagocytosis (ADCP) activity.

In any one of the embodiments herein, the active Fc region is wild-type afucosylated human IgG1 Fc that binds an activating Fcγ Receptor (FcγR) on an immune cell and kills, disables, or depletes the myeloid cells by at least one of ADCC, CDC, and ADCP, and wherein the antibody binds the extracellular domain of TREM1.

In any one of the embodiments herein, the active Fc region comprises human IgG1, IgG2, IgG3, or IgG4 Fc. In some embodiments, the active Fc region comprises wild-type, human IgG1 Fc. In any one of the embodiments herein, the Fc region binds an Fcγ Receptor selected from the group consisting of: FcγRI, FcγRIIa, FcγRIIb, FcγRIIc, FcγRIIIa, and FcγRIIIb.

In any one of the embodiments herein, the immune cell is a professional antigen presenting cell, a macrophage, a monocyte, a natural killer cell, a neutrophil, a dendritic cell, or a B cell.

In any one of the embodiments herein, the antibody kills, disables, or depletes the myeloid cells by at least one of ADCC, CDC, and ADCP, optionally wherein the antibody kills, disables, or depletes the myeloid cells by ADCC, optionally wherein the antibody kills, disables, or depletes the myeloid cells by CDC, and optionally wherein the antibody kills, disables, or depletes the myeloid cells by ADCP. In any one of the embodiments herein, the antibody kills the myeloid cells by at least one of ADCC, CDC, and ADCP. In any one of the embodiments herein, the antibody disables the myeloid cells by at least one of ADCC, CDC, and ADCP. In any one of the embodiments herein, the antibody depletes the myeloid cells by at least one of ADCC, CDC, and ADCP.

In any one of the embodiments herein, the antibody is afucosylated. In any one of the embodiments herein, the afucosylated antibody is stably expressed in an engineered cell, wherein the engineered cell overexpresses a Pseudomonas enzyme GDP-6-deoxy-D-lyxo-4-hexylose reductase (RMD). In any one of the embodiments herein, the active Fc region comprises one or more amino acid substitutions in the Fc region.

In any one of the embodiments herein, the antibody is at least one of: a neutral antibody, a monoclonal antibody, an antagonistic antibody, an agonist antibody, a polyclonal antibody, an IgG1 antibody, an IgG2 antibody, an IgG3 antibody, a bispecific antibody, a human antibody, a humanized antibody, a chimeric antibody, a full length antibody, and an antigen binding fragment.

In any one of the embodiments herein, the antibody has antibody-dependent cell-mediated cytotoxicity (ADCC) activity. In any one of the embodiments herein, the antibody has complement-dependent cytotoxicity (CDC) activity. In any one of the embodiments herein, antibody has antibody-mediated phagocytosis (ADCP) activity. In any one of the embodiments herein, the antibody has receptor-ligand blocking, agonism, or antagonism activity.

In any one of the embodiments herein, the antibody binds the extracellular domain of TREM1. In any one of the embodiments herein, the antibody binds to TREM1 with a K_{D} less than or equal to 0.2 nM. In any one of the embodiments herein, the antibody binds to TREM1 with a K_{D} less than or equal to 0.09 nM, 0.11 nM, or 0.15 nM.

In any one of the embodiments herein, the myeloid cells are stimulatory myeloid cells. In any one of the embodiments herein, the myeloid cells are non-stimulatory myeloid cells. In any one of the embodiments herein, the myeloid cells comprise at least one of dendritic cells, tumor-associated macrophages (TAMs), neutrophils, or monocytes. In any one of the embodiments herein, the myeloid cells are neutrophils. In any one of the embodiments herein, the myeloid cells are tumor-associated macrophages.

In any one of the embodiments herein, the myeloid cells are intratumoral. In any one of the embodiments herein, the myeloid cells are in a population of immune cells comprising stimulatory myeloid cells and non-stimulatory myeloid cells.

In any one of the embodiments herein, the myeloid cells comprise cells that are at least one of (i) CD45⁺, HLA-DR⁻, CD15⁺; (ii) CD45⁺, HLA-DR⁻, CD16⁺, CD56⁻, NKp44⁻, NKp30⁻, NKp46⁻, NKp80⁻; (iii) CD45⁺, HLA-DR⁻, CD14⁺; (iv) CD45⁺, HLA-DR⁺, and CD14⁺; (v) CD45⁺ HLA-DR⁺, CD16⁺, CD56⁻, NKp44⁻, NKp30⁻, NKp46⁻, NKp80⁻; (vi) CD45⁺ HLA-DR⁺CD14⁺, CD16⁺; (vii) CD45⁺, HLA-DR⁻, CD66b⁺; (viii) CD45⁺, HLA-DR⁺, CD14⁺, BDCA3⁻, CD11b⁺, and CD11e⁺; (ix) CD45⁺, CD14⁺, HLA-DR⁺, CD68^{high}, CD20⁻; and (x) CD45⁺, HLA-DR⁺, CD14⁻, CD11c⁺, and BDCA1⁺.

In any one of the embodiments herein, the contacting induces apoptosis, lysis, or growth arrest of the myeloid cells. In any one of the embodiments herein, the contacting occurs in vivo in a subject in need thereof, optionally wherein the subject has cancer.

In any one of the embodiments herein, the cancer is a solid cancer. In any one of the embodiments herein, the cancer is a liquid cancer. In any one of the embodiments herein, the cancer is selected from the group consisting of: melanoma, kidney, hepatobiliary, head-neck squamous carcinoma (HNSC), pancreatic, colon, bladder, glioblastoma, prostate, lung, breast, ovarian, gastric, kidney, bladder, esophageal, renal, melanoma, and mesothelioma. In any one of the embodiments herein, the cancer is colon cancer or breast cancer.

In any one of the embodiments herein, the contacting enhances an immune response in the subject. In any one of the embodiments herein, the enhanced immune response is an adaptive immune response. In any one of the embodiments herein, the enhanced immune response is an innate immune response.

In any one of the embodiments herein, the subject has previously received, is concurrently receiving, or will subsequently receive an immunotherapy. In any one of the embodiments herein, the immunotherapy is at least one of: a checkpoint inhibitor; a checkpoint inhibitor of T cells; anti-PD1; anti-PDL1; anti-CTLA4; adoptive T cell therapy; CAR-T cell therapy; a dendritic cell vaccine; a monocyte vaccine; an antigen binding protein that binds both a T cell and an antigen presenting cell; a BiTE dual antigen binding protein; a toll-like receptor ligand; a cytokine; a cytotoxic therapy; a chemotherapy; a radiotherapy; a small molecule inhibitor; a small molecule agonist; an immunomodulator; and an epigenetic modulator. In any one of the embodiments herein, the immunotherapy is anti-PD1.

In any one of the embodiments herein, the contacting is in vitro or in vivo. In any one of the embodiments herein, the subject is human.

In any one of the embodiments herein, the antibody is conjugated to at least one therapeutic agent selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide, an antisense molecule, a siRNA, a second antibody and a second antibody fragment.

In any one of the embodiments herein, the method does not induce substantial peripheral neutropenia in the subject. In any one of the embodiments herein, the subject's neutrophil levels in the periphery remain substantially the same after contacting with the anti-TREM1 antibody compared to before contacting with the anti-TREM1 antibody.

In another aspect, provided herein are methods of killing non-stimulatory myeloid cells, comprising contacting the non-stimulatory myeloid cells with an anti-Triggering Receptor Expressed on Myeloid Cells 1 (TREM1) antibody, thereby killing the non-stimulatory myeloid cells.

In another aspect, provided herein are methods of disabling non-stimulatory myeloid cells, comprising contacting the non-stimulatory myeloid cells with an anti-TREM1 antibody, thereby disabling the non-stimulatory myeloid cells.

In another aspect, provided herein are methods of increasing the ratio of stimulatory myeloid cells to non-stimulatory myeloid cells in a population of immune cells comprising stimulatory myeloid cells and non-stimulatory myeloid cells, comprising contacting the population of immune cells with an anti-TREM1 antibody, thereby increasing the ratio of stimulatory myeloid cells to non-stimulatory myeloid cells.

In another aspect, provided herein are methods of reducing the number of non-stimulatory myeloid cells in a population of immune cells comprising stimulatory myeloid cells and non-stimulatory myeloid cells, comprising contacting the population of immune cells with an anti-TREM1 antibody, thereby reducing the number of non-stimulatory myeloid cells.

In any one of the embodiments herein, the non-stimulatory myeloid cells are in a population of immune cells comprising stimulatory myeloid cells and non-stimulatory myeloid cells.

In any one of the embodiments herein, the method further comprises removing the non-stimulatory myeloid cells from the population of immune cells.

In any one of the embodiments herein, the non-stimulatory myeloid cells are tumor-associated macrophages.

In any one of the embodiments herein, the non-stimulatory myeloid cells are dendritic cells.

In any one of the embodiments herein, the non-stimulatory myeloid cells are CD45+, HLA-DR+, and CD14+. In any one of the embodiments herein, the non-stimulatory myeloid cells are CD45+, HLA-DR⁺, CD14+, BDCA3−, CD11b+, and CD11c+. In any one of the embodiments herein, the non-stimulatory myeloid cells are CD45+, HLA-DR+, CD14−, CD11c+, and BDCA1+. In any one of the embodiments herein, the non-stimulatory myeloid cells are not CD45+, HLA-DR+, CD14−, CD11c+, and BDCA3+.

In any one of the embodiments herein, the antibody has antibody-dependent cell-mediated cytotoxicity (ADCC) activity. In any one of the embodiments herein, the antibody has complement-dependent cytotoxicity (CDC) activity. In any one of the embodiments herein, the antibody has antibody-mediated phagocytosis activity.

In any one of the embodiments herein, the antibody is a monoclonal antibody. In any one of the embodiments herein, the antibody is a polyclonal antibody. In any one of the embodiments herein, the antibody is an IgG1 antibody. In any one of the embodiments herein, the antibody is an IgG3 antibody. In any one of the embodiments herein, the antibody is not an IgG2 antibody. In any one of the embodiments herein, the antibody is not an IgG4 antibody. In any one of the embodiments herein, the antibody is a bispecific antibody. In any one of the embodiments herein, the antibody is a human antibody. In any one of the embodiments herein, the antibody is a humanized antibody. In any one of the embodiments herein, the antibody is full length. In any one of the embodiments herein, the antibody is a fragment. In any one of the embodiments herein, the antibody is conjugated.

In any one of the embodiments herein, the antibody is conjugated to at least one therapeutic agent selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide, an antisense molecule, a siRNA, a second antibody and a second antibody fragment.

In any one of the embodiments herein, the antibody is selective for TREM1.

In any one of the embodiments herein, the antibody is an antagonistic antibody.

In any one of the embodiments herein, the contacting induces apoptosis of the non-stimulatory myeloid cells. In any one of the embodiments herein, the contacting induces lysis of the non-stimulatory myeloid cells. In any one of the embodiments herein, the contacting induces growth arrest in the non-stimulatory myeloid cells.

In any one of the embodiments herein, the stimulatory myeloid cells comprise cells that are CD45+, HLA-DR+, CD14−, CD11c+, and BDCA3+.

In any one of the embodiments herein, the non-stimulatory myeloid cells are in a cancer tissue. In any one of the embodiments herein, the population of immune cells is in a cancer tissue. In any one of the embodiments herein, the cancer is a solid cancer. In any one of the embodiments herein, the cancer is a liquid cancer.

In any one of the embodiments herein, the cancer is selected from the group consisting of melanoma, kidney, hepatobiliary, head-neck squamous carcinoma (HNSC), pancreatic, colon, bladder, glioblastoma, prostate, lung, and breast.

In any one of the embodiments herein, the contacting is in vitro. In any one of the embodiments herein, the contacting is in vivo. In any one of the embodiments herein, the in vivo is in a human and wherein the contacting is effected by administering the antibody.

In another aspect, provided herein are methods of treating cancer in an individual comprising administering to the individual an effective amount of a composition comprising an anti-TREM1 antibody.

In another aspect, provided herein are methods of enhancing an immune response in an individual comprising administering to the individual an effective amount of a composition comprising an anti-TREM1 antibody. In any one of the embodiments herein, the antibody has antibody-dependent cell-mediated cytotoxicity (ADCC) activity. In any one of the embodiments herein, the antibody has complement-dependent cytotoxicity (CDC) activity. In any one of the embodiments herein, the antibody has antibody-mediated phagocytosis activity.

In any one of the embodiments herein, the antibody is a monoclonal antibody. In any one of the embodiments herein, the antibody is a polyclonal antibody. In any one of the embodiments herein, the antibody is an IgG1 antibody. In any one of the embodiments herein, the antibody is an IgG3 antibody. In any one of the embodiments herein, the antibody is not an IgG2 antibody. In any one of the embodiments herein, the antibody is not an IgG4 antibody. In any one of the embodiments herein, the antibody is a bispecific antibody. In any one of the embodiments herein, the antibody is a human antibody. In any one of the embodiments herein, the antibody is a humanized antibody. In any one of the embodiments herein, the antibody is full length. In any one of the embodiments herein, the antibody is a fragment. In any one of the embodiments herein, the antibody is conjugated.

In any one of the embodiments herein, the antibody is conjugated to at least one therapeutic agent selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide, an antisense molecule, a siRNA, a second antibody and a second antibody fragment.

In any one of the embodiments herein, the antibody is selective for TREM1.

In any one of the embodiments herein, the antibody is an antagonistic antibody.

In any one of the embodiments herein, the administering of the antibody results the killing of non-stimulatory myeloid cells in the individual, disabling non-stimulatory myeloid cells in the individual, or increasing the ratio of stimulatory myeloid cells to non-stimulatory myeloid cells in the individual.

In any one of the embodiments herein, the non-stimulatory cells and stimulatory myeloid cells are in a cancer tissue.

In any one of the embodiments herein, the biological sample is derived from cancer tissue.

In any one of the embodiments herein, the cancer is a solid cancer.

In any one of the embodiments herein, the cancer is a liquid cancer.

In any one of the embodiments herein, the cancer is selected from the group consisting of melanoma, kidney, hepatobiliary, head-neck squamous carcinoma (HNSC), pancreatic, colon, bladder, glioblastoma, prostate, lung, and breast.

In any one of the embodiments herein, the administering of the antibody results in the killing of non-stimulatory myeloid cells in the individual.

In any one of the embodiments herein, the non-stimulatory myeloid cells are CD45+, HLA-DR+, and CD14+. In any one of the embodiments herein, the non-stimulatory myeloid cells are CD45+, HLA-DR⁺, CD14+, BDCA3−, CD11b+, and CD11c+. In any one of the embodiments herein, the non-stimulatory myeloid cells are CD45+, HLA-DR+, CD14−, CD11c+, and BDCA1+. In any one of the embodiments herein, non-stimulatory myeloid cells are not CD45+, HLA-DR+, CD14−, CD11c+, and BDCA3+.

In any one of the embodiments herein, further comprising determining the expression level of TREM1 in a biological sample from the individual. In any one of the embodiments herein, the expression level of TREM1 comprises the mRNA expression level of TREM1. In any one of the embodiments herein, the expression level of TREM1 comprises the protein expression level of TREM1.

In another aspect, provided herein are an antibody which binds a TREM1 protein and is capable of disabling non-stimulatory myeloid cells.

In any one of the embodiments herein, the disabling is by: a) killing of the non-stimulatory myeloid cells; b) magnetic bead depletion of the non-stimulatory myeloid cells; or c) FACS sorting of the non-stimulatory myeloid cells.

In any one of the embodiments herein, the antibody neutralizes a biological activity of TREM1.

In any one of the embodiments herein, the TREM1 is expressed on the surface of non-stimulatory myeloid cells. In any one of the embodiments herein, the non-stimulatory myeloid cells are tumor-associated macrophages. In any one of the embodiments herein, the non-stimulatory myeloid cells are dendritic cells.

In any one of the embodiments herein, the non-stimulatory myeloid cells are CD45+, HLA-DR+, and CD14+. In any one of the embodiments herein, the non-stimulatory myeloid cells are CD45+, HLA-DR⁺, CD14+, BDCA3−, CD11b+, and CD11c+. In any one of the embodiments herein, the non-stimulatory myeloid cells are CD45+, HLA-DR+, CD14−, CD11c+, and BDCA1+. In any one of the embodiments herein, the non-stimulatory myeloid cells are not CD45+, HLA-DR+, CD14−, CD11c+, and BDCA3+.

In any one of the embodiments herein, the antibody has antibody-dependent cell-mediated cytotoxicity (ADCC) activity. In any one of the embodiments herein, the antibody has complement-dependent cytotoxicity (CDC) activity. In any one of the embodiments herein, the antibody has antibody-mediated phagocytosis activity.

In any one of the embodiments herein, the antibody is a monoclonal antibody. In any one of the embodiments herein, the antibody is a polyclonal antibody. In any one of the embodiments herein, the antibody is an IgG1 antibody. In any one of the embodiments herein, the antibody is an IgG3 antibody. In any one of the embodiments herein, the antibody is not an IgG2 antibody. In any one of the embodiments herein, the antibody is not an IgG4 antibody. In any one of the embodiments herein, the antibody is a bispecific antibody. In any one of the embodiments herein, the antibody is a human antibody. In any one of the embodiments herein, the antibody is a humanized antibody. In any one of the embodiments herein, the antibody is full length. In any one of the embodiments herein, the antibody is a fragment. In any one of the embodiments herein, the antibody is conjugated.

In any one of the embodiments herein, the antibody is conjugated to at least one therapeutic agent selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide, an antisense molecule, a siRNA, a second antibody and a second antibody fragment.

In any one of the embodiments herein, the antibody is selective for TREM1.

In any one of the embodiments herein, the antibody is an antagonistic antibody.

In another aspect, provided herein are a pharmaceutical composition comprising any one of the antibodies provided herein and a pharmaceutically acceptable excipient. In one embodiment, the composition is sterile.

In another aspect, provided herein are a kit comprising the any one of the antibodies described herein, further comprising instructions for use. In any one of the embodiments herein, the kit further contains a component selected from a group comprising secondary antibodies, reagents for immunohistochemistry analysis, pharmaceutically acceptable excipient and instruction manual and any combination thereof.

In another aspect, provided herein are an article of manufacture comprising any one of the compositions described herein.

In another aspect, the present invention provides a method of determining the presence or absence of non-stimulatory myeloid cells comprising: contacting a population of immune cells comprising non-stimulatory myeloid cells and stimulatory myeloid cells with an anti-TREM1 antibody; determining the presence of complexes indicating the binding of the antibody to non-stimulatory myeloid cells; and optionally quantifying the number of non-stimulatory myeloid cells in the population.

In another aspect, the present invention provides a method of identifying an individual who may respond to immunotherapy for the treatment of cancer comprising: detecting the expression level of TREM1 in a biological sample from the individual; and determining based on the expression level of TREM1, whether the individual may respond to immunotherapy, wherein an elevated level of TREM1 in the individual relative to that in a healthy individual indicates that the individual may respond immunotherapy.

In any one of the embodiments herein, the immunotherapy comprises treatment with an anti-TREM1 antibody. In any one of the embodiments herein, the expression level of TREM1 comprises the mRNA expression level of TREM1. In any one of the embodiments herein, the expression level of TREM1 comprises the protein expression level of TREM1.

### 5. BRIEF DESCRIPTION OF THE DRAWINGS

FIG. 1 shows the results of recombinant and cell-based specific TREM1 binding, epitope binning, and ADCC/ADCP functional experiments with anti-mTREM1 antibodies.

FIG. 2 shows the anti-tumor activity of PI-9069S treatment as a monotherapy in the MC38 model compared to isotype control and PI-9067s.

FIG. 3 shows the responses of individual MC38 tumor-bearing mice to isotype, PI-9067s and PI-9069s monotherapy treatment.

FIG. 4 shows anti-tumor activity of PI-9069L and anti-PD1 combination treatment in the CT26 model relative to controls.

FIG. 5 shows the responses of individual CT26 tumor-bearing mice to combination treatment with PI-9069L and anti-PD1.

FIG. 6 shows a schematic of the experimental design of intratumoral receptor occupancy and pharmacodynamics studies with PI-9069s or L in four distinct syngeneic tumor models.

FIG. 7 shows that PI-9069s or PI-9069L increases intratumoral TAM and Monocyte depletion scores.

FIG. 8 shows that PI-9069s or PI-9069L demonstrates intratumoral myeloid-specific RO activity in four distinct syngeneic tumor models.

FIG. 9 shows that PI-9069s or PI-9069L demonstrates intratumoral myeloid-specific PD activity in four distinct syngeneic tumor models.

FIG. 10 shows a dose escalation efficacy experiment of PI-9069L in the MC38 model and that efficacy correlates with depletion of intratumoral monocytes.

FIG. 11 shows that PI-9069L significantly reduces monocytes, but not neutrophils, in the blood and bone marrow of naïve mice and may be depleting monocyte progenitors.

FIG. 12 shows the results of cell-based specific TREM1 binding, epitope binning, and ADCC/ADCP functional experiments with anti-hTREM1 antibodies.

FIG. 13 shows that TREM1 is highly expressed on TAMs relative to DC and lymphocytes from primary human tumor samples.

FIG. 14 shows the results of the binning experiments with anti-hTREM1 antibodies.

FIG. 15 shows that PI-8421 binds to hTREM1 but not cTREM1.

FIG. 16 shows that PI-8421 and 0170 have similar ADCC and ADCP activities in a reporter assay system.

FIG. 17 shows that PI-8421 induces ADCC and ADCP activity in a primary macrophage setting. HEK(hTREM1)×PI-8421 is far left. HEK(hTREM1)×isotype is middle left. HEK(Control)×PI-8421 is middle right. HEK(control)×isotype is far right.

FIG. 18 shows the characterization of various mouse monoclonal antibodies (mAbs). FIG. 18A shows flow cytometry-based epitope binning of anti-mouse TREM1 mAbs. (+) indicates cross-blocking, and (−) indicates non-cross-blocking. FIG. 18B shows domain mapping of anti-mouse TREM1 mAb binding. “Yes” indicates binding, “No” indicates no binding. FIG. 18C shows a summary of the biochemical characterization of three mAbs.

FIG. 19 shows anti-mouse TREM1 mAb binding and their specificity to neutrophils in BALB/c splenocyte preparations.

FIG. 20 shows a comparison of the binding and functional properties of wild-type or afucosylated anti-mouse TREM1 mAbs in cell-binding and ADCC/ADCP reporter assays.

FIG. 21 shows a schematic of a mouse syngeneic mouse tumor study design.

FIG. 22 demonstrates intra-tumoral receptor occupancy on neutrophils in the CT26 and MC38 models for PI-9067L (FIGS. 22A and 22B) and PI-4928 (FIGS. 22C and 22D).

FIG. 23 shows in vivo treatment results for PI-9067L. PI-9067L reduces neutrophil and conventional monocyte numbers (FIGS. 23A and 23B), but not T and NK cell numbers (FIG. 23C) in the TME of CT26 tumors. Absolute numbers of indicated immune populations are shown. Each group represents at least 5 mice.

FIG. 24 shows PI-9067L-mediated anti-tumor activity in combination with anti-PD-1 in the CT26 syngeneic mouse tumor model. Afucosylation of PI-9067L improves anti-tumor activity in combination with anti-PD-1 (FIG. 24A). Shown are the average tumor volumes (10 mice/group). FIGS. 24B and 24C show individual tumor volumes for PI-9067L and Afuc-PI-9067L in combination with anti-PD-1, respectively.

FIG. 25 shows Afuc-PI-4928-mediated anti-tumor activity in combination with anti-PD-1 in the CT26 syngeneic mouse tumor model. FIG. 25A shows afucosylation of PI-4928 improves ant-tumor activity in combination with anti-PD-1 relative to control groups. Shown are the average tumor volumes (10 mice/group). FIG. 25B shows individual tumor volumes for each group at the end of the study.

FIG. 26 shows tumor volume over time in C57BL/6 mice implanted with MC38 tumor cells and treated with indicated mAbs. Afuc-PI-9067L does not show enhanced therapeutic efficacy compared to PI-9067L (FIG. 26A). Each curve represents 10 mice per group. Individual tumor volumes are shown for each group at the end of the study (FIG. 26B).

FIG. 27 shows tumor volume over time in C57BL/6 mice implanted with Py8119 cells and treated with Isotype (FIG. 27A), anti-PD-1 (FIG. 27B), Afuc-PI-4928 (FIG. 27C), or Afuc-PI-4928+anti-PD-1 (FIG. 27D). Individual growth curves of each mouse in each group is shown. FIG. 27E shows the endpoint tumor volumes of each group at Day 28.

FIG. 28 shows in vivo plasma levels of PI-9067L and PI-4928 in CT26 (FIG. 28A) and MC38 (FIG. 28B) tumor bearing mice after dosing with the indicated antibody or isotype control. FIG. 28C shows in vivo plasma concentrations of wild type and afucosylated PI-9067L and PI-4928 in CT26 tumor bearing mice.

FIG. 29 shows the average (mean+/−SD of 10 mice per group) body weight measurements of mice treated with PI-9067L, Afuc-PI-9067L, or combination with anti-PD-1 from the indicated time points from studies in the CT26 (FIG. 29A) and MC38 models (FIG. 29B). FIG. 29C shows the average body weight measurements of mice treated with Afuc-PI-4928 alone or in combination with anti-PD-1 from the indicated time points in the Py8119 model.

FIG. 30 shows neutrophil levels in naïve mice (FIG. 30A), CT26 (FIG. 30B) or MC38 (FIG. 30C) tumor-bearing mice treated with two doses of the indicated mAbs. Neutrophil numbers were enumerated by flow cytometry based on Ly6G and CD11b gating. n.s. denotes not significant.

These and other aspects and advantages of the present invention will become apparent from the subsequent detailed description and claims. It is to be understood that one, some, or all of the properties of the various embodiments described herein may be combined to form other embodiments of the present invention.

### 6. DETAILED DESCRIPTION

Provided herein are methods and compositions for an enhancing an immune response and/or for the treatment of cancer in an individual, comprising disabling (e g killing) myeloid cells using an anti-TREM1 antibody. Also provided herein are methods and compositions for increasing the ratio of stimulatory myeloid cells to non-stimulatory myeloid cells using an anti-TREM1 antibody. Also provided herein are methods and compositions for detecting myeloid cells using an anti-TREM1 antibody. Also provided herein are method for determining the presence or absence of non-stimulatory myeloid cells comprising: contacting a population of immune cells comprising non-stimulatory myeloid cells and stimulatory myeloid cells with an anti-TREM1 antibody; detecting a complex or moiety indicating the binding of the antibody to the cell and optionally quantifying the number of non-stimulatory myeloid cells in the population. Also provided herein are methods and compositions for identifying an individual who may respond to a therapy that enhances the immune response, using an anti-TREM1 antibody.

Unless defined otherwise, technical and scientific terms used herein have the same meaning as commonly understood by one of ordinary skill in the art to which this invention belongs.

The practice of the present invention will employ, unless otherwise indicated, conventional techniques of general chemistry, inorganic chemistry, organic chemistry, medicinal chemistry, protein biology, protein chemistry, pharmacology, molecular biology, microbiology, cell biology, biochemistry, and immunology, which are within the skill of the art.

### 6.1. Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s) ±one standard deviation of that value(s).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies. In some embodiments, the antibody comprises an alternative scaffold. In some embodiments, the antibody consists of an alternative scaffold. In some embodiments, the antibody consists essentially of an alternative scaffold. In some embodiments, the antibody comprises an antibody fragment. In some embodiments, the antibody consists of an antibody fragment. In some embodiments, the antibody consists essentially of an antibody fragment. A “TREM1 antibody,” “anti-TREM1 antibody,” or “TREM1-specific antibody” is an antibody, as provided herein, which specifically binds to the antigen TREM1. In some embodiments, the antibody binds the extracellular domain of TREM1. In certain embodiments, a TREM1 antibody provided herein binds to an epitope of TREM1 that is conserved between or among TREM1 proteins from different species.

The term “antigen-binding domain” means the portion of an antibody that is capable of specifically binding to an antigen or epitope. One example of an antigen-binding domain is an antigen-binding domain formed by a V_{H}-V_{L} dimer of an antibody. Another example of an antigen-binding domain is an antigen-binding domain formed by diversification of certain loops from the tenth fibronectin type III domain of an Adnectin. An antigen-binding domain can include CDRs 1, 2, and 3 from a heavy chain in that order; and CDRs 1, 2, and 3 from a light chain in that order.

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The term “Fc region” or “Fc” means the C-terminal region of an immunoglobulin heavy chain that, in naturally occurring antibodies, interacts with Fc receptors and certain proteins of the complement system. The structures of the Fc regions of various immunoglobulins, and the glycosylation sites contained therein, are known in the art. See Schroeder and Cavacini, J. Allergy Clin. Immunol., 2010, 125:S41-52, incorporated by reference in its entirety. The Fc region may be a naturally occurring Fc region, or an Fc region modified as described in the art or elsewhere in this disclosure.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa (κ) and lambda (λ), based on the sequence of its constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme); each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

TABLE 1
Residues in CDRs according to Kabat and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat | H31-H35B | H26-H32 or H34* |
| Numbering) | | |
| H1 (Chothia | H31-H35 | H26-H32 |
| Numbering) | | |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen-binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with β-mercaptoethanol.

“Single-chain Fv” or “sFv” or “scFv” antibody fragments comprise a V_{H} domain and a V_{L} domain in a single polypeptide chain. The V H and V L are generally linked by a peptide linker See Plückthun A. (1994). Any suitable linker may be used. In some embodiments, the linker is a (GGGGS)ₙ (SEQ ID NO: 73). In some embodiments, n=1, 2, 3, 4, 5, or 6. See Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminal of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG4 Fc domain.

The term “single domain antibody” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies.

A “multispecific antibody” is an antibody that comprises two or more different antigen-binding domains that collectively specifically bind two or more different epitopes. The two or more different epitopes may be epitopes on the same antigen (e.g., a single TREM1 molecule expressed by a cell) or on different antigens (e.g., different TREM1 molecules expressed by the same cell, or a TREM1 molecule and a non-TREM1 molecule). In some aspects, a multi-specific antibody binds two different epitopes (i.e., a “bispecific antibody”). In some aspects, a multi-specific antibody binds three different epitopes (i.e., a “trispecific antibody”).

A “monospecific antibody” is an antibody that comprises one or more binding sites that specifically bind to a single epitope. An example of a monospecific antibody is a naturally occurring IgG molecule which, while divalent (i.e., having two antigen-binding domains), recognizes the same epitope at each of the two antigen-binding domains. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

“Affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope). The affinity of a molecule X for its partner Y can be represented by the dissociation equilibrium constant (K D). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. In some aspects, the affinity of a TREM1 antibody for a non-target molecule is less than about 50% of the affinity for TREM1. In some aspects, the affinity of a TREM1 antibody for a non-target molecule is less than about 40% of the affinity for TREM1. In some aspects, the affinity of a TREM1 antibody for a non-target molecule is less than about 30% of the affinity for TREM1. In some aspects, the affinity of a TREM1 antibody for a non-target molecule is less than about 20% of the affinity for TREM1. In some aspects, the affinity of a TREM1 antibody for a non-target molecule is less than about 10% of the affinity for TREM1. In some aspects, the affinity of a TREM1 antibody for a non-target molecule is less than about 1% of the affinity for TREM1. In some aspects, the affinity of a TREM1 antibody for a non-target molecule is less than about 0.1% of the affinity for TREM1.

The term “k_{d}” (sec⁻¹), as used herein, refers to the dissociation rate constant of a particular antibody—antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ, value.

The term “K_{D}” (M), as used herein, refers to the dissociation equilibrium constant of a particular antibody-antigen interaction. K_{D}=k_{d}/kₐ. In some embodiments, the affinity of an antibody is described in terms of the K_{D} for an interaction between such antibody and its antigen. For clarity, as known in the art, a smaller K_{D} value indicates a higher affinity interaction, while a larger K_{D} value indicates a lower affinity interaction.

The term “K_{A}” (M⁻¹), as used herein, refers to the association equilibrium constant of a particular antibody-antigen interaction. K_{A}=kₐ/k_{d}.

An “immunoconjugate” is an antibody conjugated to one or more heterologous molecule(s), such as a therapeutic (cytokine, for example) or diagnostic agent.

“Effector functions” refer to those biological activities mediated by the Fc region of an antibody, which activities may vary depending on the antibody isotype. Examples of antibody effector functions include C1q binding to activate complement dependent cytotoxicity (CDC), Fc receptor binding to activate antibody-dependent cellular cytotoxicity (ADCC), and antibody dependent cellular phagocytosis (ADCP), receptor ligand blocking, agonism, or antagonism.

When used herein in the context of two or more antibodies, the term “competes with” or “cross-competes with” indicates that the two or more antibodies compete for binding to an antigen (e.g., TREM1). In one exemplary assay, TREM1 is coated on a surface and contacted with a first TREM1 antibody, after which a second TREM1 antibody is added. In another exemplary assay, a first TREM1 antibody is coated on a surface and contacted with TREM1, and then a second TREM1 antibody is added. If the presence of the first TREM1 antibody reduces binding of the second TREM1 antibody, in either assay, then the antibodies compete with each other. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 25%, at least 50%, at least 60%, at least 70%, at least 80%, at least 85%, at least 90%, or at least 95%. A skilled artisan can select the concentrations of the antibodies used in the competition assays based on the affinities of the antibodies for TREM1 and the valency of the antibodies. The assays described in this definition are illustrative, and a skilled artisan can utilize any suitable assay to determine if antibodies compete with each other. Suitable assays are described, for example, in Cox et al., “Immunoassay Methods,” in Assay Guidance Manual [Internet], Updated Dec. 24, 2014 (www.ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

The term “epitope” means a portion of an antigen that specifically binds to an antibody. Epitopes frequently consist of surface-accessible amino acid residues and/or sugar side chains and may have specific three dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to TREM1 variants with different point-mutations, or to chimeric TREM1 variants.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations.

For all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as ‘consisting essentially of’ a component, the composition may additionally contain any amount of pharmaceutically acceptable carriers, vehicles, or diluents and other such components which do not substantially affect the condition being treated.

An “effective amount” or “therapeutically effective amount” as used herein refers to an amount of therapeutic compound, such as an anti-TREM1 antibody, administered to an individual, either as a single dose or as part of a series of doses, which is effective to produce or contribute to a desired therapeutic effect, either alone or in combination with another therapeutic modality. Examples of a desired therapeutic effect is enhancing an immune response, slowing or delaying tumor development; stabilization of disease; amelioration of one or more symptoms. An effective amount may be given in one or more dosages.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subject” or “individual” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an antibody provided herein. In some aspects, the disease or condition is a cancer. In some aspects, the disease or condition is a viral infection.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “cytotoxic agent,” as used herein, refers to a substance that inhibits or prevents a cellular function and/or causes cell death or destruction.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “cytostatic agent” refers to a compound or composition which arrests growth of a cell either in vitro or in vivo. In some embodiments, a cytostatic agent is an agent that reduces the percentage of cells in S phase. In some embodiments, a cytostatic agent reduces the percentage of cells in S phase by at least about 20%, at least about 40%, at least about 60%, or at least about 80%.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The terms “cell proliferative disorder” and “proliferative disorder” refer to disorders that are associated with some degree of abnormal cell proliferation. In some embodiments, the cell proliferative disorder is a cancer. In some aspects, the tumor is a solid tumor. In some aspects, the tumor is a hematologic malignancy.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “co-administration”, “co-administer”, and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

For any of the structural and functional characteristics described herein, methods of determining these characteristics are known in the art.

6.2. Myeloid Cells

Provided herein are methods and compositions for disabling, killing, depleting, and/or detecting myeloid cells comprising the use of anti-TREM1 antibody. Also provided herein are methods and compositions for targeting and/or detecting myeloid cells expressing a TREM1 protein. In some embodiments, the myeloid cells are non-stimulatory myeloid cells. In other embodiments, the myeloid cells are stimulatory myeloid cells.

As used herein, non-stimulatory myeloid cells are myeloid cells that are not sufficiently effective at stimulating an immune response (e.g. not as effective at stimulating an anti-tumor response in a tumor microenvironment compared to stimulatory myeloid cells). In some embodiments, non-stimulatory myeloid cells are not as effective at presenting an antigen (e.g. a tumor antigen) to T-cells or not as effective at stimulating tumor specific T-cell responses as compared to a stimulatory myeloid cell. In some embodiments, non-stimulatory myeloid cells can display a decreased ability to uptake, process, and/or present tumor-associated antigens to a T cell as compared to a stimulatory myeloid cell. Non-stimulatory myeloid cells may contain a reduced ability or no ability to re-prime cytotoxic T lymphocytes or in some cases cannot stimulate effective tumor-cell killing Non-stimulatory myeloid cells may display lower expression of gene and cell-surface markers involved in antigen processing, antigen presentation and/or antigen co-stimulation including, without limitation, CD80, CD86, MHCI, and MHCII compared to stimulatory myeloid cells.

Non-stimulatory myeloid cells, when compared to stimulatory myeloid cells, may display the lower expression of genes associated with cross-presentation, co-stimulation, and/or stimulatory cytokines, including, without limitation, any one or more of TAP1, TAP2, PSMB8, PSMB9, TAPBP, PSME2, CD24a, CD274, BTLA, CD40, CD244, ICOSL, ICAM1, TIM3, PDL2, RANK, FLT3, CSF2RB, CSF2RB2, CSF2RA, IL12b, XCR1, CCR7, CCR2, CCL22, CXCL9, and CCL5, and increased expression of anti-inflammatory cytokine IL-10. In some embodiments non-stimulatory myeloid cells are dependent on the transcription factor IRF4 and the cytokines GM-CSF or CSF-1 for differentiation and survival. In some embodiments, non-stimulatory myeloid cells can contribute to tumoral angiogenesis by secreting vascular endothelial growth factor (VEGF) and nitric oxide synthase (NOS) and support tumor growth by secreting epidermal growth factor (EGF).

In some embodiments, the myeloid cells are intratumoral. In some embodiments, the myeloid cells are in a population of immune cells comprising stimulatory myeloid cells and non-stimulatory myeloid cells.

In some embodiments, myeloid cells are tumor-associated macrophages (TAM), neutrophils, monocytes, or subsets of dendritic cells (DC). In some embodiments, the myeloid cell is not a dendritic cell (DC).

In some embodiments, myeloid cells are tumor-associated macrophages (TAMs). TAMs are macrophages present near or within cancerous tumors, and are derived from circulating monocytes or resident tissue macrophages.

In some embodiments the non-stimulatory myeloid cells and the stimulatory myeloid cells are distinguished on the basis of the markers they express, or the markers they selectively express. The expression of a cell surface markers can be described as ‘+’ or ‘positive’. The absence of a cell surface marker can be described as ‘−’ or ‘negative’. The expression of a cell surface marker can be further described as ‘high’ (cells expressing high levels of the makers) or ‘low’ (cells expressing low levels of the markers), which indicates the relative expression of each marker on the cell surface. The level of markers may be determined by various methods known in the art, e g immunostaining and FACS analysis, gene analysis, or gel electrophoresis and Western blotting.

In some embodiments, the myeloid cells are neutrophils.

In some embodiments, myeloid cells are dendritic cells (DCs). In some embodiments, dendritic cells can be distinguished by a spikey or dendritic morphology. In one embodiment, the dendritic cell is at least CD45+, HLA-DR+, CD14−, CD11c+, and BDCA1+(also referred to as DC1 cells). In one embodiment, the dendritic cell is not CD45+, HLA-DR+, CD14−, CD11c+, and BDCA3+(also referred to as DC2 cells). In one embodiment a dendritic cell that is CD45+, HLA-DR+, CD14−, CD11c+, and BDCA3+ is a myeloid cell.

In some embodiments, myeloid cells are tumor associated macrophages. In some embodiments, for example in humans, the tumor associated macrophages are at least CD45+, HLA-DR+, CD14+. In some embodiments the tumor associated macrophages are at least CD45⁺, HLA-DR⁺, CD14⁺, CD11b⁺. In some embodiments the tumor associated macrophages are at least CD45⁺, HLA-DR⁺, CD14⁺, CD11c⁺. In some embodiments the tumor associated macrophages are at least CD45⁺, HLA-DR⁺, CD14⁺, BDCA3⁻. In some embodiments the tumor associated macrophages are at least CD45⁺, HLA-DR⁺, CD14⁺, BDCA3⁻, CD11b⁺. In some embodiments the tumor associated macrophages are at least CD45⁺, HLA-DR⁺, CD14⁺, BDCA3⁻, CD11c⁺. In some embodiments the tumor associated macrophages are at least CD45⁺, HLA-DR⁺, CD14⁺, CD11b⁺, and CD11c⁺. In some embodiments the tumor associated macrophages are at least CD45⁺, HLA-DR⁺, CD14⁺, BDCA3⁻, CD11b⁺, and CD11c⁺.

In some embodiments the methods and compositions of the present invention are useful for targeting TAMs and DCs in other mammals, for example in mice. In one embodiment, for example in mice, the tumor-associated macrophage is at least CD45+, HLA-DR+, CD14+, CD11^{high}, and CD11c^{low} (also referred to as TAM1). In one embodiment, for example in mice, tumor-associated macrophages are at least CD45+, HLA-DR+, CD14+, CD11b^{low}, and CD11c^{high} (also referred to as TAM2). The term “CD11b^{high} macrophages”, as used herein, relates to macrophages expressing high levels of CD11b. The term “CD11b^{low} macrophages,” as used herein, relates to macrophages that express on their surface a level of CD11b that is substantially lower than that of CD11b^{high} macrophages. The term “CD11^{high}”, as used herein, relates to macrophages expressing high levels of CD11c. The term “CD11c^{low} macrophages”, as used herein, relates to macrophages that express on their surface a level of CD11c that is substantially lower than that of CD11c^{high} macrophages.

In some embodiments, the myeloid cells of the invention include one or more of TAM and DC1 cells.

In some embodiments, for example in mice, the myeloid cells of the invention include one or more of TAM1, TAM2, and DC1 cells.

In some embodiments, the myeloid cells are localized within the margins of the tumoral lesions or in the transformed tumor ducts, where they come into contact with cognate T-cells. In one embodiment, the localization of the myeloid cell is modified, so that the cells are no longer localized at the tumor margin or are no longer in contact with T-cells.

In some embodiments, the myeloid cells are in a population of immune cells comprising stimulatory myeloid cells and non-stimulatory myeloid cells. In some embodiments, the non-stimulatory myeloid cells are in a population of immune cells comprising only non-stimulatory myeloid cells. The populations of immune cells of the present invention may be pure, homogenous, heterogeneous, derived from a variety of sources (e.g. diseased tissue, tumor tissue, healthy tissue, cell banks), maintained in primary cell cultures, maintained in immortalized cultures, and/or maintained in ex vivo cultures.

In some embodiments, the non-stimulatory myeloid cells are CD45⁺, HLA-DR⁺, CD14⁻, CD11c⁺, and BDCA1⁺. In some embodiments, the non-stimulatory myeloid cells comprise cells that are CD45⁺, HLA-DR⁺, CD14⁻, CD11c⁺, and BDCA1⁺. In some embodiments, the non-stimulatory myeloid cells consist of cells that are CD45⁺, HLA-DR⁺, CD14⁻, CD11c⁺, and BDCA1⁺. In some embodiments, the non-stimulatory myeloid cells consist essentially of cells that are CD45⁺, HLA-DR⁺, CD14⁻, CD11c⁺, and BDCA1⁺.

In some embodiments, the non-stimulatory myeloid cells are CD45⁺, HLA-DR⁺, CD14⁺, BDCA3⁻. In some embodiments, the non-stimulatory myeloid cells comprise cells that are CD45⁺, HLA-DR⁺, CD14⁺, BDCA3⁻. In some embodiments, the non-stimulatory myeloid cells consist of cells that are CD45⁺, HLA-DR⁺, CD14⁺, BDCA3⁻. In some embodiments, the non-stimulatory myeloid cells consist essentially of cells that are CD45⁺, HLA-DR⁺, CD14⁺, BDCA3⁻.

In some embodiments, the non-stimulatory myeloid cells are CD45⁺, HLA-DR⁺, CD14⁺, CD11b⁺. In some embodiments, the non-stimulatory myeloid cells comprise cells that are CD45⁺, HLA-DR⁺, CD14⁺, CD11b⁺. In some embodiments, the non-stimulatory myeloid cells consist of cells that are CD45⁺, HLA-DR⁺, CD14⁺, CD11b⁺. In some embodiments, the non-stimulatory myeloid cells consist essentially of cells that are CD45⁺, HLA-DR⁺, CD14⁺, CD11b⁺.

In some embodiments, the non-stimulatory myeloid cells are CD45⁺, HLA-DR⁺, CD14⁺, CD11c⁺. In some embodiments, the non-stimulatory myeloid cells comprise cells that are CD45⁺, HLA-DR⁺, CD14⁺, CD11c⁺. In some embodiments, the non-stimulatory myeloid cells consist of cells that are CD45⁺, HLA-DR⁺, CD14⁺, CD11c⁺. In some embodiments, the non-stimulatory myeloid cells consist essentially of cells that are CD45⁺, HLA-DR⁺, CD14⁺, CD11c⁺.

In some embodiments, the non-stimulatory myeloid cells are CD45⁺, HLA-DR⁺, CD14⁺, BDCA3⁻, and CD11c⁺. In some embodiments, the non-stimulatory myeloid cells comprise cells that are CD45⁺, HLA-DR⁺, CD14⁺, BDCA3⁻, and CD11c⁺. In some embodiments, the non-stimulatory myeloid cells consist of cells that are CD45⁺, HLA-DR⁺, CD14⁺, BDCA3⁻, and CD11c⁺. In some embodiments, the non-stimulatory myeloid cells consist essentially of cells that are CD45⁺, HLA-DR⁺, CD14⁺, BDCA3⁻, and CD11c⁺.

In some embodiments, the non-stimulatory myeloid cells are CD45⁺, HLA-DR⁺, CD14⁺, BDCA3⁻, CD11b⁺. In some embodiments, the non-stimulatory myeloid cells comprise cells that are CD45⁺, HLA-DR⁺, CD14⁺, BDCA3⁻, CD11b⁺. In some embodiments, the non-stimulatory myeloid cells consist of cells that are CD45⁺, HLA-DR⁺, CD14⁺, BDCA3⁻, CD11b⁺. In some embodiments, the non-stimulatory myeloid cells consist essentially of cells that are CD45⁺, HLA-DR⁺, CD14⁺, BDCA3⁻, CD11b⁺.

In some embodiments, the non-stimulatory myeloid cells are CD45⁺, HLA-DR⁺, CD14⁺, CD11b⁺, and CD11c⁺. In some embodiments, the non-stimulatory myeloid cells comprise cells that are CD45⁺, HLA-DR⁺, CD14⁺, CD11b⁺, and CD11c⁺. In some embodiments, the non-stimulatory myeloid cells consist of cells that are CD45⁺, HLA-DR⁺, CD14⁺, CD11b⁺, and CD11c⁺. In some embodiments, the non-stimulatory myeloid cells consist essentially of cells that are CD45⁺, HLA-DR⁺, CD14⁺, CD11b⁺, and CD11c⁺.

In some embodiments, the non-stimulatory myeloid cells are CD45⁺, HLA-DR⁺, CD14⁺, BDCA3⁻, CD11b⁺, and CD11c⁺. In some embodiments, the non-stimulatory myeloid cells comprise cells that are CD45⁺, HLA-DR⁺, CD14⁺, BDCA3⁻, CD11b⁺, and CD11c⁺. In some embodiments, the non-stimulatory myeloid cells consist of cells that are CD45⁺, HLA-DR⁺, CD14⁺, BDCA3⁻, CD11b⁺, and CD11c⁺. In some embodiments, the non-stimulatory myeloid cells consist essentially of cells that are CD45⁺, HLA-DR⁺, CD14⁺, BDCA3⁻, CD11b⁺, and CD11c⁺.

In some embodiments, the non-stimulatory myeloid cells are not CD45⁺, HLA-DR⁺, CD14⁻, CD11c⁺, and BDCA3⁺. In some embodiments, the non-stimulatory myeloid cells comprise cells that are not CD45⁺, HLA-DR⁺, CD14⁻, CD11c⁺, and BDCA3⁺.

In some embodiments, for example in mice, the non-stimulatory myeloid cells are CD45⁺, HLA-DR⁺, CD14⁺, CD11b^{high}, and CD11c^{low}. In some embodiments, for example in mice, the non-stimulatory myeloid cells comprise cells that are CD45⁺, HLA-DR⁺, CD14⁺, CD11b^{high}, and CD11c^{low}. In some embodiments, for example in mice, the non-stimulatory myeloid cells consist of cells that are CD45⁺, HLA-DR⁺, CD14⁺, CD11b^{high}, and CD11c^{low}. In some embodiments, for example in mice, the non-stimulatory myeloid cells consist essentially of cells that are CD45⁺, HLA-DR⁺, CD14⁺, CD11b^{high}, and CD11c^{low}.

In some embodiments, for example in mice, the non-stimulatory myeloid cells are CD45⁺, HLA-DR⁺, CD14⁺, CD11b^{low}, and CD11c^{high}. In some embodiments, for example in mice, the non-stimulatory myeloid cells comprise cells that are CD45⁺, HLA-DR⁺, CD14⁺, CD11b^{low}, and CD11c^{high}. In some embodiments, for example in mice, the non-stimulatory myeloid cells consist of cells that are CD45⁺, HLA-DR⁺, CD14⁺, CD11b^{low}, and CD11c^{high}. In some embodiments, for example in mice, the non-stimulatory myeloid cells consist essentially of cells that are CD45⁺, HLA-DR⁺, CD14⁺, CD11b^{low}, and CD11c^{high}.

In some embodiments, the myeloid cells are CD45⁺, HLA-DR⁻, and CD15⁺. In some embodiments, the myeloid cells comprise cells that are CD45⁺, HLA-DR⁻, and CD15⁺. In some embodiments, the myeloid cells consist of cells that are CD45⁺, HLA-DR⁻, and CD15⁺. In some embodiments, the myeloid cells consist essentially of cells that are CD45⁺, HLA-DR⁻, and CD15⁺.

In some embodiments, the myeloid cells are CD45⁺, HLA-DR⁻, CD16⁺, CD56⁻, NKp44⁻, NKp30⁻, NKp46⁻, NKp80⁻. In some embodiments, the myeloid cells comprise cells that are CD45⁺, HLA-DR⁻, CD16⁺, CD56⁻, NKp44⁻, NKp30⁻, NKp46⁻, NKp80⁻. In some embodiments, the myeloid cells consist of cells that are CD45⁺, HLA-DR⁻, CD16⁺, CD56⁻, NKp44⁻, NKp30⁻, NKp46⁻, NKp80⁻. In some embodiments, the myeloid cells consist essentially of cells that are CD45⁺, HLA-DR⁻, CD16⁺, CD56⁻, NKp44⁻, NKp30⁻, NKp46⁻, NKp80⁻.

In some embodiments, the myeloid cells are CD45⁺, HLA-DR⁻, CD14⁺. In some embodiments, the myeloid cells comprise cells that are CD45⁺, HLA-DR⁻, CD14⁺. In some embodiments, the myeloid cells consist of cells that are CD45⁺, HLA-DR⁻, CD14⁺. In some embodiments, the myeloid cells consist essentially of cells that are CD45⁺, HLA-DR⁻, CD14⁺.

In some embodiments, the myeloid cells are CD45⁺, HLA-DR⁺, CD14⁺. In some embodiments, the myeloid cells comprise cells that are CD45⁺, HLA-DR⁺, CD14⁺. In some embodiments, the myeloid cells consist of cells that are CD45⁺, HLA-DR⁺, CD14⁺. In some embodiments, the myeloid cells consist essentially of cells that are CD45⁺, HLA-DR⁺, CD14⁺.

In some embodiments, the myeloid cells are CD45⁺HLA-DR⁺, CD16⁺, CD56⁻, NKp44⁻, NKp30⁻, NKp46⁻, NKp80⁻. In some embodiments, the myeloid cells comprise cells that are CD45⁺HLA-DR⁺, CD16⁺, CD56⁻, NKp44⁻, NKp30⁻, NKp46⁻, NKp80⁻. In some embodiments, the myeloid cells consist of cells that are CD45⁺HLA-DR⁺, CD16⁺, CD56⁻, NKp44⁻, NKp30⁻, NKp46⁻, NKp80⁻. In some embodiments, the myeloid cells consist essentially of cells that are CD45⁺HLA-DR⁺, CD16⁺, CD56⁻, NKp44⁻, NKp30⁻, NKp46⁻, NKp80⁻.

In some embodiments, the myeloid cells are CD45⁺, HLA-DR⁺, CD14⁺, CD16⁺. In some embodiments, the myeloid cells comprise cells that are CD45⁺, HLA-DR⁺, CD14⁺, CD16⁺. In some embodiments, the myeloid cells consist of cells that are CD45⁺, HLA-DR⁺, CD14⁺, CD16⁺. In some embodiments, the myeloid cells consist essentially of cells that are CD45⁺, HLA-DR⁺, CD14⁺, CD16⁺.

In some embodiments, the myeloid cells CD45⁺, HLA-DR⁻, CD66b⁺. In some embodiments, the myeloid cells comprise cells that are CD45⁺, HLA-DR⁻, CD66b⁺. In some embodiments, the myeloid cells consist of cells that are CD45⁺, HLA-DR⁻, CD66b⁺. In some embodiments, the myeloid cells consist essentially of cells that are CD45⁺, HLA-DR⁻, CD66b⁺.

In some embodiments, the myeloid cells are CD45⁺, HLA-DR⁺, CD14⁺, BDCA3⁻, CD11b⁺, CD11c⁺. In some embodiments, the myeloid cells comprise cells that are CD45⁺, HLA-DR⁺, CD14⁺, BDCA3⁻, CD11b⁺, CD11c⁺. In some embodiments, the myeloid cells consist of cells that are CD45⁺, HLA-DR⁺, CD14⁺, BDCA3⁻, CD11b⁺, CD11c⁺. In some embodiments, the myeloid cells consist essentially of cells that are CD45⁺, HLA-DR⁺, CD14⁺, BDCA3⁻, CD11b⁺, CD11c⁺.

In some embodiments, the myeloid cells are CD45⁺, CD14⁺, HLA-DR⁺, CD68^{high}, CD20⁻. In some embodiments, the myeloid cells comprise cells that are CD45⁺, CD14⁺, HLA-DR⁺, CD68^{high}, CD20⁻. In some embodiments, the myeloid cells consist of cells that are CD45⁺, CD14⁺, HLA-DR⁺, CD68^{high}, CD20⁻. In some embodiments, the myeloid cells consist essentially of cells that are CD45⁺, CD14⁺, HLA-DR⁺, CD68^{high}, CD20⁻.

In some embodiments, the myeloid cells are CD45⁺, HLA-DR⁺, CD14⁻, CD11c⁺, BDCA1⁺. In some embodiments, the myeloid cells comprise cells that are CD45⁺, HLA-DR⁺, CD14⁻, CD11c⁺, BDCA1⁺. In some embodiments, the myeloid cells consist of cells that are CD45⁺, HLA-DR⁺, CD14⁻, CD11c⁺, BDCA1⁺. In some embodiments, the myeloid cells consist essentially of cells that are CD45⁺, HLA-DR⁺, CD14⁻, CD11c⁺, BDCA1⁺.

In some embodiments, the myeloid cells are in a cancer tissue. In some embodiments, the population of immune cells is in a cancer tissue. In some embodiments, the non-stimulatory cells and stimulatory myeloid cells are in a cancer tissue. In some embodiments, a biological sample comprises a population of immune cells comprising non-stimulatory myeloid cells and stimulatory myeloid cells.

6.3. Methods of Killing, Disabling, or Depleting Myeloid Cells with a TREM1 Antibody

In some embodiments, the antibody has antibody-dependent cellular cytotoxicity (ADCC) activity. ADCC can occur when antibodies bind to antigens on the surface of pathogenic or tumorigenic target-cells. Effector cells bearing Fc gamma receptors (FcγR or FCGR) on their cell surface, including cytotoxic T-cells, natural killer (NK) cells, macrophages, neutrophils, eosinophils, dendritic cells, or monocytes, recognize and bind the Fc region of antibodies bound to the target-cells. Such binding can trigger the activation of intracellular signaling pathways leading to cell death. In particular embodiments, the antibody's immunoglobulin Fc region subtypes (isotypes) include human IgG1 and IgG3. As used herein, ADCC refers to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Natl. Acad. Sci. (USA) 95:652-656 (1998).

In some embodiments, the antibody has complement-dependent cytotoxicity (CDC) activity. Antibody-induced CDC is mediated through the proteins of the classical complement cascade and is triggered by binding of the complement protein C1q to the antibody. Antibody Fc region binding to C1q can induce activation of the complement cascade. In particular embodiments, the antibody's immunoglobulin Fc region subtypes (isotypes) include human IgG1 and IgG3. As used herein, CDC refers to the ability of a molecule to lyse a target in the presence of complement. The complement activation pathway is initiated by the binding of the first component of the complement system (C1q) to a molecule (e.g. polypeptide (e.g., an antibody)) complexed with a cognate antigen. To assess complement activation, a CDC assay, e.g. as described in Gazzano-Santoro et al., J. Immunol. Methods 202:163 (1996), may be performed.

In some embodiments, the antibody has antibody-dependent cellular phagocytosis (ADCP) activity. ADCP can occur when antibodies bind to antigens on the surface of pathogenic or tumorigenic target-cells. Phagocytic cells bearing Fc receptors on their cell surface, including monocytes and macrophages, recognize and bind the Fc region of antibodies bound to target-cells. Upon binding of the Fc receptor to the antibody-bound target cell, phagocytosis of the target cell can be initiated.

In some embodiments, the antibody has receptor ligand blocking activity. Receptor ligand blocking activity occurs when an antibody binds to a receptor such that the cognate ligand or agonist cannot bind to the receptor and activate it. The antibody may block the receptor by binding to the active site on the receptor, or to an allosteric site on the receptor.

In some embodiments, the antibodies are capable of forming an immune complex. For example, an immune complex can be a tumor cell covered by antibodies.

In one aspect, the present application provides methods of contacting non-stimulatory myeloid cells with an anti-Triggering Receptor Expressed on Myeloid Cells 1 (TREM1) antibody, which results in the disabling of the non-stimulatory myeloid cells.

In some embodiments the non-stimulatory cells are one or more of DC1 cells, and TAM cells.

In some embodiments, the present application provides methods of disabling non-stimulatory myeloid cells, comprising contacting the non-stimulatory myeloid cells with a TREM1 antibody, thereby killing the non-stimulatory myeloid cells. Disabling refers to rendering a cell partially or completely non-functional. In some embodiments, the disabling of the non-stimulatory myeloid cells leads to inducing growth arrest in the cells. In some embodiments, the disabling of the non-stimulatory myeloid cells leads to apoptosis in the cells. In some embodiments, the disabling of the non-stimulatory cells leads to lysis of the cells, as for example by complement dependent cytotoxicity (CDC) or antibody-dependent cell cytotoxicity (ADCC). In some embodiments, the disabling of the non-stimulatory myeloid cells leads to necrosis in the cells. In some embodiments, the disabling of the non-stimulatory myeloid cells leads to inducing growth arrest in the cells. In some embodiments, the disabling of the non-stimulatory myeloid cells leads to inactivating the cells. In some embodiments, the disabling of the non-stimulatory myeloid cells leads to neutralizing the activity of TREM1 in the cells. In some embodiments, the disabling of the non-stimulatory myeloid cells leads to reduction in proliferation of the cells. In some embodiments, the disabling of the non-stimulatory myeloid cells leads to differentiation of the cells. In some embodiments, the disabling of the non-stimulatory myeloid cells leads to a decrease in the cells' ability to act as inhibitory antigen presenting cells or leads to an increase in the cells' ability to act as activating antigen-presenting cells. In some embodiments, the disabling of the non-stimulatory myeloid cells leads to the mislocalization of the cells within tumor tissue or tumor microenvironment (TME). In some embodiments, the disabling of the non-stimulatory myeloid cells leads to an altered spatial organization of the cells within tumor tissue or tumor microenvironment. In some embodiments, the disabling of the non-stimulatory myeloid cells leads to an altered temporal expression of the cells within tumor tissue or TME. In some embodiments, the method further comprises removing the non-stimulatory myeloid cells.

In any and all aspects of disabling non-stimulatory myeloid cells as described herein, any increase or decrease or alteration of an aspect of characteristic(s) or function(s) is as compared to a cell not contacted with an anti-TREM1 antibody.

In another aspect, the present application provides methods of contacting non-stimulatory myeloid cells with an anti-Triggering Receptor Expressed on Myeloid Cells 1 (TREM1) antibody, which results in the modulation of function of the non-stimulatory myeloid cells. The modulation can be any one or more of the following. In some embodiments the non-stimulatory cells are one or more of DC1 cells, TAM1 cells, and TAM2 cells. In some embodiments, the modulation of function leads to the disabling of non-stimulatory myeloid cells. In some embodiments, the modulation of function of the non-stimulatory myeloid cells leads to an increase in the cells' abilities to stimulate both native and activated CD8+ T-cells, for example, by increasing the ability of non-stimulatory cells to cross-present tumor antigen on MHCI molecules to naive CD8+ T-cells. In some embodiments, the modulation increases the T-cell stimulatory function of the non-stimulatory myeloid cells, including, for example, the cells' abilities to trigger T-cell receptor (TCR) signaling, T-cell proliferation, or T-cell cytokine production. In one embodiment, the survival of the non-stimulatory cell is decreased or the proliferation of the non-stimulatory cell is decreased. In one embodiment, the ratio of stimulatory myeloid cells to non-stimulatory myeloid cells is increased.

In any and all aspects of decreasing the function of non-stimulatory myeloid cells as described herein, any increase or decrease or alteration of an aspect of characteristic(s) or function(s) is as compared to a cell not contacted with an anti-TREM1 antibody.

In some embodiments, the present application provides methods of killing (also referred to as inducing cell death) non-stimulatory myeloid cells, comprising contacting the non-stimulatory myeloid cells with an anti-Triggering Receptor Expressed on Myeloid Cells 1 (TREM1) antibody, thereby killing the non-stimulatory myeloid cells. In some embodiments the killing is increased relative to non-stimulatory myeloid cells that have not been contacted with an anti-TREM1 antibody. In some embodiments, the contacting induces apoptosis in the non-stimulatory myeloid cells. In some embodiments, the contacting induces apoptosis in the non-stimulatory myeloid cells. In some embodiments, the non-stimulatory myeloid cells are in a population of immune cells comprising non-stimulatory myeloid cells and stimulatory myeloid cells. In some embodiments, the method further comprises removing the non-stimulatory myeloid cells. In some embodiments, 10%-80% of the cells are killed. In some embodiments, at least 10%, 15%, 20%, 25%, 30%, 35%, 40%, 45%, 50%, 55%, 60%, 65%, 70%, 75%, or 80% of the cells are killed.

In some embodiments, the present application provides methods of increasing the ratio of stimulatory myeloid cells to non-stimulatory myeloid cells in a population of immune cells comprising stimulatory myeloid cells and non-stimulatory myeloid cells, comprising contacting the population of immune cells with an anti-TREM1 antibody. In some embodiments the ratio is increased relative to a population of cells that have not been contacted with an anti-TREM1 antibody. In some embodiments the ratio of DC2 cells to DC1 cells is increased. In some embodiments the ratio of DC2 cells to TAM1 cells is increased. In some embodiments the ratio of DC2 cells to TAM2 cells is increased. In some embodiments the ratio of DC2 cells to TAM1+ TAM2 cells is increased. In some embodiments the ratio of DC2 cells to TAM1+DC1 cells is increased. In some embodiments the ratio of DC2 cells to DC1+TAM2 cells is increased. In some embodiments the ratio of DC2 cells to DC1+ TAM1+ TAM2 cells is increased. In some embodiments, at least the ratio is increased by 10%, 20%, 30%, 40%, 50%, 60%, 70%, 80%, 90%, or 100%.

In some embodiments the ratio of stimulatory myeloid cells to non-stimulatory myeloid cells prior to contacting ranges from 0.001:1-0.1:1. In some embodiments the ratio of stimulatory myeloid cells to non-stimulatory myeloid cells following the contacting ranges from 0.1:1-100:1.

In some embodiments, the non-stimulatory myeloid cells are reduced in number. In some embodiments the stimulatory myeloid cells are DC2 cells. In some embodiments, the non-stimulatory myeloid cells are killed, for example by necrosis, or apoptosis. In some embodiments, the non-stimulatory myeloid cells are induced to undergo growth arrest. In some embodiments the non-stimulatory myeloid cells no longer proliferate. In some embodiments the spatial localization of the non-stimulatory myeloid cells is altered, and the ratio is increased in a particular region of the TME. In some embodiments the temporal expression of the non-stimulatory myeloid cells is altered, and the ratio is increased during a particular time during the development of the tumor.

In some embodiments, the contacting is in vitro. In some embodiments, the contacting is in vivo. In some particular embodiments, the contacting is in vivo in a human. In some embodiments, the contacting is effected by administering an anti-TREM1 antibody. In some embodiments, the individual receiving the antibody (such as a human) has cancer.

In another aspect, the invention provides methods of treating an immune-related condition (e.g., cancer) in an individual comprising administering to the individual an effective amount of a composition comprising an anti-TREM1 antibody. In another aspect, the invention provides methods of enhancing an immune response in an individual comprising administering to the individual an effective amount of a composition comprising an anti-TREM1 antibody. In some embodiments these methods are further provided in combination with other co-therapies such as a PDL blockade therapy, a CTLA4 blockade therapy, generalized checkpoint blockade therapy in which inhibitory molecules on T cells are blocked, adoptive T-cell therapy, CAR T-cell therapy, dendritic cell or other cellular therapies, as well as conventional chemotherapies.

In some embodiments, the method further comprises determining the expression level of TREM1 in a biological sample from the individual. In some embodiments the biological sample includes, but is not limited to a body fluid, a tissue sample, an organ sample, urine, feces, blood, saliva, CSF and any combination thereof. In some embodiments the biological sample is derived from a tumor tissue. In some embodiments, the expression level of TREM1 comprises the mRNA expression level of TREM1. In some embodiments, the expression level of TREM1 comprises the protein expression level of TREM1. In some embodiments the expression level of TREM1 is detected in the sample using a method selected from the group consisting of FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometery, HPLC, qPCR, RT-qPCR, multiplex qPCR or RT-qPCR, RNA-seq, microarray analysis, SAGE, MassARRAY technique, and FISH, and combinations thereof.

In another aspect, the present application provides methods for determining the presence or absence of non-stimulatory myeloid cells in general, or for determining the presence or absence of particular non-stimulatory myeloid cells (for example DC1 cells, TAM 1 cells, and/or TAM2 cells) comprising: contacting a population of cells comprising non-stimulatory myeloid cells with an anti-TREM1 antibody; and quantifying the number non-stimulatory myeloid cells. In another aspect, the present application provides methods for determining the presence or absence of non-stimulatory myeloid cells comprising: contacting a population of immune cells comprising non-stimulatory myeloid cells and stimulatory myeloid cells with an anti-TREM1 antibody; detecting a complex or moiety indicating the binding of the antibody to the cell and optionally quantifying the number of non-stimulatory myeloid cells in the population. In another aspect, methods of determining the relative ratio of non-stimulatory myeloid cells to stimulatory myeloid cells are provided, comprising: contacting a population of immune cells comprising non-stimulatory myeloid cells and stimulatory myeloid cells with an anti-TREM1 antibody; quantifying the number of stimulatory myeloid cells and non-stimulatory myeloid cells; and determining the relative ratio of non-stimulatory myeloid cells to stimulatory myeloid cells.

In embodiments described herein for detection and/or quantification, the anti-TREM1 antibody binds to the TREM1 protein, but does not necessarily have to effect a biological response, such as ADCC, although it may have an effect on a biological response.

In another aspect, the present invention provides methods for identifying an individual who may respond to immunotherapy (e.g. with an anti-TREM1 antibody) for the treatment of an immune-related condition (e.g. cancer) comprising: detecting the expression level of TREM1 in a biological sample from the individual; and determining based on the expression level of TREM1, whether the individual may respond immunotherapy, wherein an elevated level of TREM1 in the individual relative to that in a healthy individual indicates that the individual may respond to immunotherapy. In some embodiments, these methods may also be used for diagnosing an immune-related condition (e.g. cancer) in the individual and are based the expression level of TREM1, wherein an elevated level of TREM1 in the individual relative to that in a healthy individual indicates that the individual suffers from cancer. In some embodiments, the expression level of TREM1 comprises the mRNA expression level of TREM1. In other embodiments, the expression level of TREM1 comprises the protein expression level of TREM1. In some embodiments the expression level of TREM1 is detected in the sample using a method selected from the group consisting of FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometery, HPLC, qPCR, RT-qPCR, multiplex qPCR or RT-qPCR, RNA-seq, microarray analysis, SAGE, MassARRAY technique, and FISH, and combinations thereof. In these embodiments, the anti-TREM1 antibody binds to the TREM1 protein, but does not necessarily have to effect a biological response, such as ADCC. In some embodiments the biological sample is derived from a tumor tissue. In some embodiments the biological sample includes, but is not limited to a body fluid, a tissue sample, an organ sample, urine, feces, blood, saliva, CSF and any combination thereof.

In some embodiments the disabling of non-stimulatory myeloid cells is in vitro and is achieved: a) by killing of the non-stimulatory myeloid cells; b) magnetic bead depletion of the non-stimulatory myeloid cells; or c) Fluorescence-activated cell sorting (FACS) sorting of the non-stimulatory myeloid cells.

In certain embodiments, an antibody provided herein has a dissociation constant (Kd) of range of 0.0001 nM to 1 μM. For example, Kd of the antibody may be about 1 μM, about 100 nM, about 50 nM, about 10 nM, about 1 nM, about 500 pM, about 100 pM, or about 50 pM to any of about 2 pM, about 5 pM, about 10 pM, about 15 pM, about 20 pM, or about 40 pM.

6.4. TREM1 Antibody Compositions

The present application provides antibodies and compositions comprising an antibody which binds a TREM1 protein including antibodies that disable non-stimulatory myeloid cells.

The term “antibody” herein is used in the broadest sense and specifically covers monoclonal antibodies, polyclonal antibodies, multi-specific antibodies (e.g. bispecific antibodies) formed from at least two intact antibodies, antigen-binding antibody fragments, and other antibody fragments so long as they exhibit a desired biological activity. The term “immunoglobulin” (Ig) is used interchangeable with antibody herein. Reference to the term “antibody” herein includes and refers to antibody fragments as well as the antibody forms referred to above. Description and discussion of various antibody characteristics herein uses the term “antibody” and refers to all forms of antibody described. In some embodiments the invention also provides complexes of such antibodies bound to TREM1 and/or a cell expressing TREM1, such as a non-stimulatory myeloid cell as described herein.

Provided herein are antibodies that specifically bind to TREM1. In some aspects, TREM1 is human TREM1. In some embodiments, the antibodies provided herein specifically bind to the extracellular domain of TREM1. The TREM1 may be expressed on the surface of any suitable target cell. In some embodiments, the target cell is a professional antigen presenting cell. In some embodiments, the target cell is a macrophage. An antibody can be pan-specific for human TREM1 isotypes. An antibody can be specific for a human TREM1 isotype.

In certain embodiments an antibody is a wild type anti-TREM1 antibody. In certain embodiments an antibody is an afucosylated anti-TREM1 antibody. In some embodiments, an antibody is a mouse anti-TREM1 antibody. In some embodiments, an antibody is a human anti-TREM1 antibody. In some embodiments, an antibody is a humanized anti-TREM1 antibody.

In some embodiments the antibodies are monoclonal antibodies. In some embodiments the antibodies are polyclonal antibodies. In some embodiments the antibodies are produced by hybridomas. In other embodiments, the antibodies are produced by recombinant cells engineered to express the desired variable and constant domains. In some embodiments the antibodies may be single chain antibodies or other antibody derivatives retaining the antigen specificity and the lower hinge region or a variant thereof.

In some embodiments the antibodies may be polyfunctional antibodies, recombinant antibodies, human antibodies, humanized antibodies, fragments or variants thereof. In particular embodiments, the antibody fragment or a derivative thereof is selected from a Fab fragment, a Fab′2 fragment, a CDR and ScFv.

In some embodiments, the antibodies are specific for surface antigens, such as TREM1. In some embodiments, the therapeutic antibodies are specific for tumor antigens (e.g., molecules specifically expressed by tumor cells). In particular embodiments, the therapeutic antibodies may have human or non-human primate IgG1, IgG2, or IgG3 Fc portions.

In some embodiments, the antibody is bound to, or conjugated to an effector molecule. In particular embodiments, an antibody is conjugated to at least one therapeutic agent selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide, an antisense molecule, a siRNA, a second antibody and a second antibody fragment.

In some embodiments, the antibody is an agonistic antibody. An agonistic antibody can induce (e.g., increase) one or more activities or functions of TREM1 after the antibody binds the TREM1 receptor expressed on the cell. The agonistic antibody may bind to and activate TREM1, causing changes in proliferation of the cell or modifying antigen presentation capabilities. The agonistic antibody may bind to and activate TREM1, triggering intracellular signaling pathways that lead to modified cell growth or apoptosis.

In some embodiments, the antibody is an antagonistic antibody. An antagonistic antibody can block (e.g. decrease) one or more activities or functions of TREM1 after the antibody binds the TREM1 receptor expressed on the cell. For example, the antagonist antibody may bind to and block ligand binding to TREM1, preventing differentiation and proliferation of the cell or modifying antigen presentation capabilities. The antagonist antibody may bind to and prevent activation of TREM1 by its ligand, modifying intracellular signaling pathways that contribute to cell growth and survival.

In some embodiments the antibody is a depleting antibody. A depleting antibody is one that would kill a myeloid cell upon contact through the antibody's interaction with other immune cells of molecules. For example, antibodies, when bound to cells bearing TREM1, could engage complement proteins and induce complement-dependent cell lysis. Antibodies, when bound to cells bearing TREM1, could also triggering neighboring cells bearing Fc receptors to kill them by antibody-dependent cellular cytotoxicity (ADCC).

In some embodiments, the antibody is a neutralizing antibody, and the antibody neutralizes one or more biological activities of TREM1. In some embodiments, TREM1 is expressed on the surface of myeloid cells and the antibody recognizes the extracellular domain of TREM1.

In some embodiments the antibody is selective for TREM1 (preferentially binds to TREM1). In certain embodiments, an antibody that selectively binds to TREM1 has a dissociation constant (Kd) of range of 0.0001 nM to 1 μM. In certain embodiments, an antibody specifically binds to an epitope on a TREM1 protein that is conserved among the protein from different species. In another embodiment, selective binding includes, but does not require, exclusive binding.

In one embodiment an anti-TREM1 antibody bound to its target is responsible for causing the in vivo depletion of non-stimulatory myeloid cells to which it is bound. In some embodiments, effector proteins induced by clustered antibodies can trigger a variety of responses, including release of inflammatory cytokines, regulation of antigen production, endocytosis, or cell killing. In one embodiment the antibody is capable of recruiting and activating complement or mediating antibody-dependent cellular cytotoxicity (ADCC) in vivo, or mediating phagocytosis by binding Fc receptors in vivo. The antibody may also deplete non-stimulatory myeloid cells by inducing apoptosis or necrosis of the non-stimulatory myeloid cell upon binding.

In some embodiments, the antibody may induce apoptosis, lysis, or growth arrest of the myeloid cells.

In some embodiments, the Fc region binds an Fcγ Receptor on an immune cell. In some embodiments, the Fcγ Receptor is FcγRI, FcγRIIa, FcγRIIb, FcγRIIc, FcγRIIIa, or FcγRIIIb. In some embodiments the immune cell is a professional antigen presenting cell, a macrophage, a monocyte, a natural killer cell, a neutrophil, a dendritic cell, or a B cell.

In some embodiments, an antibody binds TREM1 with a K_{D} less than or equal to 0.05-0.2 nM, as measured by Biacore assay. In some embodiments, an antibody binds TREM1 with a K D less than or equal to 0.01 nM, 0.02 nM, 0.03 nM, 0.04 nM, 0.05 nM, 0.06 nM, 0.07 nM, 0.08 nM, 0.09 nM, 0.10 nM, 0.11 nM, 0.12 nM, 0.13 nM, 0.14 nM, 0.15 nM, 0.16 nM, 0.17 nM, 0.18 nM, 0.19 nM, or 0.2 nM. In some embodiments, an antibody binds TREM1 with a K D between 0.01-0.05 nM, 0.05-0.09 nM, 0.08-0.12 nM, 0.11-0.16 nM, or 0.15-0.2 nM. In some embodiments, an antibody binds TREM1 with a K_{D} less than or equal to 0.09 nM, 0.11 nM, or 0.15 nM.

In some embodiments, the antibodies provided herein comprise an active Fc region.

In some aspects, an antibody provided herein comprises an IgG1 domain with reduced fucose content at position Asn 297 compared to a naturally occurring IgG1 domain. Such Fc domains are known to have improved ADCC. See Shields et al., J. Biol. Chem., 2002, 277:26733-26740, incorporated by reference in its entirety. In some aspects, such antibodies do not comprise any fucose at position Asn 297. The amount of fucose may be determined using any suitable method, for example as described in WO 2008/077546, incorporated by reference in its entirety.

Examples of cell lines capable of producing defucosylated antibody include CHO-DG44 with stable overexpression of the bacterial oxidoreductase GDP-6-deoxy-D-lyxo-4-hexylose reductase (RMD) (see Henning von Horsten et al., Glycobiol 2010, 20:1607-1618) or Lec13 CHO cells, which are deficient in protein fucosylation (see Ripka et al., Arch. Biochem. Biophys., 1986, 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al., Biotech. Bioeng., 2004, 87: 614-622; Kanda et al., Biotechnol. Bioeng., 2006, 94:680-688; and WO 2003/085107; each of which is incorporated by reference in its entirety).

In certain embodiments, an antibody provided herein comprises an Fc region with one or more amino acid substitutions which improve ADCC, such as a substitution at one or more of positions 298, 333, and 334 of the Fc region. In some embodiments, an antibody provided herein comprises an Fc region with one or more amino acid substitutions at positions 239, 332, and 330, as described in Lazar et al., Proc. Nall. Acad. Sci. USA, 2006,103:4005-4010, incorporated by reference in its entirety.

In some embodiments, an antibody provided herein comprises one or more alterations that improves or diminishes C1q binding and/or CDC. See U.S. Pat. No. 6,194,551; WO 99/51642; and Idusogie et al., J. Immunol., 2000, 164:4178-4184; each of which is incorporated by reference in its entirety.

In some embodiments, the antibody is an IgG1 antibody. In some embodiments, the antibody is an IgG3 antibody. In some embodiments, the antibody is an IgG2 antibody. In some embodiments, the antibody is an IgG4 antibody.

In some embodiments, the antibodies provided herein comprise a light chain. In some aspects, the light chain is a kappa light chain. In some aspects, the light chain is a lambda light chain.

In some embodiments, the antibodies provided herein comprise a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

### Methods of Making Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3rd ed. (1986) Academic Press, San Diego, CA, incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, CA), and SP-2 or X63-Ag8-653 cells (available from the American Type Culture Collection, Rockville, MD). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

### Methods of Making Chimeric Antibodies

Illustrative methods of making chimeric antibodies are described, for example, in U.S. Pat. No. 4,816,567; and Morrison et al., Proc. Natl. Acad. Sci. USA, 1984, 81:6851-6855; each of which is incorporated by reference in its entirety. In some embodiments, a chimeric antibody is made by using recombinant techniques to combine a non-human variable region (e.g., a variable region derived from a mouse, rat, hamster, rabbit, or non-human primate, such as a monkey) with a human constant region.

Methods of making Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety).

### Methods of Making Antibody Fragments

The antibody fragments provided herein may be made by any suitable method, including the illustrative methods described herein or those known in the art. Suitable methods include recombinant techniques and proteolytic digestion of whole antibodies. Illustrative methods of making antibody fragments are described, for example, in Hudson et al., Nat. Med., 2003, 9:129-134, incorporated by reference in its entirety. Methods of making scFv antibodies are described, for example, in Plückthun, in The Pharmacology of Monoclonal Antibodies, vol. 113, Rosenburg and Moore eds., Springer-Verlag, New York, pp. 269-315 (1994); WO 93/16185; and U.S. Pat. Nos. 5,571,894 and 5,587,458; each of which is incorporated by reference in its entirety.

### Methods of Making Multispecific Antibodies

The multispecific antibodies provided herein may be made by any suitable method, including the illustrative methods described herein or those known in the art. Methods of making common light chain antibodies are described in Merchant et al., Nature Biotechnol., 1998, 16:677-681, incorporated by reference in its entirety. Methods of making tetravalent bispecific antibodies are described in Coloma and Morrison, Nature Biotechnol., 1997, 15:159-163, incorporated by reference in its entirety. Methods of making hybrid immunoglobulins are described in Milstein and Cuello, Nature, 1983, 305:537-540; and Staerz and Bevan, Proc. Natl. Acad. Sci. USA, 1986, 83:1453-1457; each of which is incorporated by reference in its entirety. Methods of making immunoglobulins with knobs-into-holes modification are described in U.S. Pat. No. 5,731,168, incorporated by reference in its entirety. Methods of making immunoglobulins with electrostatic modifications are provided in WO 2009/089004, incorporated by reference in its entirety. Methods of making bispecific single chain antibodies are described in Traunecker et al., EMBO J., 1991, 10:3655-3659; and Gruber et al., J. Immunol., 1994, 152:5368-5374; each of which is incorporated by reference in its entirety. Methods of making single-chain antibodies, whose linker length may be varied, are described in U.S. Pat. Nos. 4,946,778 and 5,132,405, each of which is incorporated by reference in its entirety. Methods of making diabodies are described in Hollinger et al., Proc. Natl. Acad. Sci. USA, 1993, 90:6444-6448, incorporated by reference in its entirety. Methods of making triabodies and tetrabodies are described in Todorovska et al., J. Immunol. Methods, 2001, 248:47-66, incorporated by reference in its entirety. Methods of making trispecific F(ab′)3 derivatives are described in Tutt et al. J. Immunol., 1991, 147:60-69, incorporated by reference in its entirety. Methods of making cross-linked antibodies are described in U.S. Pat. No. 4,676,980; Brennan et al., Science, 1985, 229:81-83; Staerz, et al. Nature, 1985, 314:628-631; and EP 0453082; each of which is incorporated by reference in its entirety. Methods of making antigen-binding domains assembled by leucine zippers are described in Kostelny et al., J. Immunol., 1992, 148:1547-1553, incorporated by reference in its entirety. Methods of making antibodies via the DNL approach are described in U.S. Pat. Nos. 7,521,056; 7,550,143; 7,534,866; and 7,527,787; each of which is incorporated by reference in its entirety. Methods of making hybrids of antibody and non-antibody molecules are described in WO 93/08829, incorporated by reference in its entirety, for examples of such antibodies. Methods of making DAF antibodies are described in U.S. Pat. Pub. No. 2008/0069820, incorporated by reference in its entirety. Methods of making antibodies via reduction and oxidation are described in Carlring et al., PLoS One, 2011, 6:e22533, incorporated by reference in its entirety. Methods of making DVD-IgsTM are described in U.S. Pat. No. 7,612,181, incorporated by reference in its entirety. Methods of making DARTsTM are described in Moore et al., Blood, 2011, 117:454-451, incorporated by reference in its entirety. Methods of making DuoBodies® are described in Labrijn et al., Proc. Natl. Acad. Sci. USA, 2013, 110:5145-5150; Gramer et al., mAbs, 2013, 5:962-972; and Labrijn et al., Nature Protocols, 2014, 9:2450-2463; each of which is incorporated by reference in its entirety. Methods of making antibodies comprising scFvs fused to the C-terminus of the CH3 from an IgG are described in Coloma and Morrison, Nature Biotechnol., 1997, 15:159-163, incorporated by reference in its entirety. Methods of making antibodies in which a Fab molecule is attached to the constant region of an immunoglobulin are described in Miler et al., J. Immunol., 2003, 170:4854-4861, incorporated by reference in its entirety. Methods of making CovX-Bodies are described in Doppalapudi et al., Proc. Natl. Acad. Sci. USA, 2010, 107:22611-22616, incorporated by reference in its entirety. Methods of making Fcab antibodies are described in Wozniak-Knopp et al., Protein Eng. Des. Sel., 2010, 23:289-297, incorporated by reference in its entirety. Methods of making TandAb® antibodies are described in Kipriyanov et al., J. Mol. Biol., 1999, 293:41-56 and Zhukovsky et al., Blood, 2013, 122:5116, each of which is incorporated by reference in its entirety. Methods of making tandem Fabs are described in WO 2015/103072, incorporated by reference in its entirety. Methods of making ZybodiesTM are described in LaFleur et al., mAbs, 2013, 5:208-218, incorporated by reference in its entirety.

### Methods of Making Variants

Any suitable method can be used to introduce variability into a polynucleotide sequence(s) encoding an antibody, including error-prone PCR, chain shuffling, and oligonucleotide-directed mutagenesis such as trinucleotide-directed mutagenesis (TRIM). In some aspects, several CDR residues (e.g., 4-6 residues at a time) are randomized. CDR residues involved in antigen binding may be specifically identified, for example, using alanine scanning mutagenesis or modeling. CDR-H3 and CDR-L3 in particular are often targeted for mutation.

The introduction of diversity into the variable regions and/or CDRs can be used to produce a secondary library. The secondary library is then screened to identify antibody variants with improved affinity. Affinity maturation by constructing and reselecting from secondary libraries has been described, for example, in Hoogenboom et al., Methods in Molecular Biology, 2001, 178:1-37, incorporated by reference in its entirety.

6.5. Individuals

In some embodiments, the methods provided herein are useful for the treatment of an immune-related condition in an individual. In one embodiment, the individual is a human.

In some embodiments, the methods provided herein (such as methods of enhancing an immune response or effecting the disabling of non-stimulatory myeloid cells) are useful for the treatment of cancer and as such an individual receiving the anti-TREM1 antibody has cancer. In some embodiments, the cancer is a solid cancer. In some embodiments, the cancer is a liquid cancer. In some embodiments, the cancer is immunoevasive. In some embodiments, the cancer is immunoresponsive. In particular embodiments, the cancer is selected from the group consisting of melanoma, kidney, hepatobiliary, head-neck squamous carcinoma (HNSC), pancreatic, colon, bladder, prostate, lung, glioblastoma, and breast.

In some embodiments the immune-related condition is an immune-related condition associated with the expression of TREM1 on non-stimulatory myeloid cells. In some embodiments the immune-related condition is an immune-related condition associated with the overexpression of TREM1 on non-stimulatory myeloid cells, as compared to stimulatory myeloid-cells. In some embodiments the overexpression of the TREM1 mRNA or the TREM1 protein is about at least 2 fold, 5 fold, 10 fold, 25 fold, 50 fold, or 100 fold higher as compared to stimulatory myeloid cells.

6.6. Methods of Administration

In some embodiments, the anti-TREM1 antibody is administered intravenously, intramuscularly, subcutaneously, topically, orally, transdermally, intraperitoneally, intraorbitally, by implantation, by inhalation, intrathecally, intraventricularly, or intranasally. An effective amount of the anti-TREM1 antibody may be administered for the treatment of cancer. The appropriate dosage of the anti-TREM1 antibody may be determined based on the type of cancer to be treated, the type of the anti-TREM1 antibody, the severity and course of the cancer, the clinical condition of the individual, the individual's clinical history and response to the treatment, and the discretion of the attending physician.

In some embodiments, for in vivo administration of the anti-TREM1 antibodies described herein, normal dosage amounts may vary from about 10 ng/kg up to about 100 mg/kg of an individual's body weight or more per day, preferably about 1 mg/kg/day to 10 mg/kg/day, depending upon the route of administration. For repeated administrations over several days or longer, depending on the severity of the disease or disorder to be treated, the treatment is sustained until a desired suppression of symptoms is achieved. An exemplary dosing regimen comprises administering an initial dose of an anti-TREM1 antibody of about 2 mg/kg, followed by a weekly maintenance dose of about 1 mg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, and about 2/mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the anti-TREM1 antibody administered, can vary over time independently of the dose used.

6.7. Therapeutic Applications

For therapeutic applications, antibodies are administered to a mammal, generally a human, in a pharmaceutically acceptable dosage form such as those known in the art and those discussed above. For example, antibodies may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The antibodies also are suitably administered by peritumoral, intralesional, or perilesional routes, to exert local as well as systemic therapeutic effects. The intraperitoneal route may be particularly useful, for example, in the treatment of ovarian tumors.

In some embodiments, provided herein is a method of treating a disease or condition in a subject in need thereof by administering an effective amount of an antibody provided herein to the subject. In some aspects, the disease or condition is a cancer.

In some embodiments, provided herein is a method of treating a disease or condition in a subject in need thereof by administering an effective amount of an antibody provided herein to the subject, wherein the disease or condition is a cancer, and the cancer is selected from a solid tumor and a hematological tumor.

In some embodiments, provided herein is a method of increasing phagocytosis in a subject in need thereof, comprising administering to the subject an effective amount of an antibody or a pharmaceutical composition disclosed herein.

In some embodiments, provided herein is a method of modulating an immune response in a subject in need thereof, comprising administering to the subject an effective amount of an antibody or a pharmaceutical composition disclosed herein. In some embodiments, the immune response is enhanced. In some embodiments, the enhanced immune response in an adaptive immune response. In some embodiments, the enhanced immune response is an innate immune response.

Any suitable cancer may be treated with the antibodies provided herein.

Carcinomas are malignancies that originate in the epithelial tissues. Epithelial cells cover the external surface of the body, line the internal cavities, and form the lining of glandular tissues. Examples of carcinomas include, but are not limited to: adenocarcinoma (cancer that begins in glandular (secretory) cells), e.g., cancers of the breast, pancreas, lung, prostate, and colon can be adenocarcinomas; adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, skin, etc.

Soft tissue tumors are a highly diverse group of rare tumors that are derived from connective tissue. Examples of soft tissue tumors include, but are not limited to: alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; and pleomorphic adenoma of soft tissue; and neoplasias derived from fibroblasts, myofibroblasts, histiocytes, vascular cells/endothelial cells and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to: askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; undifferentiated pleomorphic sarcoma, and the like).

A teratomas is a type of germ cell tumor that may contain several different types of tissue (e.g., can include tissues derived from any and/or all of the three germ layers: endoderm, mesoderm, and ectoderm), including for example, hair, muscle, and bone. Teratomas occur most often in the ovaries in women, the testicles in men, and the tailbone in children.

Melanoma is a form of cancer that begins in melanocytes (cells that make the pigment melanin). It may begin in a mole (skin melanoma), but can also begin in other pigmented tissues, such as in the eye or in the intestines.

Leukemias are cancers that start in blood-forming tissue, such as the bone marrow, and causes large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is effected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to: Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One kind is Hodgkin lymphoma (HL), which is marked by the presence of a type of cell called the Reed-Sternberg cell. There are currently 6 recognized types of HL. Examples of Hodgkin lymphomas include: nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to: AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to: gliomas (e.g., glioblastomas, astrocytomas, oligodendrogliomas, ependymomas, and the like), meningiomas, pituitary adenomas, vestibular schwannomas, primitive neuroectodermal tumors (medulloblastomas), etc.

6.8. Combination Therapies

In some embodiments, an antibody provided herein is administered with at least one additional therapeutic agent. Any suitable additional therapeutic agent may be administered with an antibody provided herein. In some aspects, the additional therapeutic agent is selected from radiation, a cytotoxic agent, a chemotherapeutic agent, a cytostatic agent, an anti-hormonal agent, an EGFR inhibitor, an immunostimulatory agent, an anti-angiogenic agent, and combinations thereof.

In some embodiments, the additional therapeutic agent comprises an immunostimulatory agent. In some embodiments, the additional therapeutic agent is an antibody. In some embodiments, the additional therapeutic agent is an antibody that binds a protein or proteins on a tumor cell surface.

For the treatment of cancer, the anti-TREM1 antibody may be combined with one or more antibodies specific for a tumor antigen. Of these, tumor-associated antigens (TAAs) are relatively restricted to tumor cells, whereas tumor-specific antigens (TSAs) are unique to tumor cells. TSAs and TAAs typically are portions of intracellular molecules expressed on the cell surface as part of the major histocompatibility complex.

Tissue specific differentiation antigens are molecules present on tumor cells and their normal cell counterparts. Tumor-associated antigens known to be recognized by therapeutic mAbs fall into several different categories. Hematopoietic differentiation antigens are glycoproteins that are usually associated with cluster of differentiation (CD) groupings and include CD20, CD30, CD33 and CD52. Cell surface differentiation antigens are a diverse group of glycoproteins and carbohydrates that are found on the surface of both normal and tumor cells. Antigens that are involved in growth and differentiation signaling are often growth factors and growth factor receptors. Growth factors that are targets for antibodies in cancer patients include CEA, epidermal growth factor receptor (EGFR; also known as ERBB1), ERBB2 (also known as HER2), ERBB3, MET (also known as HGFR), insulin-like growth factor 1 receptor (IGF1R), ephrin receptor A3 (EPHA3), tumor necrosis factor (TNF)-related apoptosis-inducing ligand receptor 1 (TRAILR1; also known as TNFRSF10A), TRAILR2 (also known as TNFRSF10B) and receptor activator of nuclear factor-KB ligand (RANKL; also known as TNFSF11). Antigens involved in angiogenesis are usually proteins or growth factors that support the formation of new microvasculature, including vascular endothelial growth factor (VEGF), VEGF receptor (VEGFR), integrin αVβ3 and integrin α5β1. Tumor stroma and the extracellular matrix are indispensable support structures for a tumor. Stromal and extracellular matrix antigens that are therapeutic targets include fibroblast activation protein (FAP) and tenascin.

Examples of therapeutic antibodies useful in bispecific configurations or as combination therapy include, without limitation, rituximab; Ibritumomab; tiuxetan; tositumomab; Brentuximab; vedotin; Gemtuzumab; ozogamicin; Alemtuzumab; IGN101; adecatumumab; Labetuzumab; huA33; Pemtumomab; oregovomab; CC49 (minretumomab); cG250; J591; MOv18; MORAb-003 (farletuzumab); 3F8, ch14.18; KW-2871; hu3S193; IgN311; Bevacizumab; IM-2C6; CDP791; Etaracizumab; Volociximab; Cetuximab, panitumumab, nimotuzumab; 806; Trastuzumab; pertuzumab; MM-121; AMG 102, METMAB; SCH 900105; AVE1642, IMC-Al2, MK-0646, R1507; CP 751871; KB004; IIIA4; Mapatumumab (HGS-ETR1); HGS-ETR2; CS-1008; Denosumab; Sibrotuzumab; F19; and 81C6. A bispecific antibody may use the Fc region that activates an Fcγ receptor.

For the treatment of cancer, the anti-TREM-1 antibody may be combined with one or more antibodies that inhibit immune checkpoint proteins. Of particular interest are immune checkpoint proteins displayed on the surface of a tumor cell. The immune-checkpoint receptors that have been most actively studied in the context of clinical cancer immunotherapy, cytotoxic T-lymphocyte-associated antigen 4 (CTLA4; also known as CD152) and programmed cell death protein 1 (PD1; also known as CD279)—are both inhibitory receptors. The clinical activity of antibodies that block either of these receptors implies that antitumor immunity can be enhanced at multiple levels and that combinatorial strategies can be intelligently designed, guided by mechanistic considerations and preclinical models.

The two ligands for PD1 are PD1 ligand 1 (PDL1; also known as B7-H1 and CD274) and PDL2 (also known as B7-DC and CD273). PDL1 is expressed on cancer cells and through binding to its receptor PD1 on T cells it inhibits T cell activation/function. See, for example, Avelumab as a therapeutic antibody.

Agents that agonize an immune costimulatory molecule are also useful in the methods disclosed herein. Such agents include agonists or CD40 and OX40. CD40 is a costimulatory protein found on antigen presenting cells (APCs) and is required for their activation. These APCs include phagocytes (macrophages and dendritic cells) and B cells. CD40 is part of the TNF receptor family. The primary activating signaling molecules for CD40 are IFNγ and CD40 ligand (CD40L). Stimulation through CD40 activates macrophages.

Anti-CCR4 (CD194) antibodies of interest include humanized monoclonal antibodies directed against C-C chemokine receptor 4 (CCR4) with potential anti-inflammatory and antineoplastic activities.

In some embodiments, the additional therapeutic agent is an antibody that binds: HER2 (ERBB2/neu), CD52, PD-L1, VEGF, CD30, EGFR, CD38, RANKL (CD254), GD2 (ganglioside), SLAMF7 (CD319), CD20, EGFR, PDGFRa, VEGFR2, CD33, CD44, CD99, CD96, CD90, CD133, CKIT (CD117 for CKIT positive tumors); CTLA-4, PD-1, PD-L1, CD40 (agonistic), LAG3 (CD223), 41BB (CD137 agonistic), OX40 (CD134, agonistic); and/or CKIT (CD117) to deplete blood-forming stem cells for transplantation therapy.

In some embodiments, the additional therapeutic agent is at least one of: Rituximab, Cetuximab, Alemtuzumab (CD52), Atezolizumab (PD-L1), Avelumab (PD-L1), Bevacizumab (VEGF), Brentuximab (CD30), Daratumumab (CD38), Denosumab (RANKL), Dinutuximab (GD2), Elotuzumab (SLAMF7), Ibritumomab (CD20), Ipilimumab (CTLA-4), Necitumumab (EGFR), Nivolumab (PD-1), Obinutuzumab (CD20), Ofatumumab (CD20), Olaratumab (PDGFRa), Panitumumab (EGFR), Pembrolizumab (PD-1), Pertuzumab (HER2), Ramucirumab (VEGFR2), Tositumomab (CD20), and Gemtuzumab (CD33).

The additional therapeutic agent can be administered by any suitable means. In some embodiments, an antibody provided herein and the additional therapeutic agent are included in the same pharmaceutical composition. In some embodiments, an antibody provided herein and the additional therapeutic agent are included in different pharmaceutical compositions.

In embodiments where an antibody provided herein and the additional therapeutic agent are included in different pharmaceutical compositions, administration of the antibody can occur prior to, simultaneously, and/or following, administration of the additional therapeutic agent. In some aspects, administration of an antibody provided herein and the additional therapeutic agent occur within about one month of each other. In some aspects, administration of an antibody provided herein and the additional therapeutic agent occur within about one week of each other. In some aspects, administration of an antibody provided herein and the additional therapeutic agent occur within about one day of each other. In some aspects, administration of an antibody provided herein and the additional therapeutic agent occur within about twelve hours of each other. In some aspects, administration of an antibody provided herein and the additional therapeutic agent occur within about one hour of each other.

6.9. Pharmaceutical Compositions

An antibody provided herein can be formulated in any appropriate pharmaceutical composition and administered by any suitable route of administration. Suitable routes of administration include, but are not limited to, the intraarterial, intradermal, intramuscular, intraperitoneal, intravenous, nasal, parenteral, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a cosolvent. Illustrative examples of cosolvents include ethanol, poly(ethylene) glycol, butylene glycol, dimethylacetamide, glycerin, propylene glycol, and combinations thereof.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, monosodium glutamate, and combinations thereof.

In some embodiments, the pharmaceutical composition comprises a carrier or filler. Illustrative examples of carriers or fillers include lactose, maltodextrin, mannitol, sorbitol, chitosan, stearic acid, xanthan gum, guar gum, and combinations thereof.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, vitamin E polyethylene(glycol) succinate, and combinations thereof.

In some embodiments, the pharmaceutical composition comprises an anti-caking agent. Illustrative examples of anti-caking agents include calcium phosphate (tribasic), hydroxymethyl cellulose, hydroxypropyl cellulose, magnesium oxide, and combinations thereof.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, sugars, and combinations thereof. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising an antibody, since water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

In certain embodiments, an antibody provided herein is formulated as parenteral dosage forms. Parenteral dosage forms can be administered to subjects by various routes including, but not limited to, subcutaneous, intravenous (including infusions and bolus injections), intramuscular, and intra-arterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry (e.g., lyophilized) products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

In some embodiments, the parenteral dosage form is lyophilized. Exemplary lyophilized formulations are described, for example, in U.S. Pat. Nos. 6,267,958 and 6,171,586; and WO 2006/044908; each of which is incorporated by reference in its entirety.

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibody.

The amount of the antibody or composition which will be effective in the prevention or treatment of a disorder or one or more symptoms thereof will vary with the nature and severity of the disease or condition, and the route by which the antibody is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies provided herein are also encompassed by the dosage amounts and dose frequency schedules provided herein. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an antibody or composition provided herein followed by one or more maintenance doses.

In certain embodiments, a dose of an antibody or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

As discussed in more detail elsewhere in this disclosure, an antibody provided herein may optionally be administered with one or more additional agents useful to prevent or treat a disease or disorder. The effective amount of such additional agents may depend on the amount of antibody present in the formulation, the type of disorder or treatment, and the other factors known in the art or described herein.

6.10. Kits and Articles of Manufacture

The present application provides kits comprising any one or more of the antibody compositions described herein. In some embodiments, the kits further contain a component selected from any of secondary antibodies, reagents for immunohistochemistry analysis, pharmaceutically acceptable excipient and instruction manual and any combination thereof. In one specific embodiment, the kit comprises a pharmaceutical composition comprising any one or more of the antibody compositions described herein, with one or more pharmaceutically acceptable excipients.

In some embodiments, the kit comprises a container and a label or package insert on or associated with the container. Suitable containers include, for example, bottles, vials, syringes, and IV solution bags. The containers may be formed from a variety of materials, such as glass or plastic. The container holds a composition that is by itself, or when combined with another composition, effective for treating, preventing and/or diagnosing a disease or disorder. The container may have a sterile access port. For example, if the container is an intravenous solution bag or a vial, it may have a port that can be pierced by a needle. At least one active agent in the composition is an antibody provided herein. The label or package insert indicates that the composition is used for treating the selected condition.

In some embodiments, the kit comprises (a) a first container with a first composition contained therein, wherein the first composition comprises an antibody provided herein; and (b) a second container with a second composition contained therein, wherein the second composition comprises a further therapeutic agent. The kit in this embodiment can further comprise a package insert indicating that the compositions can be used to treat a particular condition.

Alternatively, or additionally, the kit may further comprise a second (or third) container comprising a pharmaceutically-acceptable excipient. In some aspects, the excipient is a buffer. The kit may further include other materials desirable from a commercial and user standpoint, including filters, needles, and syringes.

The present application also provides articles of manufacture comprising any one of the antibody compositions or kits described herein. Examples of an article of manufacture include vials (including sealed vials).

6.11. Assays

A variety of assays known in the art may be used to identify and characterize an TREM1 antibody provided herein.

### Binding, Competition, and Epitope Mapping Assays

Specific antigen-binding activity of an antibody provided herein may be evaluated by any suitable method, including using SPR, BLI, RIA and MSD-SET, as described elsewhere in this disclosure. Additionally, antigen-binding activity may be evaluated by ELISA assays and Western blot assays.

Assays for measuring competition between two antibodies, or an antibody and another molecule (e.g., one or more ligands of TREM1) are described elsewhere in this disclosure and, for example, in Harlow and Lane, Antibodies: A Laboratory Manual ch.14, 1988, Cold Spring Harbor Laboratory, Cold Spring Harbor, N.Y, incorporated by reference in its entirety.

Assays for mapping the epitopes to which an antibody provided herein bind are described, for example, in Morris “Epitope Mapping Protocols,” in Methods in Molecular Biology vol. 66, 1996, Humana Press, Totowa, N.J., incorporated by reference in its entirety. In some embodiments, the epitope is determined by peptide competition. In some embodiments, the epitope is determined by mass spectrometry. In some embodiments, the epitope is determined by crystallography.

### Assays for Effector Functions

Effector function following treatment with an antibody provided herein may be evaluated using a variety of in vitro and in vivo assays known in the art, including those described in Ravetch and Kinet, Annu. Rev. Immunol., 1991, 9:457-492; U.S. Pat. Nos. 5,500,362, 5,821,337; Hellstrom et al., Proc. Nat'l Acad. Sci. USA, 1986, 83:7059-7063; Hellstrom et al., Proc. Nat'l Acad. Sci. USA, 1985, 82:1499-1502; Bruggemann et al., J. Exp. Med., 1987, 166:1351-1361; Clynes et al., Proc. Nat'l Acad. Sci. USA, 1998, 95:652-656; WO 2006/029879; WO 2005/100402; Gazzano-Santoro et al., J. Immunol. Methods, 1996, 202:163-171; Cragg et al., Blood, 2003, 101:1045-1052; Cragg et al. Blood, 2004, 103:2738-2743; and Petkova et al., Int'l. Immunol., 2006, 18:1759-1769; each of which is incorporated by reference in its entirety.

An exemplary assay for antibody-dependent cell-mediated cytotoxicity (ADCC) is to use target cells expressing hCD16. An exemplary assay for antibody-mediated phagocytosis (ADCP) is to use target cells expressing hCD32. For both assays, HEK 293 parental or over-expressing human TREM-1 are plated in white flat bottom 96 well plates (Costar). Titrations of indicated antibodies are incubated with these cells within a range of 0 ug/ml to 10 ug/ml in a 1:4 or 1:5 dilution range across 8 points for 10 mins at room temperature. After incubation with antibodies, 75,000 Jurkat reporter cells over-expressing either hCD16 (BPS Biosciences) or hCD32 (BPS Biosciences) and NFAT responsive luciferase are added to labelled cells at a 3:1 effector to target ratio. The co-culture is incubated for 6 hours at 37° C. in a 5% CO₂ atmosphere. NFAT (luciferase) activity is measured by adding luciferase assay substrate and reagent to all wells (BPS Biosciences or Promega Corporation). Luminescence is read using a Spectramax plate reader (Molecular Devices). EC50 values are calculated by curve fitting luciferase signal generated from antibodies binding to over-expressing cells over background luminescence generated from HEK293 parental cells in Graphpad Prism (Graphpad Software).

ADCC is determined as loss of GFP positive target cells as a function of antibody concentration. The ability of antibodies themselves can also assessed for their ability to induce macrophage-independent ADCC. ADCP is determined as the formation of Cell-Trace Violet/GFP double positive cells gated on singlets as a function of antibody concentration. EC50 values are calculated by curve fitting ADCC/ADCP generated from antibodies binding to over-expressing cells over background generated from HEK293 parental cells in Graphpad Prism (Graphpad Software).

Additional exemplary assays for ADCC and ADCP include assays using primary cells, such as macrophages. Macrophages may be bone marrow derived macrophages (BMDM) or monocyte-derived macrophages (MDM). For the macrophage ADCC and ADCP assays, 50,000 GFP positive parental BW or BW over-expressing human TREM1, or GFP positive parental HEK 293 or HEK 293 over-expressing human TREM1 are plated in 96 well plates. Titrations of indicated unconjugated antibodies are incubated with these cells within a range of 0 ug/ml to 10 ug/ml in a 1:4 or 1:5 dilutions range at up to 8 points for 15 mins at room temperature. After incubation with antibodies, 50,000 Cell Trace Violet (Molecular Probes by Life Technologies) labelled IFN-γ (Peprotech) or LPS (Sigma-Aldrich)/IFN-γ treated macrophages are added to labelled target cells at a 1:1 effector to target ratio. Macrophages are treated with IFN-γ the day prior to harvest and treated with LPS 1 hr before harvest. The co-culture is incubated for 18 hours at 37° c. in a 5% CO₂ atmosphere, after which the culture is harvested and analyzed for ADCC and ADCP by flow cytometry (BD Fortessa X-14, BD Biosciences).

An exemplary assay for complement-dependent cytotoxicity (CDC) is to use target cells expressing human TREM1. Cell are harvested from culture in log phase and 5×10⁴-2×10⁵ cells are plated in 96 well U bottom plates. After plating, a 1:1 mixture of human complement containing serum and anti-human TREM1 mAbs (hIgG1 isotype) is added, and incubated with the target cells for 2-3 hrs at 37° C. After incubation, antibody-mediated CDC activity is assessed by measuring target cell death by flow cytometry or luminescence-based methods such as Cell Titer Glo (Promega, Madison, WI).

### 7. EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3r d Ed. (Plenum Press) Vols A and B(1992).

### 7.1. Example 1: Characterization of Anti-mTREM1 Antibodies

### Epitope Binning Experiments:

100,000 HEK293 cells over-expressing mouse TREM-1 were plated in 96 well plates and dead cells were stained with Zombie Near Infrared (Biolegend, San Diego, CA). These cells were subsequently incubated with the indicated unconjugated anti-mouse TREM-1 antibodies at saturating concentrations for 15 mins on ice. Following incubation with these antibodies, cells were then stained with the indicated Alexa Fluor 647 conjugated antibodies for 30 mins on ice. The ability of conjugated antibodies to bind in the presence of unconjugated antibodies was assessed by Alexa 647 signal measured by flow cytometry (BD Fortessa X-14, BD Biosciences, San Jose, CA). Each antibody was validated to block itself for an internal experimental control.

### Source of Antibodies:

PI-9067s, PI-9067L, PI-9068, PI-9069s, and PI-9069L were made by Pionyr Immunotherapeutics. Sequences for the V_{L}, V_{H}, C_{L}, C_{H}, and CDRs are shown in Table 2. Anti-TREM1 clone L5-B8.2Al2.3Al2 was purchased from Thermo (ThermoFisher Scientific, Waltham, MA). Anti-TREM1 clone 174031 was purchased from R&D (R&D Systems, Minneapolis, MN). Anti-TREM1 clone TR3MBL1 was purchased from eBio (eBioscience, Carlsbad, CA).

PI-9067s and PI-9069s have a fully human kappa light chain, and a fully human heavy chain up to the end of the hinge region Immediately after this, the remaining CH2 and CH3 domains of the Fc are mouse. PI-9067L and PI-9069L have a fully mouse constant sequence which starts immediately after the human variable regions of both the heavy and light chains. The antibodies were re-engineered from the “s” sequences to the “L” sequences to improve the pharmacokinetic properties of the antibodies and to improve the geometry and flexibility of the Fc portion of the mAb by having a fully mouse hinge and Fc region.

### Protein Binding (Kd Measurement):

Binding kinetics were determined by surface plasmon resonance using a PROTEON XPR36 (BioRad, Hercules, CA) or Biacore T200 (GE Healthcare, UK) with mouse TREM-1 His (Creative Biomart, Shirley, NY) immobilized or captured on GLC or Series S CMS chips. Serial dilutions of indicated antibodies were injected at 50-100 ul/minute for 2 minutes. PBS or system buffer was then injected at 100 ul/minute for 10 minutes to observe dissociation. Binding responses were corrected by subtraction of responses on a blank flow cell. For kinetic analysis, a 1:1 Langmuir model of global fittings of kₒₙ and k_{off} values, was used. The K_{d} values were determined from the ratios of kₒₙ and k_{off}.

### Cellular Binding (EC50 Measurement):

100,000 HEK 293 parental or over-expressing mouse TREM-1 were plated in 96 well plates and dead cells were stained with Zombie Near Infrared (Biolegend, San Diego, CA). Titrations of indicated unconjugated antibodies were incubated with these cells within a range of 0 ug/ml to 30 ug/ml in a 1:4 or 1:5 dilution range across 8 points. Dependent on their isotype (hIgG1 or mIgG2a), these primary unconjugated antibodies were detected with Alexa Fluor 647 conjugated anti-human Fcγ or anti-mouse Fcγ secondary antibodies (Jackson Immunoresearch, West Grove, PA). Alexa Fluor 647 signal was measured by flow cytometry (BD Fortessa X-14, BD Biosciences, San Jose, CA). EC50 values were calculated by curve fitting signal generated from antibodies binding to over-expressing cells over background fluorescence generated from HEK293 parental cells in Graphpad Prism (Graphpad Software, La Jolla, CA).

### ADCC and ADCP Reporter Assays (EC50 Measurement):

25,000 HEK 293 parental or over-expressing mouse TREM-1 were plated in white flat bottom 96 well plates (Costar, Corning, NY). Titrations of indicated unconjugated antibodies (hIgG1 or mIgG2a) were incubated with these cells within a range of 0 ug/ml to 10 ug/ml in a 1:4 or 1:5 dilution range across 8 points for 10 mins at room temperature. After incubation with antibodies, 75,000 Jurkat reporter cells over-expressing either hCD32 (for hIgG1 isotype, BPS Biosciences, San Diego, CA) or mFcγRIV (for mIgG2a isotype, Promega Corporation, Madison, WI) and NFAT responsive luciferase were added to labelled cells at a 3:1 effector to target ratio. The coculture was incubated for 6 hours at 37° c. in a 5% CO₂ atmosphere. NFAT (luciferase) activity was measured by adding luciferase assay substrate and reagent to all wells (BPS Biosciences or Promega Corporation). Luminescence was read using a Spectramax plate reader (Molecular Devices, Sunnyvale, CA). EC50 values were calculated by curve fitting luciferase signal generated from antibodies binding to over-expressing cells over background luminescence generated from HEK293 parental cells in Graphpad Prism (Graphpad Software).

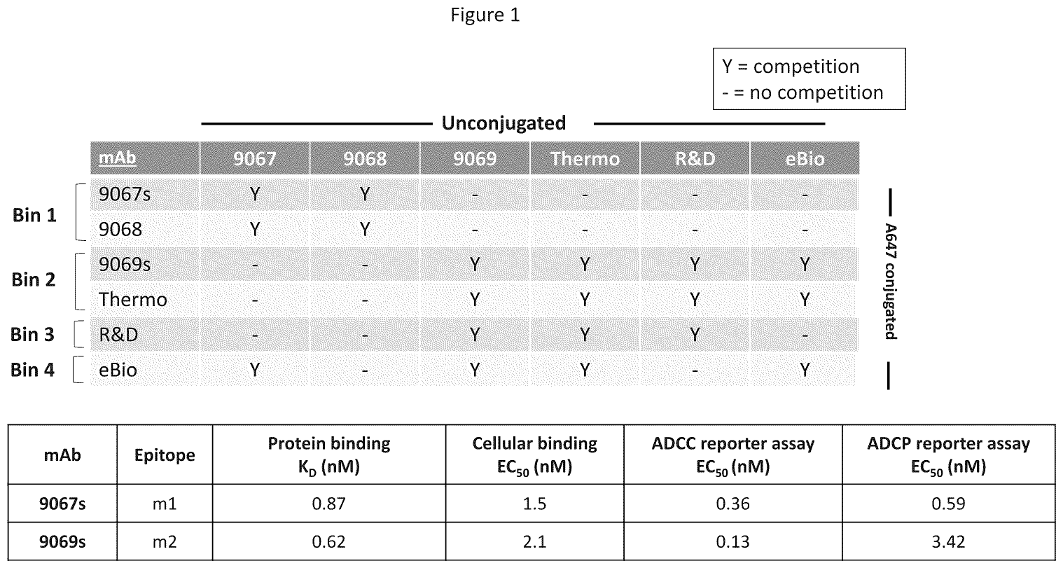

FIG. 1 shows the results of recombinant and cell-based specific TREM1 binding, epitope binning, and ADCC/ADCP functional experiments with anti-mTREM1 antibodies. This data shows the generation of high affinity anti-mouse TREM1 antibodies, from a range of epitopes, which are able to elicit ADCC and ADCP activity.

### 7.2. Example 2: MC38 In Vivo Efficacy Experiment

MC38 (colon adenocarcinoma cells) were harvested using StemPro Accutase Cell Dissociation reagent (Gibco Corporation, ThermoFisher Scientific, Waltham, MA), washed in PBS and subcutaneously injected into female C57BL/6 mice (Jackson Laboratories, Bar Harbor, ME) at a dose of 8×10⁵ cells in the right ventral flank. Tumor growth was monitored until the average size of tumors was between 75-130 mm³ and mice were subsequently randomized into 3 treatment groups representing an isotype control (mIgG2a), and two anti-mouse TREM1 clones designated Pi-9067s and Pi-9069s. Antibody treatments were all 15 mg/kg and treatments were Day 9, 14, 18, and 23 post tumor inoculation. Tumor size was assessed using the formula V (mm³)=(LxWxW)÷2; where W=width, L=length and V=volume. Tumors were measured twice to three times weekly using calipers and mice were euthanized when tumor volume reached 2000 mm³.

### Source of Antibodies:

mIgG2a isotype control clone C1.18.4 was purchased from BioXCell (BioXCell, West Lebanon, NH). Pi-9067s and Pi-9069s were made by Pionyr Immunotherapeutics. Sequences are shown in Table 2.

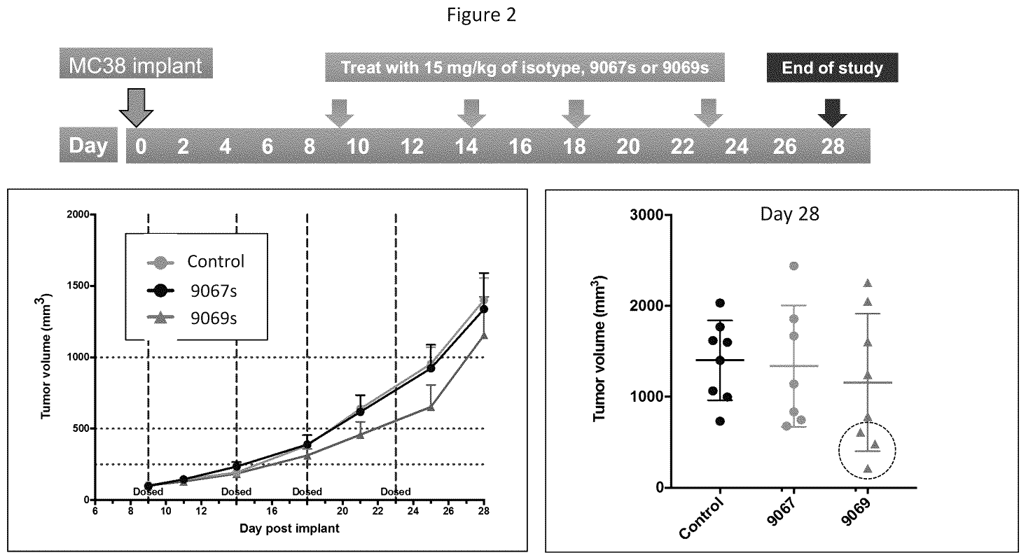

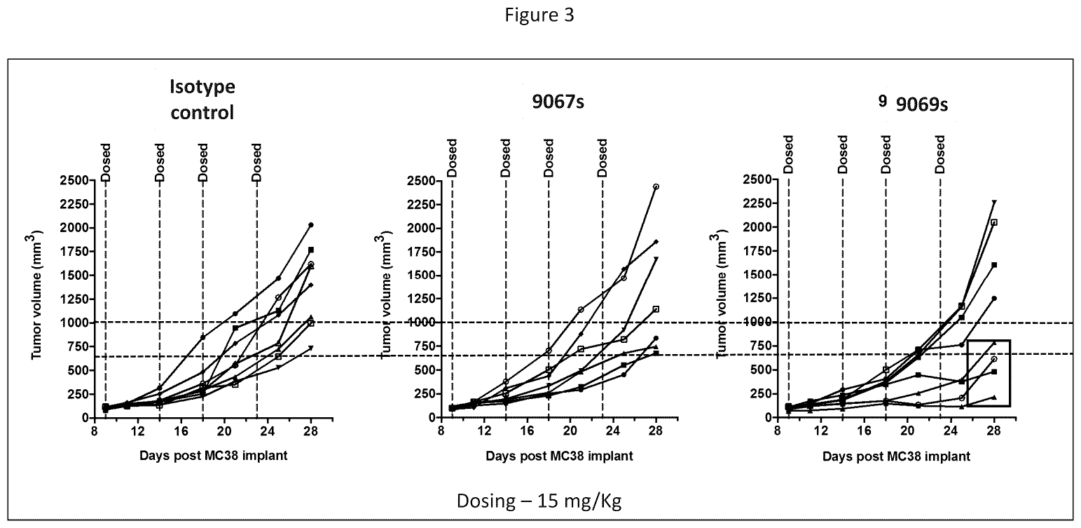

FIG. 2 shows the anti-tumor activity of 9069s treatment as a monotherapy in the MC38 model compared to isotype control and 9067s. FIG. 3 shows the responses of individual MC38 tumor-bearing mice to isotype, 9067s and 9069s monotherapy treatment. PI-9069s demonstrated stronger anti-tumor activity than PI-9067s in the MC38 tumor model.

### 7.3. Example 3: CT26 In Vivo Efficacy Experiment

CT26 (colon adenocarcinoma cells) were harvested using StemPro Accutase Dissociation reagent (Gibco Corporation), washed in PBS and subcutaneously injected into female BALB/c mice (Taconic Biosciences, Hudson, NY) at a dose of 1×10⁶ cells in the right ventral flank. Tumor growth was monitored until the average size of tumors was between 75-130 mm³ and mice were subsequently randomized into 4 treatment groups representing isotype treatment (mIgG2a and mIgG1), single agent anti-mouse TREM-1 (Pi-9069L and mIgG1), single agent anti-mouse PD-1 (mIgG2a and Pi-7114), and combination treatment (Pi-9069L and Pi-7114). Antibody treatments for mIgG2a and Pi-9069L were 15 mg/kg and for mIgG1 and Pi-7114 were 5 mg/kg. Treatments were Day 7, 12, 17, and 22 post tumor inoculation. Tumor size was assessed using the formula V (mm³)=(LxWxW)÷2; where W=width, L=length and V=volume. Tumors were measured twice to three times weekly using calipers and mice were euthanized when tumor volume reached 2000 mm³.

### Source of Antibodies:

mIgG1 isotype control clone MOPC-21 was purchased from BioXCell (BioXCell, West Lebanon, NH). mIgG2a isotype control clone C1.18.4 was purchased from BioXCell (BioXCell, West Lebanon, NH). Pi-9069L and Pi-7114 were made by Pionyr Immunotherapeutics. Pi-7114 is clone RMP1-14 (BioXCell, West Lebanon, NH) isotype switched from rIgG2a to mIgG1.

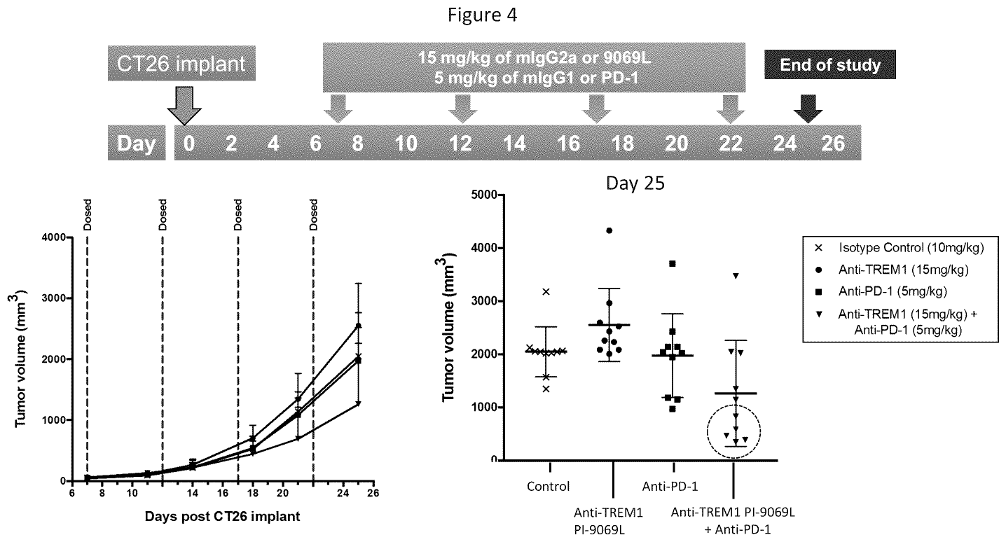

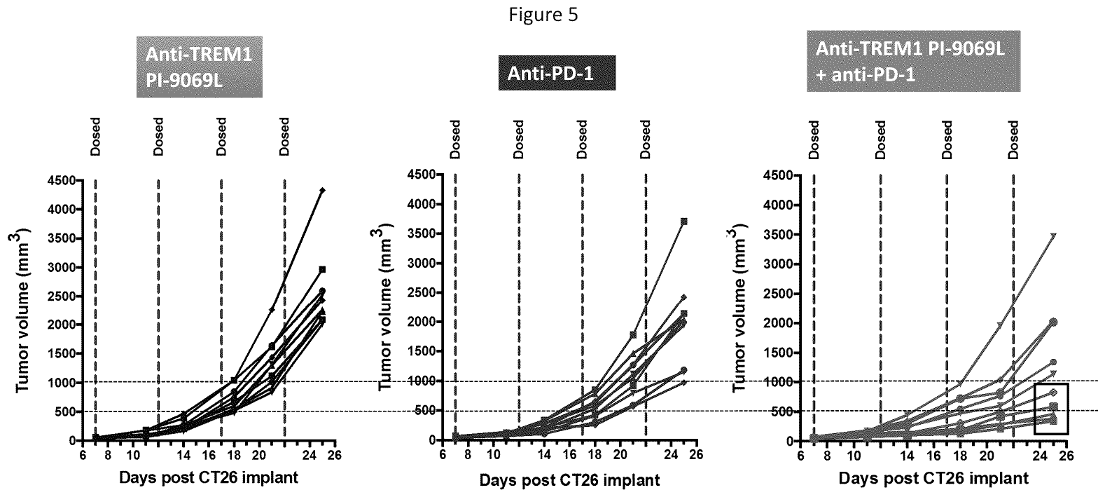

FIG. 4 shows anti-tumor activity of 9069L and anti-PD1 combination treatment in the CT26 model relative to controls. FIG. 5 shows the responses of individual CT26 tumor-bearing mice to combination treatment with 9069L and anti-PD1.

The combination of 9069L and anti-PD-1 showed stronger anti-tumor efficacy than 9069L alone, anti-PD-1 alone, and isotype treated mice. This effect could be seen in greater detail when analyzing the growth curves of individual mice. In the combination group, there were a subset of strong responders which were absent in the single agent control groups.

### 7.4. Example 4: Receptor Occupancy (RO) and Pharmacodynamic (PD) Studies

CT26 (colon adenocarcinoma), LL/2 (lung adenocarcinoma), MC38 (colon adenocarcinoma), and RENCA (renal cell carcinoma) were harvested using StemPro Accutase Dissociation reagent (Gibco Corporation), washed in PBS and subcutaneously injected to female C57BL/6 (Jackson Laboratories) or BALB/c (Taconic Biosciences) mice at a dose between 8×10⁵-1×10⁶ cells in the right ventral flank. Tumor growth was monitored until the average size of tumors was between 75-130 mm³ and mice were subsequently randomized into 2 treatment groups representing isotype treatment (mIgG2a) and anti-mouse TREM1 (Pi-9069s or L). Antibody treatments were 15 mg/kg for both arms and mice were treated twice at the indicated time points. 48 hr after the second dose, mice were euthanized and the tumor and blood were harvested for immune population analysis and expression of TREM1 Immune population and expression analyses were performed by flow cytometry (BD Fortessa X-14, BD Biosciences).

Monocyte and TAM depletion scores were calculated as the ratio of monocytes or TAMs to CD103+ DC in the isotype treated group relative to the ratio of monocytes or TAMs to CD103+ DC in the 9069s or L treated group. Higher depletion scores indicate more depletion of TAMs or monocytes relative to CD103+ DC.

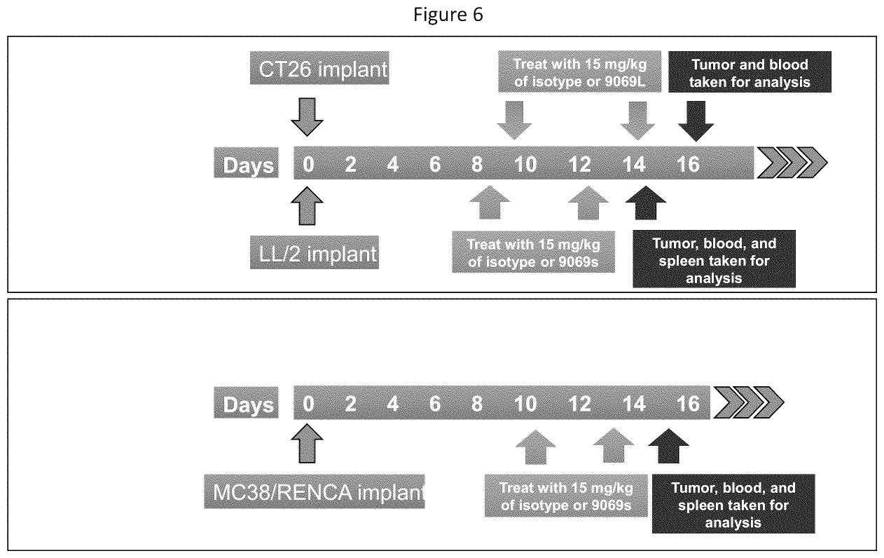

FIG. 6 shows a schematic of the experimental design of intratumoral receptor occupancy and pharmacodynamics studies with 9069s or L in four distinct syngeneic tumor models.

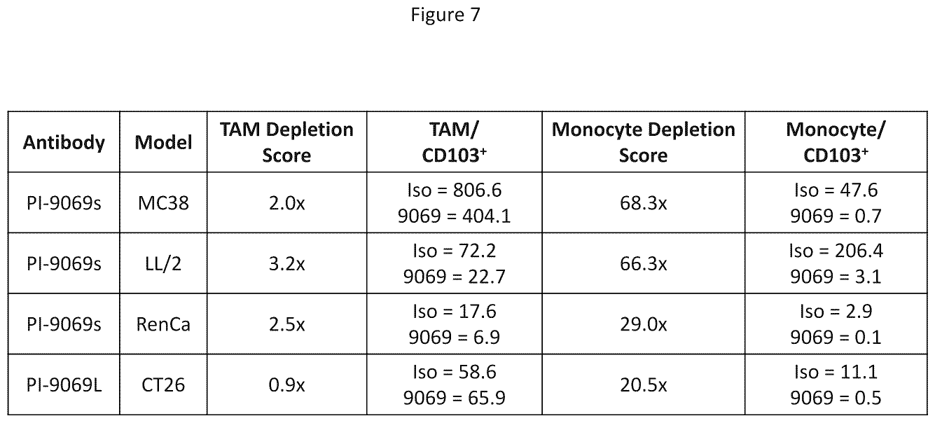

FIG. 7 shows that 9069s or L increases intratumoral TAM and Monocyte depletion scores in four distinct syngeneic tumor models. In particular, RenCa and LL/2 were good tumor models to test anti-TREM1 efficacy.

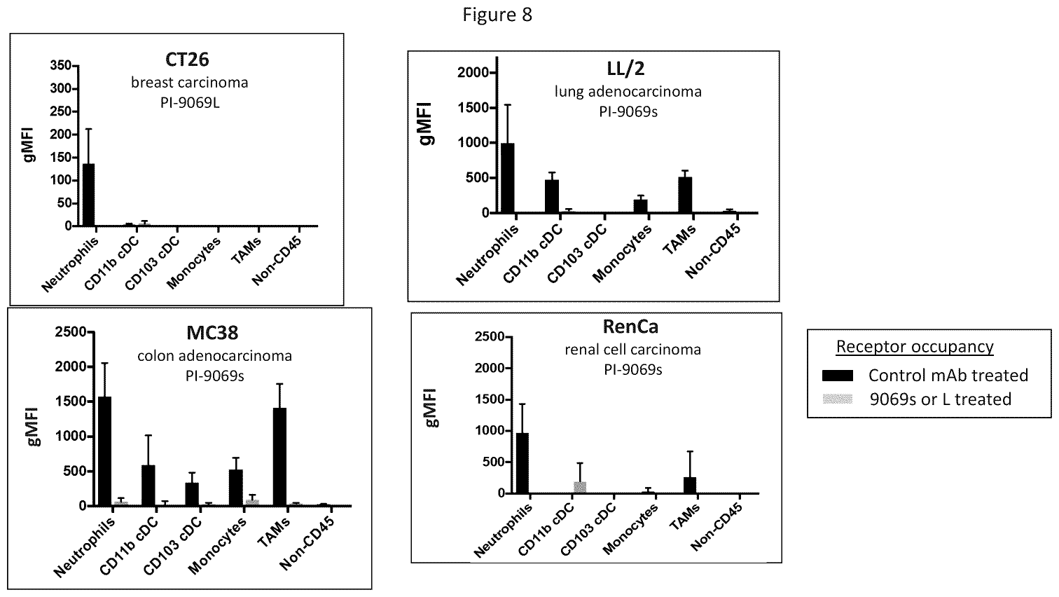

FIG. 8 shows that 9069s or L demonstrates intratumoral myeloid-specific RO activity in four distinct syngeneic tumor models.

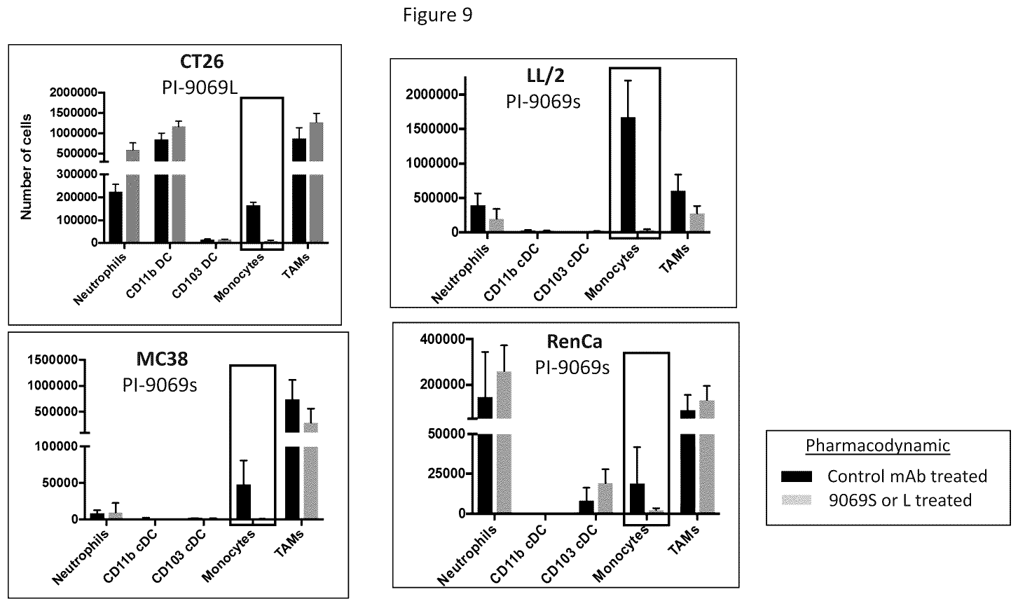

FIG. 9 shows that 9069s or L demonstrates intratumoral myeloid-specific PD activity in four distinct syngeneic tumor models.

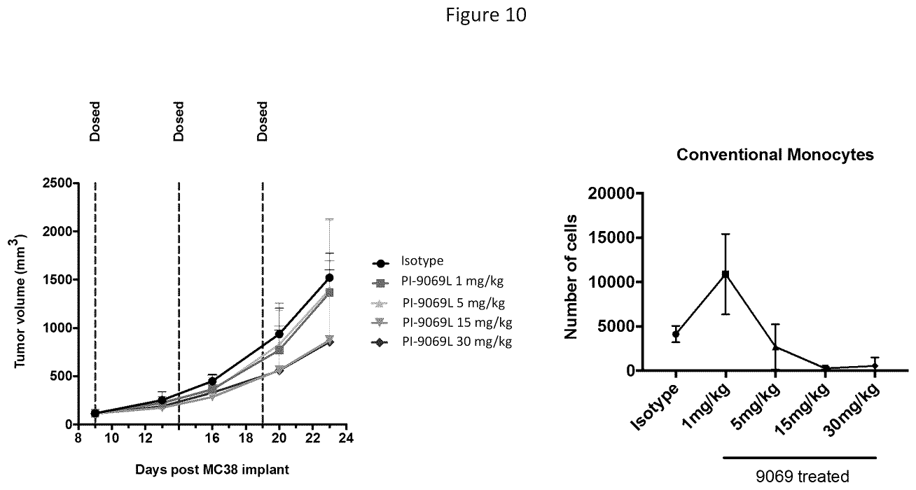

FIG. 10 shows a dose escalation efficacy experiment of 9069L in the MC38 model and that efficacy correlates with depletion of intratumoral monocytes.

Naïve mice C57BL/6 mice (Jackson Laboratories) were treated two doses of mIgG2a isotype control or 9069L at 15 mg/kg per dose. 48 hrs after the second dose, mice were euthanized and the blood and bone marrow were harvested for monocyte and neutrophil analysis, as well as expression of TREM1. Immune population and expression analyses were performed by flow cytometry (BD Fortessa X-14, BD Biosciences).

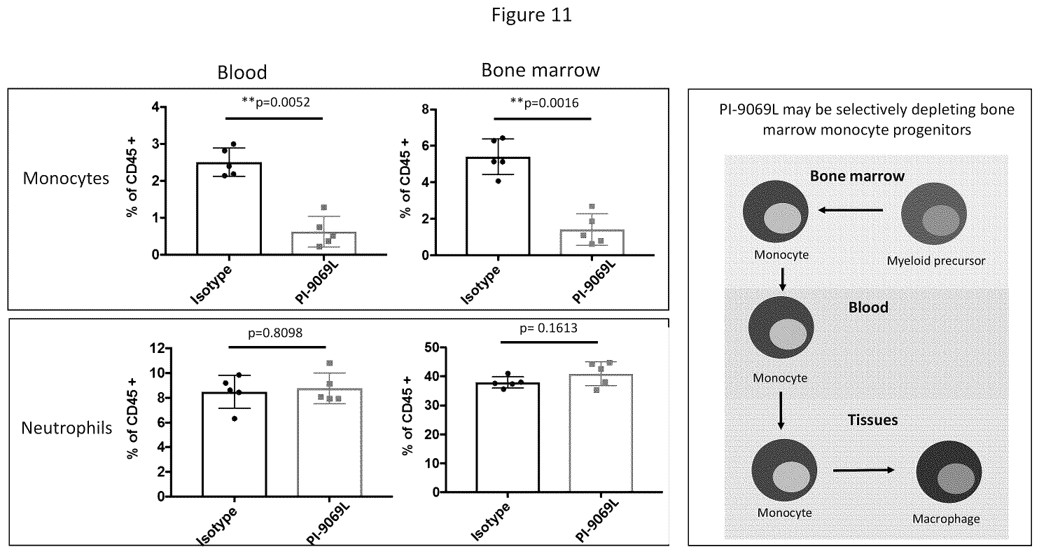

FIG. 11 shows that 9069L significantly reduces monocytes, but not neutrophils, in the blood and bone marrow of naïve mice and may be depleting monocyte progenitors.

In summary, FIGS. 6-11 show that PI-9069s or L adequately enters the tumor microenvironment in 4 distinct models and is able to bind to TREM1-expressing myeloid populations. Furthermore, PI-9069s or L reduced the number of TREM1-expressing cell populations, particularly monocytes, in the tumor microenvironment that correlates with anti-tumor activity in a dose-dependent manner.

### 7.5. Example 5: Characterization of Anti-hTREM1 mAbs and TREM1 Expression in Primary Human Tumor Samples

### Epitope Binning Experiments:

100,000 HEK293 cells over-expressing human TREM-1 were plated in 96 well plates and dead cells were stained with Zombie Near Infrared (Biolegend). These cells were subsequently incubated with the indicated unconjugated anti-human TREM-1 antibodies at saturating concentrations for 15 mins on ice. Following incubation with these antibodies, cells were then stained with the indicated Alexa Fluor 647 conjugated antibodies for 30 mins on ice. The ability of conjugated antibodies to bind in the presence of unconjugated antibodies was assessed by Alexa 647 signal measured by flow cytometry (BD Fortessa X-14, BD Biosciences). Each antibody was validated to block itself for an internal experimental control.

### Source of Antibodies:

Pi-8419 and Pi-8421 were made by Pionyr Immunotherapeutics. Sequences for the V_{L}, V_{H}, and CDRs are shown in Table 2. mAb0170: Sequence obtained from Patent US2013/0211050 A1 and engineered by Pionyr Immunotherapeutics. Sequences for the V_{L}, V_{H}, and CDRs are shown in Table 2.

### Cellular Binding (EC50 Measurement):

100,000 HEK 293 parental or over-expressing human or cynomolgus TREM-1 were plated in 96 well plates and dead cells were stained with Zombie Near Infrared (Biolegend). Titrations of indicated unconjugated antibodies were incubated with these cells within a range of 0 ug/ml to 30 ug/ml in a 1:4 or 1:5 dilution range across 8 points. Dependent on their isotype (hIgG1 or mIgG2a), these primary unconjugated antibodies were detected with Alexa Fluor 647 conjugated anti-human Fcγ or anti-mouse Fcγ secondary antibodies (Jackson Immunoresearch). Alexa Fluor 647 signal was measured by flow cytometry (BD Fortessa X-14, BD Biosciences). EC50 values were calculated by curve fitting signal generated from antibodies binding to over-expressing cells over background fluorescence generated from HEK293 parental cells in Graphpad Prism (Graphpad Software).

### ADCC and ADCP Reporter Assays (EC50 Measurement)

25,000 HEK 293 parental or over-expressing human TREM-1 were plated in white flat bottom 96 well plates (Costar). Titrations of indicated unconjugated antibodies (hIgG1) were incubated with these cells within a range of 0 ug/ml to 10 ug/ml in a 1:4 or 1:5 dilution range across 8 points for 10 mins at room temperature. After incubation with antibodies, 75,000 Jurkat reporter cells over-expressing either hCD32 (BPS Biosciences) or hCD16 (BPS Biosciences) and NFAT responsive luciferase were added to labelled cells at a 3:1 effector to target ratio. The co-culture was incubated for 6 hours at 37° c. in a 5% CO₂ atmosphere. NFAT (luciferase) activity was measured by adding luciferase assay substrate and reagent to all wells (BPS Biosciences or Promega Corporation). Luminescence was read using a Spectramax plate reader (Molecular Devices). EC50 values were calculated by curve fitting luciferase signal generated from antibodies binding to over-expressing cells over background luminescence generated from HEK293 parental cells in Graphpad Prism (Graphpad Software).

### ADCC/ADCP (Primary Monocyte Derived Macrophage/MDM Assay):

Monocytes were isolated using negative selection for CD14+ cells (StemCell Technologies, Vancouver, Canada) from PBMC that had undergone Ficoll (GE Healthcare) isolation from peripheral blood from healthy donors (Stanford Blood Centre, Palo Alto, CA). Monocytes were plated in non-TC treated 15 cm dishes or 6 well plates with 50 ng/ml human M-CSF (R&D systems, Minneapolis, MN) in macrophage media. Macrophage media consisted of RPMI (Gibco), 10% FCS (Hyclone, GE Healthcare), Glutamax (Gibco), Non-essential amino acids (Gibco), Sodium pyruvate (Gibco), 2-betamercaptoethanol (Gibco), and penicillin/streptomycin (University of California, San Francisco). Media was replenished at Day 3 and monocytes had differentiated to macrophages and were ready to use between 6-8 days post monocyte isolation.

For the MDM assay, 50,000 GFP positive parental BW or BW over-expressing human TREM1, or GFP positive parental HEK 293 or HEK 293 over-expressing human TREM1 were plated in 96 well plates. Titrations of indicated unconjugated antibodies were in incubated with these cells within a range of 0 ug/ml to 10 ug/ml in a 1:4 or 1:5 dilutions range at up to 8 points for 15 mins at room temperature. After incubation with antibodies, 50,000 Cell Trace Violet (Molecular Probes by Life Technologies, ThermoFisher Scientific) labelled IFN-γ (Peprotech, Rocky Hill, NJ) or LPS (Sigma-Aldrich, St Louis, MO)/IFN-γ treated macrophages were added to labelled target cells at a 1:1 effector to target ratio. Macrophages were treated with IFN-γ the day prior to harvest and treated with LPS 1 hr before harvest. The co-culture was incubated for 18 hours at 37° C. in a 5% CO₂ atmosphere, after which the culture was harvested and analyzed for ADCC and ADCP by flow cytometry (BD Fortessa X-14, BD Biosciences).

ADCC was determined as loss of GFP positive target cells as a function of antibody concentration. The ability of antibodies themselves were also assessed for their ability to induce macrophage-independent ADCC. ADCP was determined as the formation of Cell-Trace Violet/GFP double positive cells gated on singlets as a function of antibody concentration. EC50 values were calculated by curve fitting ADCC/ADCP generated from antibodies binding to over-expressing cells over background generated from HEK293 parental cells in Graphpad Prism (Graphpad Software).

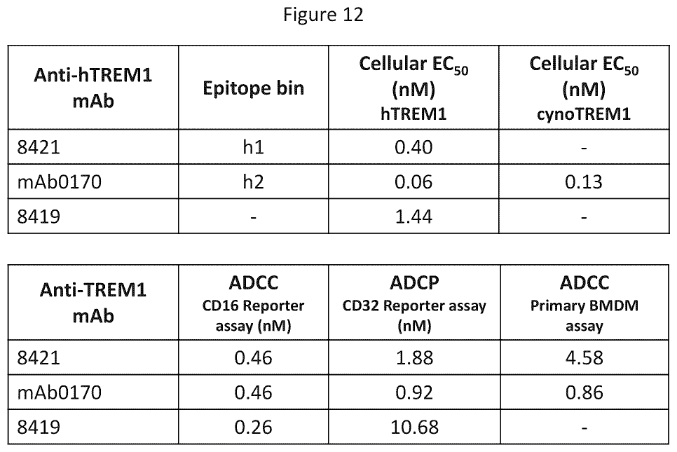

FIG. 12 shows the results of cell-based specific TREM1 binding, epitope binning, and ADCC/ADCP functional experiments with anti-hTREM1 antibodies. This data shows the generation of high affinity anti-human TREM1 antibodies, targeting a range of epitopes, which are able to elicit ADCC and ADCP activity.

Human tumor samples and normal adjacent tissue were sourced from the Cooperative Human Tissue Network (CHTN). Samples were digested into single cell suspensions using the Miltenyi Human Tumor Dissociation Kit (Miltenyi Biotec, Cologne, Germany). Single cell suspensions were plated into 96 well plates and infiltrating immune populations as well as TREM1 and TREM2 expression were analysed by flow cytometry (BD Fortessa X-14, BD Biosciences). Numbers indicate the level of specific TREM1 and TREM2 staining over isotype on specific immune populations from individual tumors.

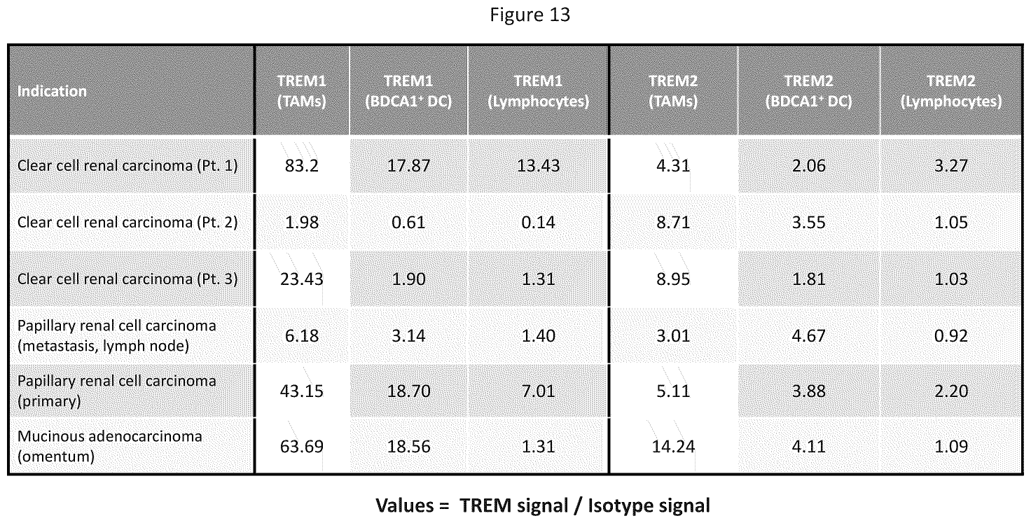

FIG. 13 shows that TREM1 is highly expressed on TAMs relative to DC and lymphocytes from primary human tumor samples. This data shows that TREM1 is expressed in the tumor microenvironment from human patients with cancer, and that TREM1 is highly expressed on myeloid populations, particularly TAMs.

### 7.6. Example 6: Epitope Characterization of Anti-TREM1 Antibodies

### Epitope Binning Experiments:

100,000 HEK293 cells over-expressing human TREM-1 were plated in 96 well plates and dead cells were stained with Zombie Near Infrared (Biolegend). These cells were subsequently incubated with the indicated unconjugated anti-human TREM-1 antibodies at saturating concentrations for 15 mins on ice. Following incubation with these antibodies, cells were then stained with the indicated Alexa Fluor 647 conjugated antibodies for 30 mins on ice. The ability of conjugated antibodies to bind in the presence of unconjugated antibodies was assessed by Alexa 647 signal measured by flow cytometry (BD Fortessa X-14, BD Biosciences). Each antibody was validated to block itself for an internal experimental control.

### Source of Antibodies:

mAb0170: Sequences obtained from Patent US2013/0211050 A1 and engineered by Pionyr Immunotherapeutics. Pi-8419 and Pi-8421 were made by Pionyr Immunotherapeutics. Sequences for the V_{L}, V_{H}, and CDRs are shown in Table 2. Anti-TREM1 clone 193015 was purchased from R&D (R&D systems, Minneapolis, MN)

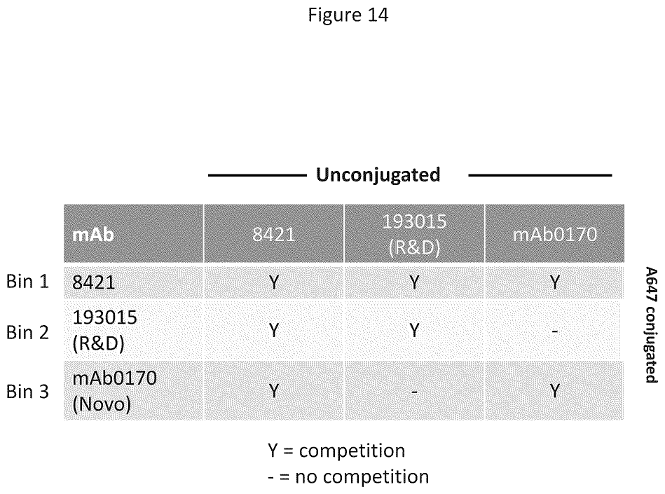

FIG. 14 shows the results of the binning experiments with anti-hTREM1 antibodies. PI-8421 displayed differential binding competition to both mAb0170 and 193015; indicating that it binds to a different epitope of human TREM1.

### 7.7. Example 7: Characterization of Pi 8421

### Cellular Binding (EC50 Measurement):

100,000 HEK 293 parental or over-expressing human or cynomolgus TREM-1 were plated in 96 well plates and dead cells were stained with Zombie Near Infrared (Biolegend). Titrations of indicated unconjugated antibodies were incubated with these cells within a range of 0 ug/ml to 30 ug/ml in a 1:4 or 1:5 dilution range across 8 points. Dependent on their isotype (hIgG1 or mIgG2a), these primary unconjugated antibodies were detected with Alexa Fluor 647 conjugated anti-human Fcγ or anti-mouse Fcγ secondary antibodies (Jackson Immunoresearch). Alexa Fluor 647 signal was measured by flow cytometry (BD Fortessa X-14, BD Biosciences). EC50 values were calculated by curve fitting signal generated from antibodies binding to over-expressing cells over background fluorescence generated from HEK293 parental cells in Graphpad Prism (Graphpad Software).

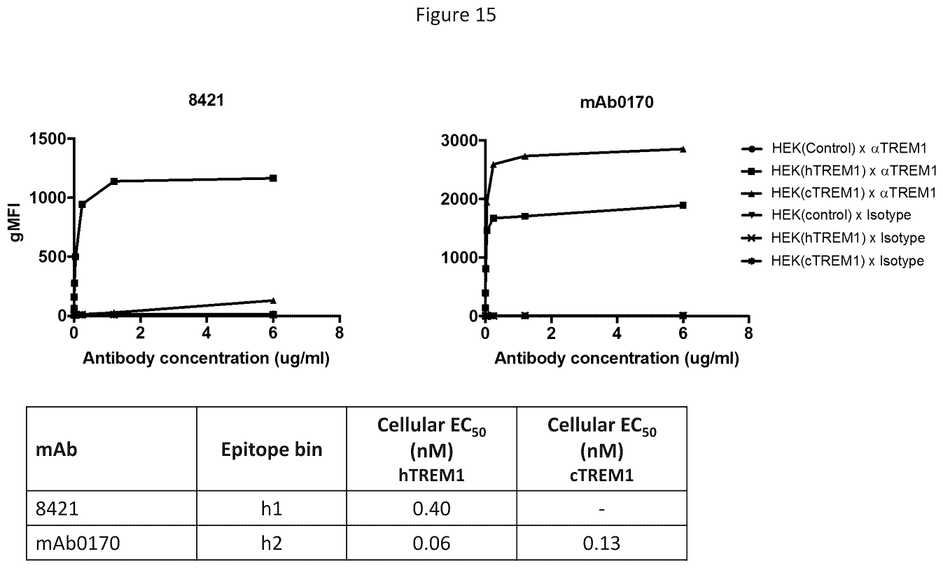

FIG. 15 shows that 8421 binds to hTREM1 but not cTREM1.

### 7.8. Example 8: ADCC and ADCP Activity of Pi 8421 and mAb0170

### ADCC and ADCP Reporter Assays (EC50 Measurement):

25,000 HEK 293 parental or over-expressing human TREM-1 were plated in white flat bottom 96 well plates (Costar). Titrations of indicated unconjugated antibodies (hIgG1) were incubated with these cells within a range of 0 ug/ml to 10 ug/ml in a 1:4 or 1:5 dilution range across 8 points for 10 mins at room temperature. After incubation with antibodies, 75,000 Jurkat reporter cells over-expressing either hCD32 (BPS Biosciences) or hCD16 (BPS Biosciences) and NFAT responsive luciferase were added to labelled cells at a 3:1 effector to target ratio. The co-culture was incubated for 6 hours at 37° c. in a 5% CO₂ atmosphere. NFAT (luciferase) activity was measured by adding luciferase assay substrate and reagent to all wells (BPS Biosciences or Promega Corporation). Luminescence was read using a Spectramax plate reader (Molecular Devices). EC50 values were calculated by curve fitting luciferase signal generated from antibodies binding to over-expressing cells over background luminescence generated from HEK293 parental cells in Graphpad Prism (Graphpad Software).

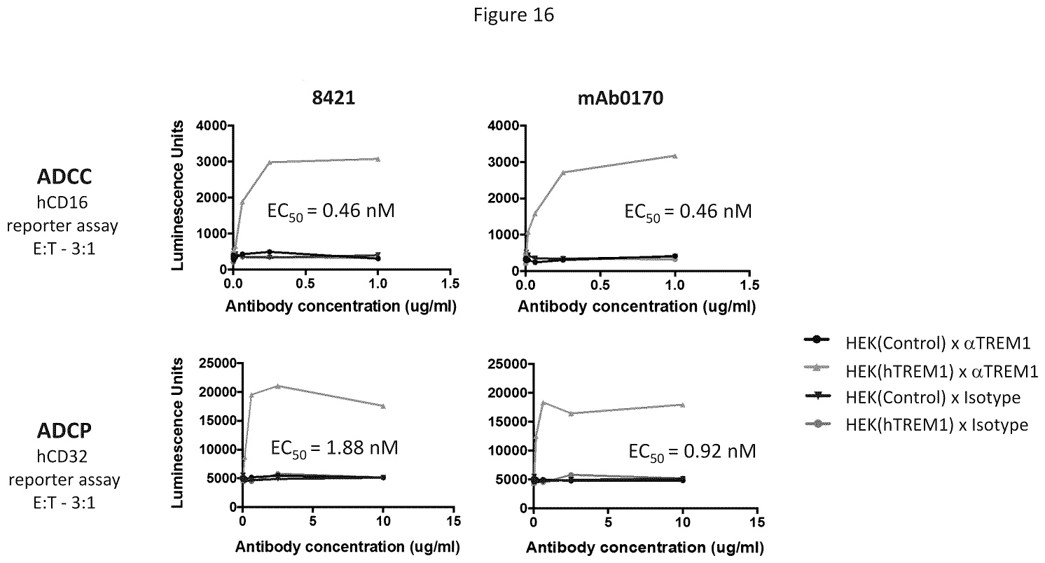

FIG. 16 shows that 8421 and 0170 have similar ADCC and ADCP activities in the reporter assay system tested. This data shows that PI-8421 has the ability to induce ADCC and ADCP in a TREM-1 dependent manner.

### 7.9. Example 9: ADCC and ADCP Activity of 8421 with Primary Macrophages

### ADCC/ADCP (Primary Monocyte Derived Macrophage/MDM Assay):

Monocytes were isolated using negative selection for CD14+ cells (StemCell Technologies, Vancouver, Canada) from PBMC that had undergone Ficoll (GE Healthcare) isolation from peripheral blood from healthy donors (Stanford Blood Centre, Palo Alto, CA). Monocytes were plated in non-TC treated 15 cm dishes or 6 well plates with 50 ng/ml human M-CSF (R&D systems) in macrophage media. Macrophage media consisted of RPMI (Gibco), 10% FCS (Hyclone, GE Healthcare), Glutamax (Gibco), Non-essential amino acids (Gibco), Sodium pyruvate (Gibco), 2-betamercaptoethanol (Gibco), and penicillin/streptomycin (University of California, San Francisco). Media was replenished at Day 3 and monocytes had differentiated to macrophages and were ready to use between 6-8 days post monocyte isolation.

For the MDM assay, 50,000 GFP positive parental BW or BW over-expressing human TREM1, or GFP positive parental HEK 293 or HEK 293 over-expressing human TREM1 were plated in 96 well plates. Titrations of indicated unconjugated antibodies were in incubated with these cells within a range of 0 ug/ml to 10 ug/ml in a 1:4 or 1:5 dilutions range at up to 8 points for 15 mins at room temperature. After incubation with antibodies, 50,000 Cell Trace Violet (Molecular Probes by Life Technologies) labelled IFN-γ (Peprotech) or LPS (Sigma-Aldrich)/IFN-γ treated macrophages were added to labelled target cells at a 1:1 effector to target ratio. Macrophages were treated with IFN-γ the day prior to harvest and treated with LPS 1 hr before harvest. The co-culture was incubated for 18 hours at 37° c. in a 5% CO₂ atmosphere, after which the culture was harvested and analyzed for ADCC and ADCP by flow cytometry (BD Fortessa X-14, BD Biosciences).

ADCC was determined as loss of GFP positive target cells as a function of antibody concentration. The ability of antibodies themselves were also assessed for their ability to induce macrophage-independent ADCC. ADCP was determined as the formation of Cell-Trace Violet/GFP double positive cells gated on singlets as a function of antibody concentration. EC50 values were calculated by curve fitting ADCC/ADCP generated from antibodies binding to over-expressing cells over background generated from HEK293 parental cells in Graphpad Prism (Graphpad Software).

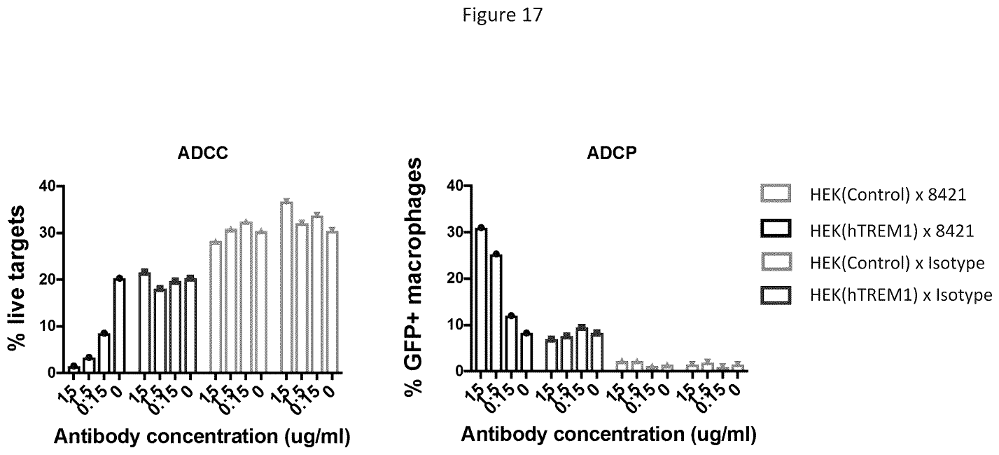

FIG. 17 shows that 8421 induces ADCC and ADCP activity in a primary macrophage setting. In summary, PI-8421 was able to elicit both ADCC and ADCP by primary macrophages in a TREM1-dependent manner.

### 7.10. Example 10: Characterization of Anti-mTREM1 Antibodies 9067L, 4928, and 9772

### Epitope Binning Experiments:

100,000 HEK293 cells over-expressing mouse TREM-1 were plated in 96 well plates and dead cells were stained with Zombie Near Infrared (Biolegend, San Diego, CA). These cells were subsequently incubated with the indicated unconjugated anti-mouse TREM-1 antibodies at saturating concentrations for 15 mins on ice. Following incubation with these antibodies, cells were then stained with the indicated Alexa Fluor 647 conjugated antibodies for 30 mins on ice. The ability of conjugated antibodies to bind in the presence of unconjugated antibodies was assessed by Alexa 647 signal measured by flow cytometry (BD Fortessa X-14, BD Biosciences, San Jose, CA). Each antibody was validated to block itself for an internal experimental control.

### Source of Antibodies

Pi-9067L, Pi-4928, and Pi-9772 were made by Pionyr Immunotherapeutics. The sequences for Pi-9067L, Pi-4928, and Pi-9772 are shown in Table 2.

### Epitope Mapping

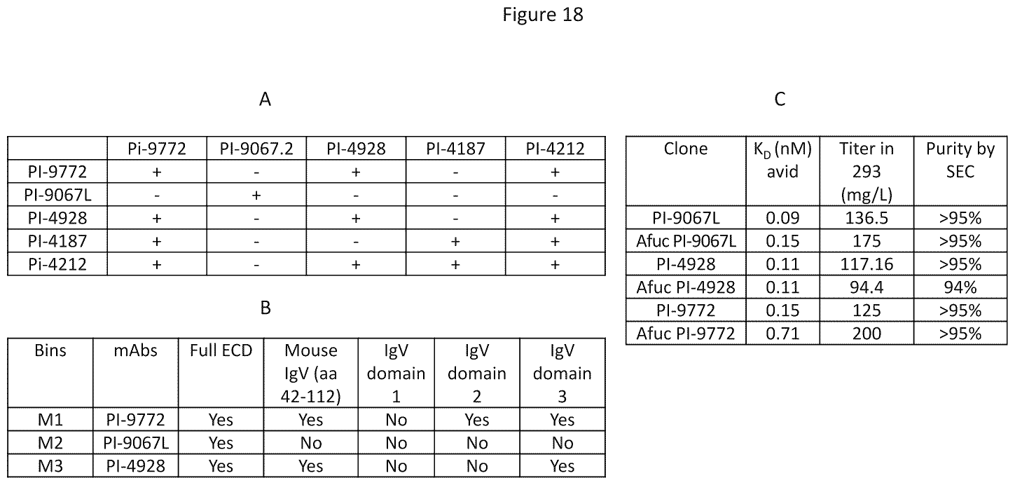

Epitope mapping of the anti-mouse TREM1 mAbs, Pi-9067L, Pi-4928, and Pi-9772 was carried out using mAb cross-blocking experiments by flow cytometry. As shown in FIG. 18A, the three anti-TREM1 mAbs have distinct blocking characteristics when assessed against a range of internal and commercial mAbs, indicating they bind discrete epitopes on mTREM1. The sequences of the mouse IgV domains 1, 2, and 3 are shown in Table 2. The sequences denote the linear amino acid stretches that comprise each domain and the numbering is according to UniProt sequence analysis and 3D modelling of mouse TREM1.

In addition to flow cytometry-based blocking experiments, chimeric constructs of human and mouse TREM1 were made by substituted the full human IgV as well as distinct domains of the IgV into mouse TREM1 and vice versa. This allowed determination of the domains critical for the binding of each mAb to the wild type sequence. As shown in FIG. 18B, this orthogonal approach also demonstrated that Pi-9067L, Pi-4928, and Pi-9772 bind distinct epitopes on mouse TREM1.

### Biochemical Characterization

### Protein Binding (Kd Measurement):

Binding kinetics of the indicated anti-mouse TREM1 antibodies were assessed against a mouse TREM1-hIgG1Fc fusion protein. Plots were generated by applying the 1:1 binding Langmuir model which were overlaid on the binding curves associated differential concentrations of each indicated antibody. Each indicated antibody was injected over captured mouse TREM1-hIgG1Fc fusion protein at a flow rate of 30-50 uL/min for 120-180s and with a dissociation time of 900s in PBS containing Tween-20 using a Biacore T200 instrument.

The biochemical properties of the anti-mouse TREM1 mAbs, PI-9067L, PI-4928, and PI-9772 were determined using several standard biochemical techniques including binding kinetics, production titer, and SEC. FIG. 18C shows a summary of the biochemical characterization of the three mAbs. Based on the biochemical characterization, all three mAbs have suitable properties for in vivo characterization.

### Neutrophil Binding

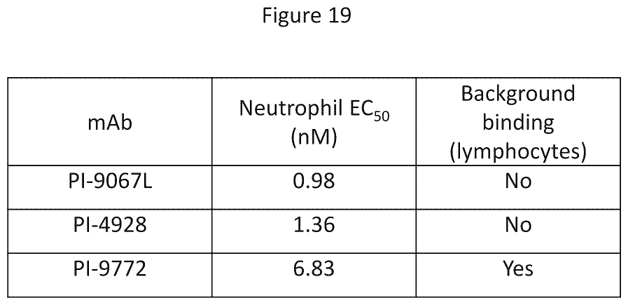

To determine whether the mouse mAbs could specifically bind endogenously expressed mouse TREM1, Pi-9067L, Pi-4928, and Pi-9772 were titrated on BALB/c splenocytes. Spleens were harvested from BALB/c mice and were processed into single cell splenocyte suspensions. The indicated anti-mouse TREM1 antibodies were assessed for their ability to bind splenocyte populations by FACS analysis in a titration-dependent manner. Briefly, splenocytes were labelled with a live/dead viability marker and reagents that block Fc receptors. A backbone cocktail containing antibodies that can subset major myeloid and lymphocyte populations were then used to delineate neutrophils. Specific gating on neutrophils (CD24+Ly6G+Ly6C+CD11b+) and lymphocytes (CD3+NK1.1+B220+CD19+NKp46+) was used to assess specific mouse TREM1 staining by each antibody. FIG. 19 shows that Pi-9067L and Pi-4928 showed specific staining on neutrophils with the strongest EC50 values. These mAbs showed no background binding to lymphocytes. In contrast, Pi-9772 showed background binding to lymphocytes and had the weakest EC50 value on neutrophils. In summary, this data shows that PI-9067L and PI-4928 have excellent binding and biophysical characteristics and recognize endogenous mouse TREM1 with high affinity and specificity. Furthermore, they each bind distinct epitopes on mouse TREM1 and can be used to assess anti-tumor efficacy of anti-TREM1 antibodies in vivo.

### 7.11. Example 11: Binding and ADCC Properties of Pi-9067L, Pi-9772, and Pi-4928 mAbs

### Cellular Binding (EC50 Measurement):

100,000 HEK 293 parental or over-expressing mouse TREM-1 were plated in 96 well plates and dead cells were stained with Zombie Near Infrared (Biolegend, San Diego, CA). Titrations of indicated unconjugated fucosylated or afucosylated antibodies (of Pi-9067L, Pi-9772, and Pi-4928) were incubated with these cells within a range of 0 ug/ml to 30 ug/ml in a 1:4 or 1:5 dilution range across 8 points. These primary unconjugated antibodies were detected with Alexa Fluor 647 conjugated anti-mouse Fcγ secondary antibodies (Jackson Immunoresearch, West Grove, PA). Alexa Fluor 647 signal was measured by flow cytometry (BD Fortessa X-14, BD Biosciences, San Jose, CA). EC50 values were calculated by curve fitting signal generated from antibodies binding to over-expressing cells over background fluorescence generated from HEK293 parental cells in Graphpad Prism (Graphpad Software, La Jolla, CA).

### ADCC Reporter Assay (EC50 Measurement):

Wild type or afucosylated antibodies (Pi-9067L, Pi-9772, and Pi-4928) were assayed for ADCC and activity against cells over expressing mouse TREM-1.

25,000 HEK 293 parental or over-expressing mouse TREM-1 were plated in white flat bottom 96 well plates (Costar). Titrations of indicated unconjugated antibodies were incubated with these cells within a range of 0 ug/ml to 10 ug/ml in a 1:4 or 1:5 dilution range across 8 points for 10 mins at room temperature. After incubation with antibodies, 75,000 Jurkat reporter cells over-expressing mFcγRIV and NFAT responsive luciferase (Promega, Madison, WI) were added to labelled cells at a 3:1 effector to target ratio. The co-culture was incubated for 6 hours at 37° c. in a 5% CO₂ atmosphere. NFAT (luciferase) activity was measured by adding luciferase assay substrate and reagent to all wells (Promega, Madison, WI). Luminescence was read using a Spectramax plate reader (Molecular Devices). EC50 values were calculated by curve fitting luciferase signal generated from antibodies binding to over-expressing cells over background luminescence generated from HEK293 parental cells in Graphpad Prism (Graphpad Software).

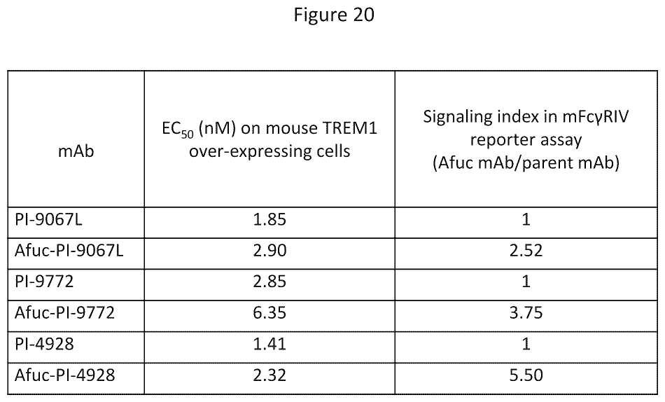

The results for the cellular binding and the ADCC assay are shown in FIG. 20. Pi-9067L, Pi-9772, and Pi-4928 TREM1 mAbs all demonstrated low single digit nanomolar EC50 values when titrated on cells over-expressing mouse TREM1. Afucosylation did not significantly impact the EC50 values of the mAbs, but did enhance their ability to induce signaling through mouse FcγRIV, a surrogate for ADCC. In summary, this data indicates that afucosylation of Pi-9067L, Pi-9772, and Pi-4928 does not impact the ability of these antibodies to bind mouse TREM1, but does significantly enhance their ability to bind FcγR and induce ADCC signaling.

### 7.12. Example 12. Receptor Occupancy (RO) and Pharmacodynamic (PD) Studies

### Receptor Occupancy

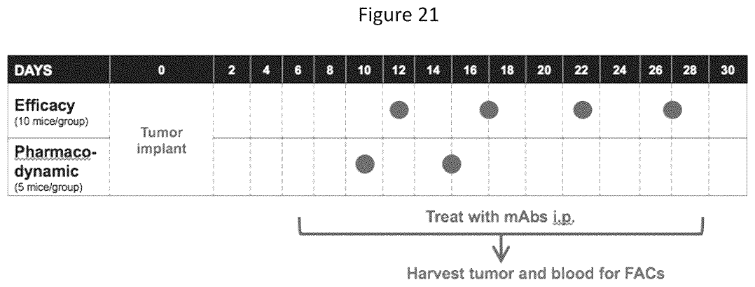

In vivo studies were conducted with Pi-9067L and Pi-4928 in the CT26 and MC38 syngeneic tumor models. An exemplary study design for the mouse tumor studies is shown in FIG. 21.

CT26 (colon adenocarcinoma cells) were harvested using StemPro Accutase Dissociation reagent (Gibco Corporation), washed in PBS and subcutaneously injected into female BALB/c mice (Taconic Biosciences, Hudson, NY) at a dose of 1×10⁶ cells in the right ventral flank. Tumor growth was monitored until the average size of tumors was between 75-130 mm³. MC38 (colon adenocarcinoma cells) were harvested using StemPro Accutase Cell Dissociation reagent (Gibco Corporation, ThermoFisher Scientific, Waltham, MA), washed in PBS and subcutaneously injected into female C57BL/6 mice (Jackson Laboratories, Bar Harbor, ME) at a dose of 8×10⁵ cells in the right ventral flank. Tumor growth was monitored until the average size of tumors was between 75-130 mm³. Mice were subsequently randomized into 3 treatment groups representing an isotype control (mIgG2a), and two anti-mouse TREM1 clones designated Pi-9067L and Pi-4928. One cohort of mice were used for pharmacodynamic and receptor occupancy (5 mice per group) and a second cohort was used for efficacy (10 mice per group) evaluation of Pi9067L and Pi-4928. Antibody treatments were 15 mg/kg. Pharmacodynamic and receptor occupancy studies were performed one to two days after the second mAb dose (mAbs were dosed every 5 days), where tumors and peripheral blood were collected and processed for flow cytometry. Efficacy treatments were also every 5 days and up to 5 doses were given. Tumor size was assessed using the formula V (mm³)=(LxWxW)÷2; where W=width, L=length and V=volume. Tumors were measured twice to three times weekly using calipers and mice were euthanized when tumor volume reached 2000 mm³. Receptor occupancy for Pi-9067L and Pi 4928 in both the CT26 and MC38 models was assessed as previously described in Example 4.

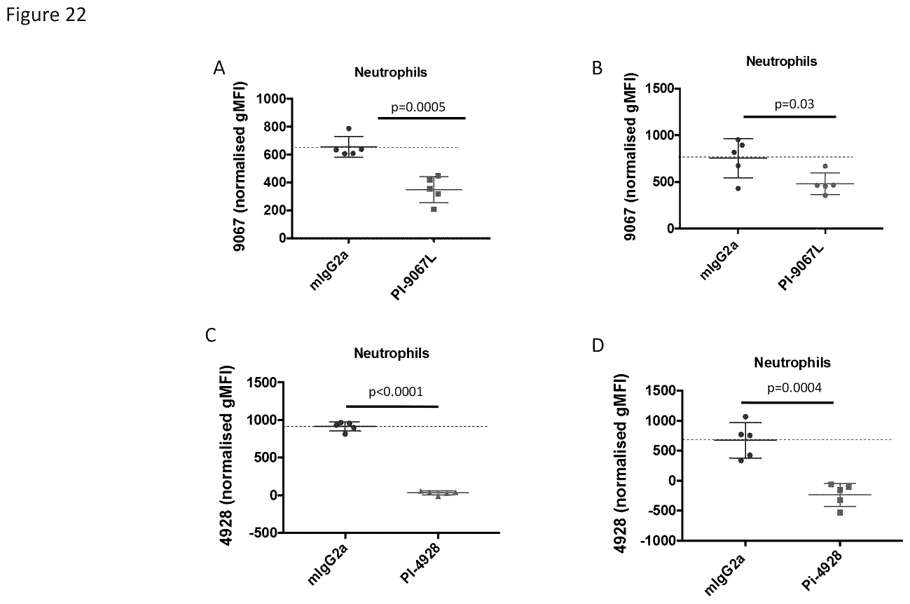

As shown in FIG. 22, RO was observed on intratumoral neutrophils with both PI-9067L and PI-4928 treatment in the CT26 (FIGS. 22A and 22C) and MC38 models (FIGS. 22B and 22D), indicating that both mAbs adequately enter the tumor-microenvironment and bind to TREM1-expressing immune populations. RO was also observed on other intratumor myeloid populations such as TAMs, monocytes, and DC subsets, but not lymphocyte populations (data not shown). This is in contrast to naïve mice where TREM1 expression was only observed on circulating neutrophils in peripheral blood. This indicates that TREM1 is upregulated on a range of myeloid populations within the tumor microenvironment.

### Pharmacodynamics

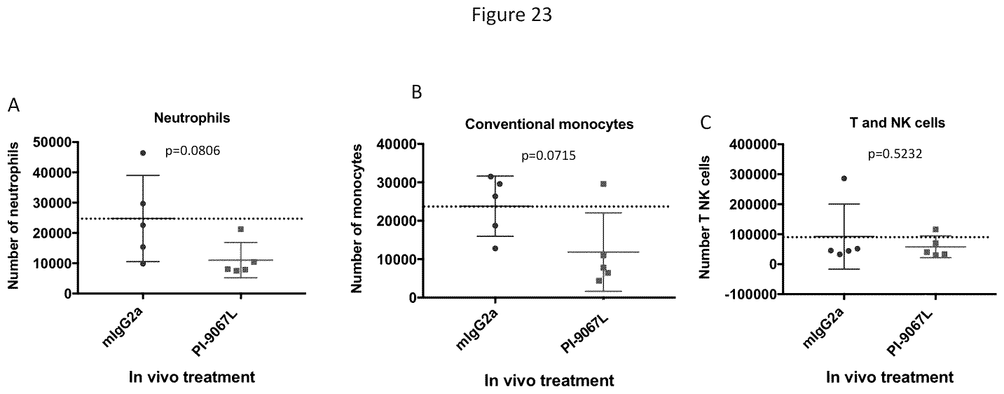

For PD analysis, depletion of TREM1 expressing cells was measured. FIG. 23 shows intratumoral depletion of monocytes and neutrophils by Pi-9067L in the CT26 model. Absolute numbers of monocytes and neutrophils were reduced by 2-fold and 2.3-fold respectively. Consistent with the RO data, T and NK cells were not affected. In contrast to the CT26 data, no significant depletion of TREM1-expressing populations was observed in the MC38 model (data not shown). Collectively, this data demonstrates that Pi-9067L is able to specifically deplete TREM1-expressing cells within the CT26 tumor microenvironment.

### 7.13. Example 13: CT26 In Vivo Combination Therapy Efficacy

Pi-9067L and afucosylated Pi-9067L (Afuc-Pi-9067L) were tested in combination with anti-PD-1 in the CT26 tumor model. CT26 (colon adenocarcinoma cells) were harvested using StemPro Accutase Dissociation reagent (Gibco Corporation), washed in PBS and subcutaneously injected into female BALB/c mice (Taconic Biosciences, Hudson, NY) at a dose of 1×10⁶ cells in the right ventral flank. Tumor growth was monitored until the average size of tumors was between 75-130 mm 3 and mice were subsequently randomized into 6 treatment groups representing isotype treatment (mIgG2a and mIgG1), single agent anti-mouse TREM-1 (Pi-9067L and afucosylated Pi-9067L with isotype control), single agent anti-mouse PD-1 (anti-PD-1 with isotype control), and combination treatment (Pi-9067L and afucosylated Pi-9067L with anti-PD-1). Antibody doses for mIgG2a, Pi-9067L, and Afuc-Pi-9067L were 15 mg/kg and for mIgG1 and anti-PD-1 were 5 mg/kg. Treatments were Day 9, 14, and 19 post tumor inoculation. Tumor size was assessed using the formula V (mm³)=(LxWxW)÷2; where W=width, L=length and V=volume. Tumors were measured two to three times weekly using calipers and mice were euthanized when tumor volume reached 2000 mm³.

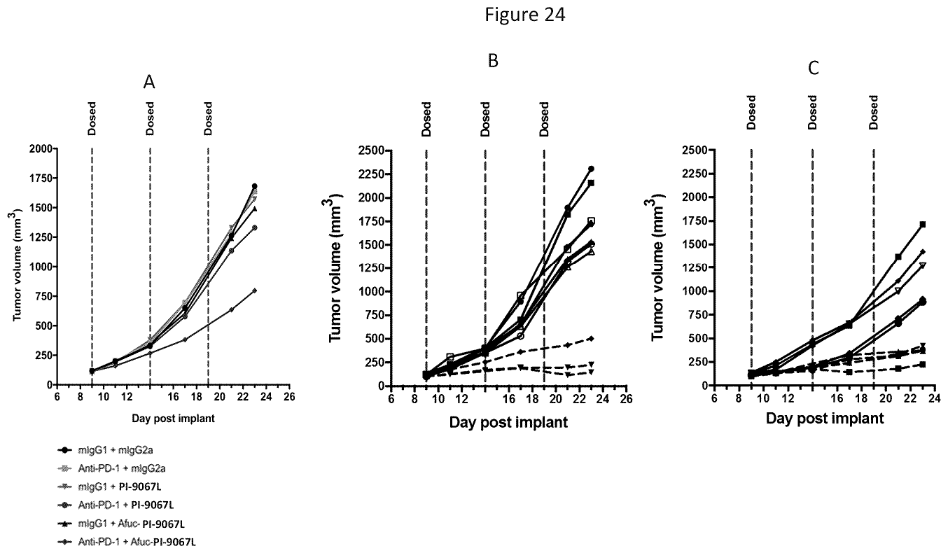

As seen in FIG. 24, Afuc-PI-9067L had better anti-tumor activity when combined with anti-PD-1 than did PI-9067L (FIG. 24A). The impact of afucosylation of PI-9067L on anti-tumor activity was more clearly seen in the analysis of the individual mouse tumor volumes. FIG. 24B shows the individual tumor volumes for Pi-9067L in combination with anti-PD-1. FIG. 24C shows the individual tumor volumes for Afuc-Pi-9067L in combination with anti-PD-1. Compared to the Pi-9067L combination cohort, there were more mice that were classed as responders in the Afuc-PI-9067L combination cohort ( 3/10 compared to 5/10 respectively), and the mean overall tumor volume for each mouse was lower (Afuc-Pi-9067=796.8±525.1 mm3 and Pi-9067=1330±772.8 mm3). This data indicates that afucosylation of PI-9067L gives better anti-tumor efficacy in the CT26 model than fucosylated PI-9067L, likely due to the enhanced ability of the afucosylated version to bind to FcγRs.

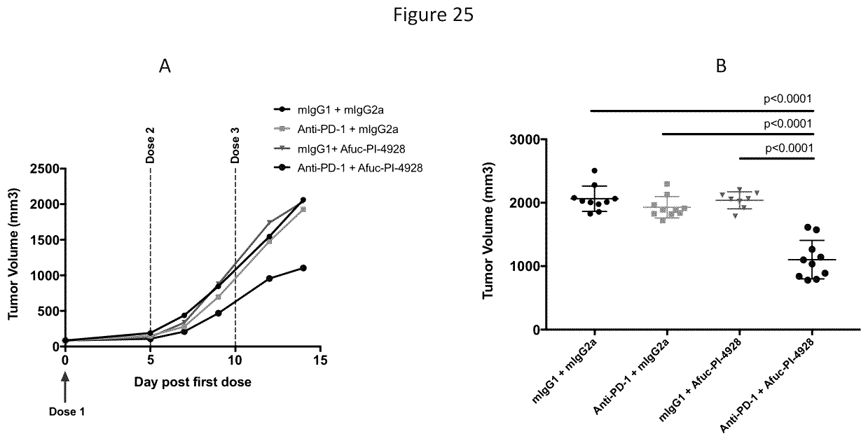

Since Afuc-PI-9067L induced more robust anti-tumor efficacy in the CT26 model than WT PI-9067L in combination with anti-PD-1, the therapeutic efficacy of Afuc-PI-4928 in combination with anti-PD-1 was also assessed in this model. As shown in FIG. 25A, compared to the control groups, the combination of Afuc-Pi-4928 and anti-PD-1 also showed combinatorial therapeutic efficacy. When endpoint tumor volumes were assessed, the mean tumor sizes in the Afuc-Pi-4928+anti-PD-1 combination were significantly different to all the control groups (FIG. 25B).

### 7.14. Example 14: MC38 In Vivo Efficacy with Pi-9067 and Afucosylated Pi-9067

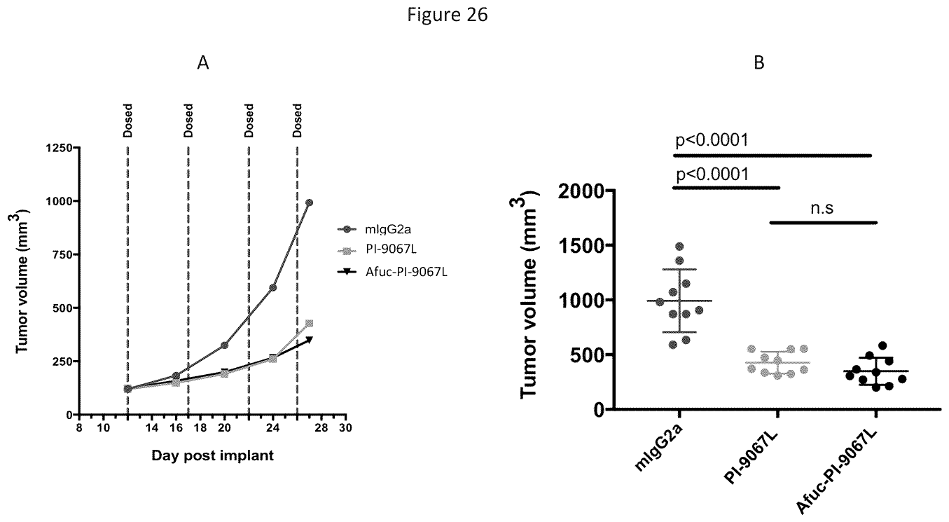

Pi-9067L was also observed to elicit significant anti-tumor activity as a monotherapy in the MC38 colon tumor model as shown in FIGS. 26A and 26B. MC38 (colon adenocarcinoma cells) were harvested using StemPro Accutase Cell Dissociation reagent (Gibco Corporation, ThermoFisher Scientific, Waltham, MA), washed in PBS and subcutaneously injected into female C57BL/6 mice (Jackson Laboratories, Bar Harbor, ME) at a dose of 8×10⁵ cells in the right ventral flank. Tumor growth was monitored until the average size of tumors was between 75-130 mm³ and mice were subsequently randomized into 3 treatment groups representing an isotype control (mIgG2a), and fucosylated and afucosylated anti-mouse TREM1 Pi-9067L. Antibody treatments were all 15 mg/kg and treatments were Day 12, 17, 22, and 26 post tumor inoculation. Tumor size was assessed using the formula V (mm³)=(LxWxW)÷2; where W=width, L=length and V=volume. Tumors were measured two to three times weekly using calipers and mice were euthanized when tumor volume reached 2000 mm³.

As shown in FIG. 26, Afuc-Pi-9067L did not show an increase in therapeutic benefit compared to WT Pi-9067L. Since there was no observed depletion of TREMV cells in the MC38 model upon treatment with PI-9067L, the lack of further therapeutic benefit with afucosylation is consistent with this observation.

### 7.15. Example 15: Py8119 In Vivo Combination Therapy Efficacy

The therapeutic efficacy of Afuc-PI-4928 in combination with anti-PD-1 in the Py8119 model of triple negative breast cancer was also assessed.

The Py8119 breast carcinoma line (ATCC, Manassas, VA) was harvested in log phase and injected subcutaneously into the right ventral flank of female C57BL/6 mice (Jackson Laboratories) at a dose of 2×10⁶ cells/mouse mixed with an equal volume of Matrigel. After tumors in the majority of mice had reached 70-130 mm³, mice were randomized and dosing with the indicated antibodies was commenced. Antibodies were dosed intraperitoneally at 15 mg/kg for Afuc-PI-4928 and a mIgG2a isotype, and 5 mg/kg for anti-PD1 and a mIgG1 isotype at a frequency of every 5 days up to 4 doses. Tumor growth was measured over time by caliper measurements using the formula, Volume (mm3)=[Length (mm)×Width (mm)×Width (mm)]/2.

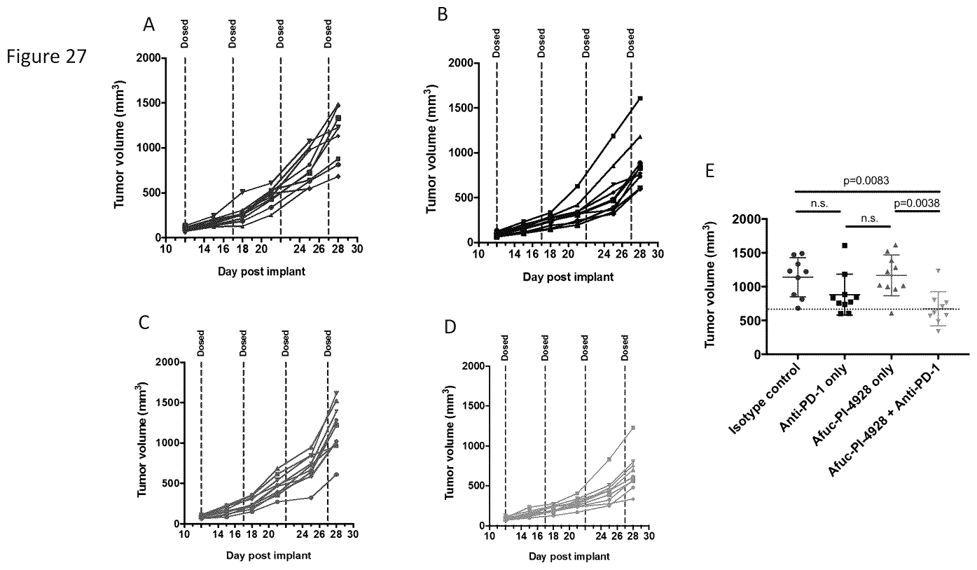

Individual tumor curves from each group are shown in FIG. 27. FIG. 27A shows Isotype control, FIG. 27B shows anti-PD-1 treatment, FIG. 27C shows Afuc-Pi-4928 treatment, and FIG. 27D shows Afuc-Pi-4928+anti PD-1 treatment. Endpoint tumor volumes for each treatment are shown in FIG. 27E. The endpoint tumor volumes in the combination arm were significantly lower than the isotype control and Afuc-PI-4928 control cohorts. In contrast, the anti-PD-1 only control cohort was not significantly different to any other group. Thus, the combination of Afuc-PI-4928 with anti-PD-1 demonstrated stronger anti-tumor activity in the Py8119 model compared to anti-PD-1 or Afuc-PI-4928 alone.

### 7.16. Example 16: Pharmacokinetics (PK) of Pi-9067L and Pi-4928

CT26 or MC38 tumor bearing mice were dosed twice with Pi-9067L, Pi-4928, or their afucosylated counterparts. Pi-9067L and Pi-4928 mAb levels in the plasma of CT26 or MC38 tumor-bearing mice were assessed 48 hrs after the second dose of mAb. Serum levels of anti-mouse TREM1 mAbs were determined using a standard ligand-binding ELISA format. Mouse TREM1 His-tagged fusion construct was coated onto Nunc MaxiSorp flat-bottom plates followed by blocking unbound sites with BSA. Pi-9067L and Pi-4928 (fucosylated or afucosylated) mAb standards or mouse plasma samples were diluted in assay buffer containing Tween detergent and added to antigen-coated plates. The bound mAbs were detected with anti-mouse IgG2a secondary mAb conjugated to HRP for colorimetric quantitation by optical density absorbance.

### In Vivo Plasma Concentrations

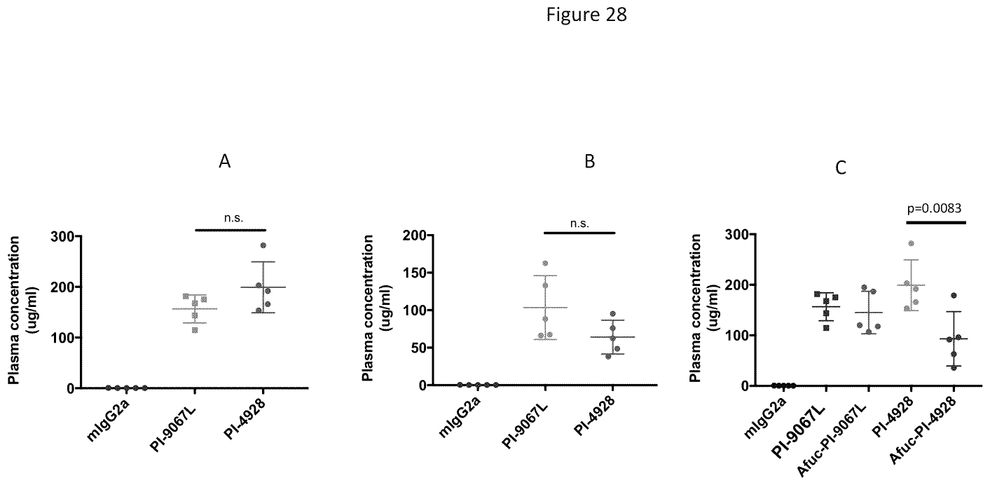

FIG. 28A shows the mean plasma levels of Pi-9067L and Pi-4928 in CT26 tumor-bearing mice. There was no significant difference in the plasma concentrations of Pi-9067L and Pi-4928. Similar results were seen in the MC38 model (FIG. 28B). Next, PK properties of afucosylated mAbs were also assessed in comparison to the fucosylated antibody counterparts. As seen in FIG. 28C, in CT26 tumor-bearing mice, there was no significant difference in the plasma concentration between parent Pi-9067L and Afuc-Pi-9067L. However, there was a significant difference between the plasma concentrations of the parent Pi-4928 and Afuc-Pi-4928. Although there was a difference in the plasma concentration of Pi-4928 and Afuc-Pi-4928, therapeutic efficacy of Afuc-Pi-4928 with anti-PD-1 was observed in the both the CT26 and Py8119 models (Examples 13 and 15). Furthermore, full receptor occupancy of TREM1 was observed in receptor occupancy studies with mice were dosed with Afuc-PI-4928 (data not shown). This indicates despite the lower plasma concentration of Afuc-Pi-4928 compared to wild-type Pi-4928, there is still enough circulating antibody to bind to TREM1 in the TME and induce therapeutic efficacy.

### Toxicology

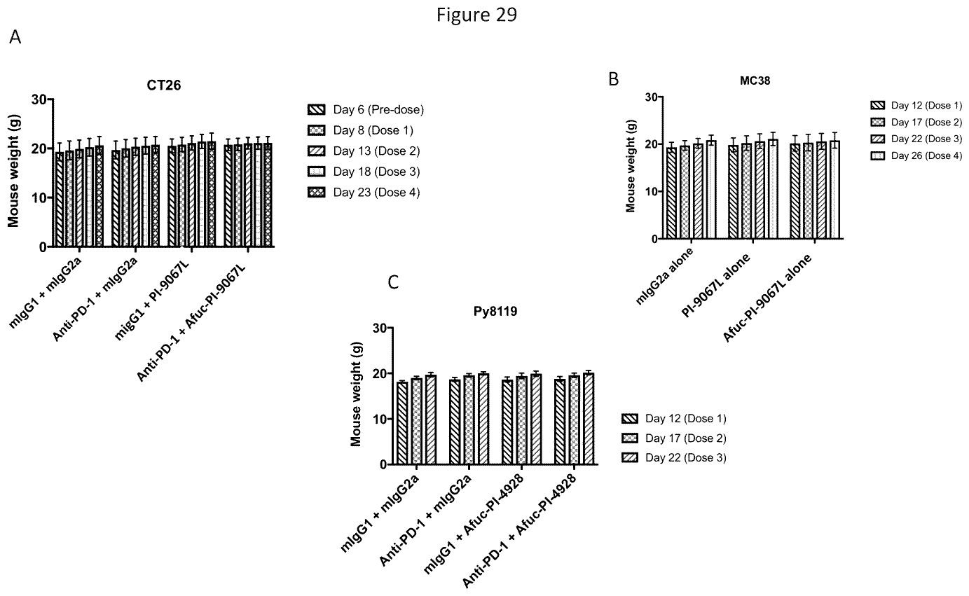

Mice were dosed 4 times for up to 26 days with PI-9067L alone, Afuc-PI-9067L alone, or in combination with anti-PD-1 and weight loss monitored for 26 days. The treatment did not result in any weight loss in the anti-TREM1 antibody treated mice (FIGS. 29A and 29B) compared to control treated mice, or within the same group over time in the CT26 and MC38 models. Similarly, mice dosed 3 times for 22 days with Afuc-PI-4928 alone or in combination with anti-PD-1 did not show weight loss in the Py8119 model (FIG. 29C). None of the mice in any of the treatment groups across experiments displayed any observable abnormal behavior or appearance.

### Peripheral Neutropenia

Since TREM1 is expressed on neutrophils in the tumor microenvironment as well as the periphery, peripheral neutropenia induced by Pi-9067L or Pi-4928 was assessed.

Naïve BALB/c (Taconic) or C57BL/6 mice (Jackson Laboratories) were treated with 15 mg/kg of mIgG2a or PI-9067L at Day 0 and Day 5. At Day 7 (48 hrs after the second dose), blood was collected and the percentage of neutrophils was assessed by specific gating on Ly6G and CD11b double positive events by flow cytometry.

The CT26 colon carcinoma line (ATCC, Manassas, VA) was harvested in log phase and injected subcutaneously into the right ventral flank of female BALB/c mice (Taconic) at a dose of 1×10⁶ cells/mouse. After tumors in the majority of mice had reached 75-135 mm³, mice were randomized and dosing with the indicated antibodies (afucosylated or wild-type Pi-9067L or Pi-4928 or isotype control) was commenced. Antibodies were dosed intraperitoneally at 15 mg/kg every 5 days, and blood was collected 48 hrs after the second dose. The percentage of neutrophils was assessed by specific gating on Ly6G and CD11b double positive events by flow cytometry.

The MC38 colon carcinoma line (ATCC, Manassas, VA) was harvested in log phase and injected subcutaneously into the right ventral flank of female C57BL/6 mice (Jackson Laboratories) at a dose of 8×10⁵ cells/mouse. After tumors in the majority of mice had reached 75-135 mm³, mice were randomized and dosing with the indicated antibodies (afucosylated or wild-type Pi-9067L or Pi-4928 or isotype control) was commenced. Antibodies were dosed intraperitoneally at 15 mg/kg every 5 days, and blood was collected 48 hrs after the second dose. The percentage of neutrophils was assessed by specific gating on Ly6G and CD11b double positive events by flow cytometry.

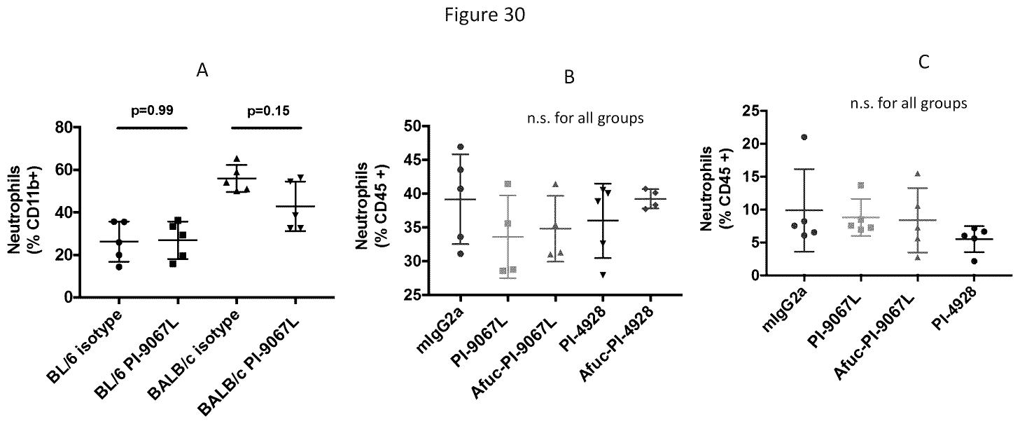

Pi-9067L did not induce peripheral neutropenia in non-tumor bearing C57BL/6 and BALB/c mice (FIG. 30A). PI-9067L and PI-4928 as well as their afucosylated variants also did not yield peripheral neutropenia in CT26 tumor bearing mice (FIG. 30B) or MC38 tumor bearing mice (FIG. 30C). n.s. denotes not significant. In summary, this data shows that fucosylated and afucosylated PI-9067L and PI-4928 have excellent pre-clinical safety characteristics. Furthermore, fucosylated and afucosylated PI-9067L and PI-4928 display good pharmacokinetic properties since circulating antibody in the plasma can be detected at least 48 hrs after the second dose of antibody while simultaneously achieving full intratumoral RO.

### 7.17. Example 17: CDC Activity of Anti-mTREM1 and Anti-hTREM1 Antibodies

Target cells expressing human TREM1 are harvested from culture in log phase and 5×10⁴-2×10⁵ cells are plated in 96 well U bottom plates. After plating, a 1:1 mixture of human complement containing serum and anti-human and anti-mouse TREM1 mAbs (hIgG1 isotype) are added, and incubated with the target cells for 2-3 hrs at 37° C. After incubation, cells are assessed for antibody-mediated CDC activity by assessing cell death by flow cytometry or luminescence-based methods such as Cell Titer Glo (Promega, Madison, WI).

TABLE 2
SEQUENCES

| SEQ ID | | |
|---|---|---|
| NO | ID | Sequence |
| 1 | Pi-9067 | EVQLVESGGGLVQPGGSLRLSCAASGFDIYSYSIHWVRQAPGKGLEWVAYTYPSYGYTSYA |
| | VH | DSVKGRFTISADTSKNTAYLQMNSLRAEDTAVYYCARGAVAALDYWGQGTLVTVSS |
| 2 | Pi-9067 | DIQMTQSPSSLSASVGDRVTITCRASQSVSSAVAWYQQKPGKAPKLLIYSASSLYSGVPSR |
| | VL | FSGSRSGTDFTLTISSLQPEDFATYYCQQYGVSGYSLITFGQGTKVEIK |
| 3 | Pi-9068 | EVQLVESGGGLVQPGGSLRLSCAASGFDISSSSMHWVRQAPGKGLEWVASISSSYSYTYYA |
| | VH | DSVKGRFTISADTSKNTAYLQMNSLRAEDTAVYYCARYVAMDYWGQGTLVTVSS |
| 4 | Pi-9068 | DIQMTQSPSSLSASVGDRVTITCRASQSVSSAVAWYQQKPGKAPKLLIYSASSLYSGVPSR |
| | VL | FSGSRSGTDFTLTISSLQPEDFATYYCQQWGGGHYLFTFGQGTKVEIK |
| 5 | Pi-9069 | EVQLVESGGGLVQPGGSLRLSCAASGFDLYSSYIHWVRQAPGKGLEWVASTYPSYGYTYYA |
| | VH | DSVKGRFTISADTSKNTAYLQMNSLRAEDTAVYYCARSHSYVHGAMDYWGQGTLVTVSS |
| 6 | Pi-9069 | DIQMTQSPSSLSASVGDRVTITCRASQSVSSAVAWYQQKPGKAPKLLIYSASSLYSGVPSR |
| | VL | FSGSRSGTDFTLTISSLQPEDFATYYCQQYWWVPAAPITFGQGTKVEIK |
| 7 | 9067 CDR- | IYSYSI |
| | H1 | |
| 8 | 9067 CDR- | YIYPSYGYTS |
| | H2 | |
| 9 | 9067 CDR- | GAVAAL |
| | H3 | |
| 10 | 9067 CDR- | SVSSA |
| | L1 | |
| 11 | 9067 CDR- | SASSLYS |
| | L2 | |
| 12 | 9067 CDR- | YGVSGYSLI |
| | L3 | |
| 13 | 9068 CDR- | ISSSSM |
| | H1 | |
| 14 | 9068 CDR- | SISSSYSYTY |
| | H2 | |
| 15 | 9068 CDR- | YVAM |
| | H3 | |
| 16 | 9068 CDR- | SVSSA |
| | L1 | |
| 17 | 9068 CDR- | SASSLYS |
| | L2 | |
| 18 | 9068 CDR- | WGGGHYLF |
| | L3 | |
| 19 | 9069 CDR- | LYSSYI |
| | H1 | |
| 20 | 9069 CDR- | SIYPSYGYTY |
| | H2 | |
| 21 | 9069 CDR- | SHSYVHGAM |
| | H3 | |
| 22 | 9069 CDR- | SVSSA |
| | L1 | |
| 23 | 9069 CDR- | SASSLYS |
| | L2 | |
| 24 | 9069 CDR- | YWWVPAAPI |
| | L3 | |
| 25 | mAb0170 | EVQLVESGGGLVQPGGSLKLSCAASGFTFSTYAMHWVRQASGKGLEWVGRIRTKSSNYATY |
| | VH | YAASVKGRFTISRDDSKNTAYLQMNSLKTEDTAVYYCTRDMGIRRQFAYWGQGTLVTVSS |
| 26 | mAb0170 | DIVLTQSPDSLAVSLGERATINCRASESVDTFDYSFLHWYQQKPGQPPKLLIYRASNLESG |
| | VL | VPDRFSGSGSGTDFTLTISSLQAEDVAVYYCQQSNEDPYTFGQGTKLEIK |
| 27 | Pi-8421 | EVQLVESGGGLVQPGGSLRLSCAASGFDISYSSIHWVRQAPGKGLEWVASIYSSYGSTYYA |
| | VH | DSVKGRFTISADTSKNTAYLQMNSLRAEDTAVYYCARYYYSHAGWYVSGYWPAIDYWGQGT |
| | | LVTVSS |
| 28 | Pi-8421 | DIQMTQSPSSLSASVGDRVTITCRASQSVSSAVAWYQQKPGKAPKLLIYSASSLYSGVPSR |
| | VL | FSGSRSGTDFTLTISSLQPEDFATYYCQQSSYSLITFGQGTKVEIK |
| 29 | Pi-8419 | EVQLVESGGGLVQPGGSLRLSCAASGFDLYSSSIHWVRQAPGKGLEWVASISPYYSSTSYA |
| | VH | DSVKGRFTISADTSKNTAYLQMNSLRAEDTAVYYCARSVYYTVYFGLDYWGQGTLVTVSS |
| 30 | Pi-8419 | DIQMTQSPSSLSASVGDRVTITCRASQSVSSAVAWYQQKPGKAPKLLIYSASSLYSGVPSR |
| | VL | FSGSRSGTDFTLTISSLQPEDFATYYCQQAVGAWGYLITFGQGTKVEIK |
| 31 | mAb0170 | TYAMH |
| | CDR-H1 | |
| 32 | mAb0170 | RIRTKSSNYATYYAASVKG |
| | CDR-H2 | |
| 33 | mAb0170 | DMGIRRQFAY |
| | CDR-H3 | |
| 34 | mAb0170 | RASESVDTFDYSFLH |
| | CDR-L1 | |
| 35 | mAb0170 | RASNLES |
| | CDR-L2 | |
| 36 | mAb0170 | QQSNEDPYT |
| | CDR-L3 | |
| 37 | Pi-8421 | ISYSSI |
| | CDR-H1 | |
| 38 | Pi-8421 | SIYSSYGSTY |
| | CDR-H2 | |
| 39 | Pi-8421 | YYYSHAGWYVSGYWPAI |
| | CDR-H3 | |
| 40 | Pi-8421 | SVSSA |
| | CDR-L1 | |
| 41 | Pi-8421 | SASSLYS |
| | CDR-L2 | |
| 42 | Pi-8421 | SSYSLI |
| | CDR-L3 | |
| 43 | Pi-8419 | LYSSSI |
| | CDR-H1 | |
| 44 | Pi-8419 | SISPYYSSTS |
| | CDR-H2 | |
| 45 | Pi-8419 | SVYYTVYFGL |
| | CDR-H3 | |
| 46 | Pi-8419 | SVSSA |
| | CDR-L1 | |
| 47 | Pi-8419 | SASSLYS |
| | CDR-L2 | |
| 48 | Pi-8419 | AVGAWGYLI |
| | CDR-L3 | |
| 49 | Pi-9772 | EVQLVESGGGLVQPGGSLRLSCAASGFDLSYSSMHWVRQAPGKGLEWVAYISPYSGYTSYA |
| | VH | DSVKGRFTISADTSKNTAYLQMNSLRAEDTAVYYCARHGSWSYGFSSGFDYWGQGTLVTVS |
| | | S |
| 50 | Pi-9772 | DIQMTQSPSSLSASVGDRVTITCRASQSVSSAVAWYQQKPGKAPKLLIYSASSLYSGVPSR |
| | VL | FSGSRSGTDFTLTISSLQPEDFATYYCQQYAYYYPITFGQGTKVEIK |
| 51 | Pi-4928 | QVQLKESGPGLVQPSQTLSLTCTVSGFSITSSNVYWFRQPPGKGLEWMGNIWGDGSTDYNS |
| | VH | SLKSRLTLSRDTSKNQVFLKINSLQSEDTATYFCTRSWEYYFDHWGQGVVVTVSS |
| 52 | Pi-4928 | DIVLTQSPALAVSPGQRATLSCRASQSVTLSNVNLMNWYQQKPGQQPKLLIYHASNLASGI |
| | VL | PTRFSGSGSGTDFTLTIDPVQADDIAAYYCQQSGESPRTFGGGTKVELK |
| 53 | Pi-4212 | QVQLQQSGAELVKPGSSVKLSCKASGYTFTSYDMHWIKQQPGDGLEWLGWLYPGNGNSKY |
| | VH | NQKFDGKATLTADKSSSTAYLQLSSLTSEDSAVYFCARRGEFTYYFDYWGQGVMVTVSS |
| 54 | Pi-4212 | DIQMTQSPASLSASLGETVTIECLASEDIYSNLAWYQQKPGKSPQLLLYYANSLNDGVPSR |
| | light | FSVSGSGTQYSLKINSLQSEDVSLYFCQQHYDSPYTFGAGTKLELK |
| | chain | |
| 55 | Pi-9772 | LSYSSM |
| | CDR-H1 | |
| 56 | Pi-9772 | YISPYSGYTS |
| | CDR-H2 | |
| 57 | Pi-9772 | HGSWSYGFSSGF |
| | CDR-H3 | |
| 58 | Pi-9772 | SVSSA |
| | CDR-L1 | |
| 59 | Pi-9772 | SASSLYS |
| | CDR-L2 | |
| 60 | Pi-9772 | YAYYYPI |
| | CDR-L3 | |
| 61 | Pi-4928 | GFSITSSNVY |
| | CDR-H1 | |
| 62 | Pi-4928 | NIWGDGSTDYNSSLKS |
| | CDR-H2 | |
| 63 | Pi-4928 | SWEYYFDH |
| | CDR-H3 | |
| 64 | Pi-4928 | RASQSVTLSNVNLMN |
| | CDR-L1 | |
| 65 | Pi-4928 | HASNLAS |
| | CDR-L2 | |
| 66 | Pi-4928 | QQSGESPRT |
| | CDR-L3 | |
| 67 | Pi-4212 | GYTFTSYDMH |
| | CDR-H1 | |
| 68 | Pi-4212 | WLYPGNGNSKYNQKFDG |
| | CDR-H2 | |
| 69 | Pi-4212 | RGEFTYYFDY |
| | CDR-H3 | |
| 70 | Pi-4212 | LASEDIYSNLA |
| | CDR-L1 | |
| 71 | Pi-4212 | YANSLND |
| | CDR-L2 | |
| 72 | Pi-4212 | QQHYDSPYT |
| | CDR-L3 | |
| 74 | Pi-9067s | EVQLVESGGGLVQPGGSLRLSCAASGFDIYSYSIHWVRQAPGKGLEWVAYTYPSYGYTSYA |
| | heavy | DSVKGRFTISADTSKNTAYLQMNSLRAEDTAVYYCARGAVAALDYWGQGTLVTVSSASTKG |
| | chain | PSVFPLAPSSKSTSGGTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPCPAPNLLGGPSVFIFP |
| | | PKIKDVLMISLSPIVTCVVVDVSEDDPDVQISWFVNNVEVHTAQTQTHREDYNSTLRVVSA |
| | | LPIQHQDWMSGKEFKCKVNNKDLPAPIERTISKPKGSVRAPQVYVLPPPEEEMTKKQVTLT |
| | | CMVTDFMPEDIYVEWTNNGKTELNYKNTEPVLDSDGSYFMYSKLRVEKKNWVERNSYSCSV |
| | | VHEGLHNHHTTKSFSRTPGK |
| 75 | Pi-9067s | DIQMTQSPSSLSASVGDRVTITCRASQSVSSAVAWYQQKPGKAPKLLIYSASSLYSGVPSR |
| | light | FSGSRSGTDFTLTISSLQPEDFATYYCQQYGVSGYSLITFGQGTKVEIKRTVAAPSVFIFP |
| | chain | PSDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 76 | Pi-9067L | EVQLVESGGGLVQPGGSLRLSCAASGFDIYSYSIHWVRQAPGKGLEWVAYTYPSYGYTSYA |
| | heavy | DSVKGRFTISADTSKNTAYLQMNSLRAEDTAVYYCARGAVAALDYWGQGTLVTVSSAKTTA |
| | chain | PSVYPLAPVCGDTTGSSVTLGCLVKGYFPEPVTLTWNSGSLSSGVHTFPAVLQSDLYTLSS |
| | | SVTVTSSTWPSQSITCNVAHPASSTKVDKKIEPRGPTIKPCPPCKCPAPNLLGGPSVFIFP |
| | | PKIKDVLMISLSPIVTCVVVDVSEDDPDVQISWFVNNVEVHTAQTQTHREDYNSTLRVVSA |
| | | LPIQHQDWMSGKEEKCKVNNKDLPAPIERTISKPKGSVRAPQVYVLPPPEEEMTKKQVTLT |
| | | CMVTDFMPEDIYVEWTNNGKTELNYKNTEPVLDSDGSYFMYSKLRVEKKNWVERNSYSCSV |
| | | VHEGLHNHHTTKSFSRTPGK |
| 77 | Pi-9067L | DIQMTQSPSSLSASVGDRVTITCRASQSVSSAVAWYQQKPGKAPKLLIYSASSLYSGVPSR |
| | light | FSGSRSGTDFTLTISSLQPEDFATYYCQQYGVSGYSLITFGQGTKVEIKRADAAPTVSIFP |
| | chain | PSSEQLTSGGASVVCFLNNFYPKDINVKWKIDGSERQNGVLNSWTDQDSKDSTYSMSSTLT |
| | | LTKDEYERHNSYTCEATHKTSTSPIVKSFNRNEC |
| 78 | Pi-9069s | DIQMTQSPSSLSASVGDRVTITCRASQSVSSAVAWYQQKPGKAPKLLIYSASSLYSGVPSR |
| | heavy | FSGSRSGTDFTLTISSLQPEDFATYYCQQYWWVPAAPITEGQGTKVEIKRTVAAPSVFIFP |
| | chain | PSDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 79 | Pi-9069s | DIQMTQSPSSLSASVGDRVTITCRASQSVSSAVAWYQQKPGKAPKLLIYSASSLYSGVPSR |
| | light | FSGSRSGTDFTLTISSLQPEDFATYYCQQYWWVPAAPITFGQGTKVEIKRTVAAPSVFIFP |
| | chain | PSDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 80 | Pi-9069L | EVQLVESGGGLVQPGGSLRLSCAASGFDLYSSYIHWVRQAPGKGLEWVASTYPSYGYTYYA |
| | heavy | DSVKGRFTISADTSKNTAYLQMNSLRAEDTAVYYCARSHSYVHGAMDYWGQGTLVTVSSAK |
| | chain | TTAPSVYPLAPVCGDTTGSSVTLGCLVKGYFPEPVTLTWNSGSLSSGVHTFPAVLQSDLYT |
| | | LSSSVTVTSSTWPSQSITCNVAHPASSTKVDKKIEPRGPTIKPCPPCKCPAPNLLGGPSVF |
| | | IFPPKIKDVLMISLSPIVTCVVVDVSEDDPDVQISWFVNNVEVHTAQTQTHREDYNSTLRV |
| | | VSALPIQHQDWMSGKEEKCKVNNKDLPAPIERTISKPKGSVRAPQVYVLPPPEEEMTKKQV |
| | | TLTCMVTDFMPEDIYVEWTNNGKTELNYKNTEPVLDSDGSYFMYSKLRVEKKNWVERNSYS |
| | | CSVVHEGLHNHHTTKSFSRTPGK |
| 81 | Pi-9069L | DIQMTQSPSSLSASVGDRVTITCRASQSVSSAVAWYQQKPGKAPKLLIYSASSLYSGVPSR |
| | light | FSGSRSGTDFTLTISSLQPEDFATYYCQQYWWVPAAPITFGQGTKVEIKRADAAPTVSIFP |
| | chain | PSSEQLTSGGASVVCFLNNFYPKDINVKWKIDGSERQNGVLNSWTDQDSKDSTYSMSSTLT |
| | | LTKDEYERHNSYTCEATHKTSTSPIVKSFNRNEC |
| 82 | Pi-9772 | EVQLVESGGGLVQPGGSLRLSCAASGFDLSYSSMHWVRQAPGKGLEWVAYISPYSGYTSYA |
| | heavy | DSVKGRFTISADTSKNTAYLQMNSLRAEDTAVYYCARHGSWSYGFSSGFDYWGQGTLVTVS |
| | chain | SAKTTAPSVYPLAPVCGDTTGSSVTLGCLVKGYFPEPVTLTWNSGSLSSGVHTFPAVLQSD |
| | | LYTLSSSVTVTSSTWPSQSITCNVAHPASSTKVDKKIEPRGPTIKPCPPCKCPAPNLLGGP |
| | | SVFIFPPKIKDVLMISLSPIVTCVVVDVSEDDPDVQISWFVNNVEVHTAQTQTHREDYNST |
| | | LRVVSALPIQHQDWMSGKEEKCKVNNKDLPAPIERTISKPKGSVRAPQVYVLPPPEEEMTK |
| | | KQVTLTCMVTDFMPEDIYVEWTNNGKTELNYKNTEPVLDSDGSYFMYSKLRVEKKNWVERN |
| | | SYSCSVVHEGLHNHHTTKSFSRTPGK |
| 83 | Pi-9772 | DIQMTQSPSSLSASVGDRVTITCRASQSVSSAVAWYQQKPGKAPKLLIYSASSLYSGVPSR |
| | light | FSGSRSGTDFTLTISSLQPEDFATYYCQQYAYYYPITFGQGTKVEIKRADAAPTVSIFPPS |
| | chain | SEQLTSGGASVVCFLNNFYPKDINVKWKIDGSERQNGVLNSWTDQDSKDSTYSMSSTLTLT |
| | | KDEYERHNSYTCEATHKTSTSPIVKSFNRNEC |
| 84 | Pi-4928 | QVQLKESGPGLVQPSQTLSLTCTVSGFSITSSNVYWFRQPPGKGLEWMGNIWGDGSTDYNS |
| | heavy | SLKSRLTLSRDTSKNQVFLKINSLQSEDTATYFCTRSWEYYFDHWGQGVVVTVSSAKTTAP |
| | chain | SVYPLAPVCGDTTGSSVTLGCLVKGYFPEPVTLTWNSGSLSSGVHTFPAVLQSDLYTLSSS |
| | | VTVTSSTWPSQSITCNVAHPASSTKVDKKIEPRGPTIKPCPPCKCPAPNLLGGPSVFIFPP |
| | | KIKDVLMISLSPIVTCVVVDVSEDDPDVQISWFVNNVEVHTAQTQTHREDYNSTLRVVSAL |
| | | PIQHQDWMSGKEEKCKVNNKDLPAPIERTISKPKGSVRAPQVYVLPPPEEEMTKKQVTLTC |
| | | MVTDFMPEDIYVEWTNNGKTELNYKNTEPVLDSDGSYFMYSKLRVEKKNWVERNSYSCSVV |
| | | HEGLHNHHTTKSFSRTPGK |
| 85 | Pi-4928 | DIVLTQSPALAVSPGQRATLSCRASQSVTLSNVNLMNWYQQKPGQQPKLLIYHASNLASGI |
| | light | PTRFSGSGSGTDFTLTIDPVQADDIAAYYCQQSGESPRTFGGGTKVELKRADAAPTVSIFP |
| | chain | PSSEQLTSGGASVVCFLNNFYPKDINVKWKIDGSERQNGVLNSWTDQDSKDSTYSMSSTLT |
| | | LTKDEYERHNSYTCEATHKTSTSPIVKSFNRNEC |
| 86 | Pi-4212 | QVQLQQSGAELVKPGSSVKLSCKASGYTFTSYDMHWIKQQPGDGLEWLGWLYPGNGNSKYN |
| | heavy | QKFDGKATLTADKSSSTAYLQLSSLTSEDSAVYFCARRGEFTYYFDYWGQGVMVTVSSTET |
| | chain | TAPSVYPLAPGTALKSNSMVTLGCLVKGYFPEPVTVTWNSGALSSGVHTFPAVLQSGLYTL |
| | | TSSVTVPSSTWSSQAVTCNVAHPASSTKVDKKIVPRECNPCGCTGSEVSSVFLFPPKTKDV |
| | | LTLTLTPKVTCVVVDISQNDPEVRFSWFLDDVEVHTAQTHAPEKQSNSTLRSVSELPIVHR |
| | | DWLDGKTFKCKVNSGAFPAPIEKSLSKPEGTPRGPQVYTMAPPKEEMTQSQVSITCMVKGF |
| | | YPPDLYTEWKMNGQPQENYKNTPPTMDTDGSYFLYSKLNVKKETWQQGNTFTCSVLHEGLH |
| | | NHHTEKSLSHSPG |
| 87 | Pi-4212 | DIQMTQSPASLSASLGETVTIECLASEDIYSNLAWYQQKPGKSPQLLLYYANSLNDGVPSR |
| | light | FSVSGSGTQYSLKINSLQSEDVSLYFCQQHYDSPYTFGAGTKLELKRADAAPTVSLFPPSM |
| | chain | EQLTSGGATVVCFVNNFYPRDLSVKWKIDGSEQRDGVLDSVTDQDSKDSTYSMSSTLSLTK |
| | | VEYERHNLYTCEVVHKTSSSPVVKSFNRNEC |
| 88 | PI-9067s | ASTKGPSVFPLAPSSKSTSGGTAALGCLVKDYFPEPVTVSWNSGALTSGVHTFPAVLQSSG |
| | CH | LYSLSSVVTVPSSSLGTQTYICNVNHKPSNTKVDKKVEPKSCDKTHTCPPCPAPNLLGGPS |
| | | VFIFPPKIKDVLMISLSPIVTCVVVDVSEDDPDVQISWFVNNVEVHTAQTQTHREDYNSTL |
| | | RVVSALPIQHQDWMSGKEFKCKVNNKDLPAPIERTISKPKGSVRAPQVYVLPPPEEEMTKK |
| | | QVTLTCMVTDFMPEDIYVEWTNNGKTELNYKNTEPVLDSDGSYFMYSKLRVEKKNWVERNS |
| | | YSCSVVHEGLHNHHTTKSFSRTPGK |
| 89 | PI-9067s | RTVAAPSVFIFPPSDEQLKSGTASVVCLLNNFYPREAKVQWKVDNALQSGNSQESVTEQDS |
| | CL | KDSTYSLSSTLTLSKADYEKHKVYACEVTHQGLSSPVTKSFNRGEC |
| 90 | PI-9067L | AKTTAPSVYPLAPVCGDTTGSSVTLGCLVKGYFPEPVTLTWNSGSLSSGVHTFPAVLQSDL |
| | CH | YTLSSSVTVTSSTWPSQSITCNVAHPASSTKVDKKIEPRGPTIKPCPPCKCPAPNLLGGPS |
| | | VFIFPPKIKDVLMISLSPIVTCVVVDVSEDDPDVQISWFVNNVEVHTAQTQTHREDYNSTL |
| | | RVVSALPIQHQDWMSGKEFKCKVNNKDLPAPIERTISKPKGSVRAPQVYVLPPPEEEMTKK |
| | | QVTLTCMVTDFMPEDIYVEWTNNGKTELNYKNTEPVLDSDGSYFMYSKLRVEKKNWVERNS |
| | | YSCSVVHEGLHNHHTTKSFSRTPGK |
| 91 | PI-9067L | RADAAPTVSIFPPSSEQLTSGGASVVCFLNNFYPKDINVKWKIDGSERQNGVLNSWTDQDS |
| | CL | KDSTYSMSSTLTLTKDEYERHNSYTCEATHKTSTSPIVKSFNRNEC |
| 92 | PI-9069s | ASTKGPSVFPLAPSSKSTSGGTAALGCLVKDYFPEPVTVSWNSGALTSGVHTFPAVLQSSG |
| | CH | LYSLSSVVTVPSSSLGTQTYICNVNHKPSNTKVDKKVEPKSCDKTHTCPPCPAPNLLGGPS |
| | | VFIFPPKIKDVLMISLSPIVTCVVVDVSEDDPDVQISWFVNNVEVHTAQTQTHREDYNSTL |
| | | RVVSALPIQHQDWMSGKEFKCKVNNKDLPAPIERTISKPKGSVRAPQVYVLPPPEEEMTKK |
| | | QVTLTCMVTDFMPEDIYVEWTNNGKTELNYKNTEPVLDSDGSYFMYSKLRVEKKNWVERNS |
| | | YSCSVVHEGLHNHHTTKSFSRTPGK |
| 93 | PI-9069s | RTVAAPSVFIFPPSDEQLKSGTASVVCLLNNFYPREAKVQWKVDNALQSGNSQESVTEQDS |
| | CL | KDSTYSLSSTLTLSKADYEKHKVYACEVTHQGLSSPVTKSFNRGEC |
| 94 | PI-9069L | AKTTAPSVYPLAPVCGDTTGSSVTLGCLVKGYFPEPVTLTWNSGSLSSGVHTFPAVLQSDL |
| | CH | YTLSSSVTVTSSTWPSQSITCNVAHPASSTKVDKKIEPRGPTIKPCPPCKCPAPNLLGGPS |
| | | VFIFPPKIKDVLMISLSPIVTCVVVDVSEDDPDVQISWFVNNVEVHTAQTQTHREDYNSTL |
| | | RVVSALPIQHQDWMSGKEFKCKVNNKDLPAPIERTISKPKGSVRAPQVYVLPPPEEEMTKK |
| | | QVTLTCMVTDFMPEDIYVEWTNNGKTELNYKNTEPVLDSDGSYFMYSKLRVEKKNWVERNS |
| | | YSCSVVHEGLHNHHTTKSFSRTPGK |
| 95 | PI-9069L | RADAAPTVSIFPPSSEQLTSGGASVVCFLNNEYPKDINVKWKIDGSERQNGVLNSWTDQDS |
| | CL | KDSTYSMSSTLTLTKDEYERHNSYTCEATHKTSTSPIVKSFNRNEC |
| 96 | Mouse IgV | AIVLEEERYDLVEG |
| | domain 1, | |
| | residues | |
| | 21-34 | |
| 97 | Mouse IgV | LQVTDSG |
| | domain 1, | |
| | residues | |
| | 103-109 | |
| 98 | Mouse IgV | PVRLVVT |
| | domain 1, | |
| | residues | |
| | 128-134 | |
| 99 | Mouse IgV | QTLTVKCP |
| | domain 2, | |
| | residues | |
| | 35-42 | |
| 100 | Mouse IgV | PSEVHMGKFTLKHDPSEAMLQVQMTD |
| | domain 2, | |
| | residues | |
| | 77-102 | |
| 101 | Mouse IgV | FNIMKYANSQKAWQRLPDGKEPLTLVVTQRPFTR |
| | domain 3, | |
| | residues | |
| | 43-76 | |
| 102 | Mouse IgV | LYRCVIYHPPNDPVVLFH |
| | domain 3, | |
| | residues | |
| | 110-127 | |

1.-160. (canceled)

161. A method for increasing an immune response in a subject treated with an anti-Triggering Receptor Expressed on Myeloid Cells 1 (TREM1) antibody or antigen-binding fragment thereof, comprising administering to the subject an immunotherapy selected from the group consisting of: an immunotherapy that inhibits a checkpoint inhibitor; an immunotherapy that inhibits a checkpoint inhibitor of T cells; an anti-PD1 antibody; an anti-PDL1 antibody; an anti-CTLA4 antibody; pembrolizumab; nivolumab; and ipilimumab.

162. The method of claim 161, wherein the subject has cancer.

163. The method of claim 161, wherein the cancer is selected from the group consisting of: melanoma, kidney, hepatobiliary, head-neck squamous carcinoma, pancreatic, colon, bladder, glioblastoma, prostate, lung, and breast cancer.

164. The method of claim 161, wherein the immunotherapy is administered concurrently with the anti-TREM1 antibody or antigen-binding fragment thereof.

165. The method of claim 161, wherein the immunotherapy is administered subsequently to the anti-TREM1 antibody or antigen-binding fragment thereof.

166. The method of claim 161, wherein the subject has previously received an immunotherapy.

167. The method of claim 166, wherein the previous immunotherapy comprises at least one of an immunotherapy that inhibits a checkpoint inhibitor; an immunotherapy that inhibits a checkpoint inhibitor of T cells; an anti-PD1 antibody; an anti-PDL1 antibody; an anti-CTLA4 antibody; pembrolizumab; nivolumab; or ipilimumab.

168. The method of claim 161, wherein the anti-TREM1 antibody or antigen-binding fragment is at least one of a monoclonal antibody, an IgG1 antibody, an IgG4 antibody, an afucosylated antibody, a human antibody, a humanized antibody, a chimeric antibody, and a full length antibody.

169. The method of claim 161, wherein the anti-TREM1 antibody or antigen-binding fragment thereof comprises a human Fc domain.

170. The method of claim 161, wherein the anti-TREM1 antibody or antigen-binding fragment thereof comprises a human IgG1 Fc domain.

171. The method of claim 161, wherein the anti-TREM1 antibody or antigen-binding fragment thereof is an afucosylated antibody.

172. The method of claim 161, wherein the anti-TREM1 antibody or antigen-binding fragment thereof is a humanized antibody.

173. The method of claim 161, wherein the anti-TREM1 antibody or antigen-binding fragment thereof is present in an amount effective to kill, disable, or deplete a TREM1+ myeloid cell via antibody-dependent cell-mediated cytotoxicity activity, antibody-dependent phagocytosis activity, complement-dependent cytotoxicity activity, or antibody-mediated phagocytosis activity.

174. The method of claim 161, further comprising determining the expression level of TREM1 in a biological sample from the individual.

175. The method of claim 174, wherein the determining step comprises determining the presence of TREM1 mRNA expression or TREM1 protein expression.

176. The method of claim 174, wherein the determining step is carried out using at least one of FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, qPCR, RT-qPCR, multiplex qPCR or RT-qPCR, RNA-seq, microarray analysis, SAGE, MassARRAY technique, or FISH.

177. A method of identifying an individual who may respond to immunotherapy for the treatment of cancer comprising:
a. detecting the expression level of TREM1 in a biological sample from the individual; and
b. determining based on the expression level of TREM1, whether the individual may respond immunotherapy, wherein an elevated level of TREM1 in the individual relative to that in a healthy individual indicates that the individual may respond to immunotherapy.

178. The method of claim 177 wherein the immunotherapy comprises treatment with an anti-TREM1 antibody.

179. The method of claim 177 wherein the determining step comprises determining the presence of TREM1 mRNA expression or TREM1 protein expression.

180. A method of increasing an immune response in a subject, comprising administering an anti-TREM1 antibody comprising a) means for binding human TREM1 protein; and b) a human Fc domain.


SEQUENCE LISTING (102 sequences)

SEQ ID NO: 1 - protein, 117 aa, synthetic construct
  REGION 1..117: note = Description of Artificial Sequence: Synthetic polypeptide
EVQLVESGGGLVQPGGSLRLSCAASGFDIYSYSIHWVRQAPGKGLEWVAYIYPSYGYTSYADSVKGRFTISADTSKNTAYLQMNSLRAEDTAVYYCARGAVAALDYWGQGTLVTVSS

SEQ ID NO: 2 - protein, 110 aa, synthetic construct
  REGION 1..110: note = Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYGVSGYSLITFGQGTKVEIK

SEQ ID NO: 3 - protein, 115 aa, synthetic construct
  REGION 1..115: note = Description of Artificial Sequence: Synthetic polypeptide
EVQLVESGGGLVQPGGSLRLSCAASGFDISSSSMHWVRQAPGKGLEWVASISSSYSYTYYADSVKGRFTISADTSKNTAYLQMNSLRAEDTAVYYCARYVAMDYWGQGTLVTVSS

SEQ ID NO: 4 - protein, 109 aa, synthetic construct
  REGION 1..109: note = Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQWGGGHYLFTFGQGTKVEIK

SEQ ID NO: 5 - protein, 120 aa, synthetic construct
  REGION 1..120: note = Description of Artificial Sequence: Synthetic polypeptide
EVQLVESGGGLVQPGGSLRLSCAASGFDLYSSYIHWVRQAPGKGLEWVASIYPSYGYTYYADSVKGRFTISADTSKNTAYLQMNSLRAEDTAVYYCARSHSYVHGAMDYWGQGTLVTVSS

SEQ ID NO: 6 - protein, 110 aa, synthetic construct
  REGION 1..110: note = Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYWWVPAAPITFGQGTKVEIK

SEQ ID NO: 7 - protein, 6 aa, synthetic construct
  REGION 1..6: note = Description of Artificial Sequence: Synthetic peptide
IYSYSI

SEQ ID NO: 8 - protein, 10 aa, synthetic construct
  REGION 1..10: note = Description of Artificial Sequence: Synthetic peptide
YIYPSYGYTS

SEQ ID NO: 9 - protein, 6 aa, synthetic construct
  REGION 1..6: note = Description of Artificial Sequence: Synthetic peptide
GAVAAL

SEQ ID NO: 10 - protein, 5 aa, synthetic construct
  REGION 1..5: note = Description of Artificial Sequence: Synthetic peptide
SVSSA

SEQ ID NO: 11 - protein, 7 aa, synthetic construct
  REGION 1..7: note = Description of Artificial Sequence: Synthetic peptide
SASSLYS

SEQ ID NO: 12 - protein, 9 aa, synthetic construct
  REGION 1..9: note = Description of Artificial Sequence: Synthetic peptide
YGVSGYSLI

SEQ ID NO: 13 - protein, 6 aa, synthetic construct
  REGION 1..6: note = Description of Artificial Sequence: Synthetic peptide
ISSSSM

SEQ ID NO: 14 - protein, 10 aa, synthetic construct
  REGION 1..10: note = Description of Artificial Sequence: Synthetic peptide
SISSSYSYTY

SEQ ID NO: 15 - protein, 4 aa, synthetic construct
  REGION 1..4: note = Description of Artificial Sequence: Synthetic peptide
YVAM

SEQ ID NO: 16 - protein, 5 aa, synthetic construct
  REGION 1..5: note = Description of Artificial Sequence: Synthetic peptide
SVSSA

SEQ ID NO: 17 - protein, 7 aa, synthetic construct
  REGION 1..7: note = Description of Artificial Sequence: Synthetic peptide
SASSLYS

SEQ ID NO: 18 - protein, 8 aa, synthetic construct
  REGION 1..8: note = Description of Artificial Sequence: Synthetic peptide
WGGGHYLF

SEQ ID NO: 19 - protein, 6 aa, synthetic construct
  REGION 1..6: note = Description of Artificial Sequence: Synthetic peptide
LYSSYI

SEQ ID NO: 20 - protein, 10 aa, synthetic construct
  REGION 1..10: note = Description of Artificial Sequence: Synthetic peptide
SIYPSYGYTY

SEQ ID NO: 21 - protein, 9 aa, synthetic construct
  REGION 1..9: note = Description of Artificial Sequence: Synthetic peptide
SHSYVHGAM

SEQ ID NO: 22 - protein, 5 aa, synthetic construct
  REGION 1..5: note = Description of Artificial Sequence: Synthetic peptide
SVSSA

SEQ ID NO: 23 - protein, 7 aa, synthetic construct
  REGION 1..7: note = Description of Artificial Sequence: Synthetic peptide
SASSLYS

SEQ ID NO: 24 - protein, 9 aa, synthetic construct
  REGION 1..9: note = Description of Artificial Sequence: Synthetic peptide
YWWVPAAPI

SEQ ID NO: 25 - protein, 121 aa, synthetic construct
  REGION 1..121: note = Description of Artificial Sequence: Synthetic polypeptide
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSS

SEQ ID NO: 26 - protein, 111 aa, synthetic construct
  REGION 1..111: note = Description of Artificial Sequence: Synthetic polypeptide
DIVLTQSPDSLAVSLGERATINCRASESVDTFDYSFLHWYQQKPGQPPKLLIYRASNLESGVPDRFSGSGSGTDFTLTISSLQAEDVAVYYCQQSNEDPYTFGQGTKLEIK

SEQ ID NO: 27 - protein, 128 aa, synthetic construct
  REGION 1..128: note = Description of Artificial Sequence: Synthetic polypeptide
EVQLVESGGGLVQPGGSLRLSCAASGFDISYSSIHWVRQAPGKGLEWVASIYSSYGSTYYADSVKGRFTISADTSKNTAYLQMNSLRAEDTAVYYCARYYYSHAGWYVSGYWPAIDYWGQGTLVTVSS

SEQ ID NO: 28 - protein, 107 aa, synthetic construct
  REGION 1..107: note = Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSSYSLITFGQGTKVEIK

SEQ ID NO: 29 - protein, 121 aa, synthetic construct
  REGION 1..121: note = Description of Artificial Sequence: Synthetic polypeptide
EVQLVESGGGLVQPGGSLRLSCAASGFDLYSSSIHWVRQAPGKGLEWVASISPYYSSTSYADSVKGRFTISADTSKNTAYLQMNSLRAEDTAVYYCARSVYYTVYFGLDYWGQGTLVTVSS

SEQ ID NO: 30 - protein, 110 aa, synthetic construct
  REGION 1..110: note = Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQAVGAWGYLITFGQGTKVEIK

SEQ ID NO: 31 - protein, 5 aa, synthetic construct
  REGION 1..5: note = Description of Artificial Sequence: Synthetic peptide
TYAMH

SEQ ID NO: 32 - protein, 19 aa, synthetic construct
  REGION 1..19: note = Description of Artificial Sequence: Synthetic peptide
RIRTKSSNYATYYAASVKG

SEQ ID NO: 33 - protein, 10 aa, synthetic construct
  REGION 1..10: note = Description of Artificial Sequence: Synthetic peptide
DMGIRRQFAY

SEQ ID NO: 34 - protein, 15 aa, synthetic construct
  REGION 1..15: note = Description of Artificial Sequence: Synthetic peptide
RASESVDTFDYSFLH

SEQ ID NO: 35 - protein, 7 aa, synthetic construct
  REGION 1..7: note = Description of Artificial Sequence: Synthetic peptide
RASNLES

SEQ ID NO: 36 - protein, 9 aa, synthetic construct
  REGION 1..9: note = Description of Artificial Sequence: Synthetic peptide
QQSNEDPYT

SEQ ID NO: 37 - protein, 6 aa, synthetic construct
  REGION 1..6: note = Description of Artificial Sequence: Synthetic peptide
ISYSSI

SEQ ID NO: 38 - protein, 10 aa, synthetic construct
  REGION 1..10: note = Description of Artificial Sequence: Synthetic peptide
SIYSSYGSTY

SEQ ID NO: 39 - protein, 17 aa, synthetic construct
  REGION 1..17: note = Description of Artificial Sequence: Synthetic peptide
YYYSHAGWYVSGYWPAI

SEQ ID NO: 40 - protein, 5 aa, synthetic construct
  REGION 1..5: note = Description of Artificial Sequence: Synthetic peptide
SVSSA

SEQ ID NO: 41 - protein, 7 aa, synthetic construct
  REGION 1..7: note = Description of Artificial Sequence: Synthetic peptide
SASSLYS

SEQ ID NO: 42 - protein, 6 aa, synthetic construct
  REGION 1..6: note = Description of Artificial Sequence: Synthetic peptide
SSYSLI

SEQ ID NO: 43 - protein, 6 aa, synthetic construct
  REGION 1..6: note = Description of Artificial Sequence: Synthetic peptide
LYSSSI

SEQ ID NO: 44 - protein, 10 aa, synthetic construct
  REGION 1..10: note = Description of Artificial Sequence: Synthetic peptide
SISPYYSSTS

SEQ ID NO: 45 - protein, 10 aa, synthetic construct
  REGION 1..10: note = Description of Artificial Sequence: Synthetic peptide
SVYYTVYFGL

SEQ ID NO: 46 - protein, 5 aa, synthetic construct
  REGION 1..5: note = Description of Artificial Sequence: Synthetic peptide
SVSSA

SEQ ID NO: 47 - protein, 7 aa, synthetic construct
  REGION 1..7: note = Description of Artificial Sequence: Synthetic peptide
SASSLYS

SEQ ID NO: 48 - protein, 9 aa, synthetic construct
  REGION 1..9: note = Description of Artificial Sequence: Synthetic peptide
AVGAWGYLI

SEQ ID NO: 49 - protein, 123 aa, synthetic construct
  REGION 1..123: note = Description of Artificial Sequence: Synthetic polypeptide
EVQLVESGGGLVQPGGSLRLSCAASGFDLSYSSMHWVRQAPGKGLEWVAYISPYSGYTSYADSVKGRFTISADTSKNTAYLQMNSLRAEDTAVYYCARHGSWSYGFSSGFDYWGQGTLVTVSS

SEQ ID NO: 50 - protein, 108 aa, synthetic construct
  REGION 1..108: note = Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYAYYYPITFGQGTKVEIK

SEQ ID NO: 51 - protein, 116 aa, synthetic construct
  REGION 1..116: note = Description of Artificial Sequence: Synthetic polypeptide
QVQLKESGPGLVQPSQTLSLTCTVSGFSITSSNVYWFRQPPGKGLEWMGNIWGDGSTDYNSSLKSRLTLSRDTSKNQVFLKINSLQSEDTATYFCTRSWEYYFDHWGQGVVVTVSS

SEQ ID NO: 52 - protein, 110 aa, synthetic construct
  REGION 1..110: note = Description of Artificial Sequence: Synthetic polypeptide
DIVLTQSPALAVSPGQRATLSCRASQSVTLSNVNLMNWYQQKPGQQPKLLIYHASNLASGIPTRFSGSGSGTDFTLTIDPVQADDIAAYYCQQSGESPRTFGGGTKVELK

SEQ ID NO: 53 - protein, 119 aa, synthetic construct
  REGION 1..119: note = Description of Artificial Sequence: Synthetic polypeptide
QVQLQQSGAELVKPGSSVKLSCKASGYTFTSYDMHWIKQQPGDGLEWLGWLYPGNGNSKYNQKFDGKATLTADKSSSTAYLQLSSLTSEDSAVYFCARRGEFTYYFDYWGQGVMVTVSS

SEQ ID NO: 54 - protein, 107 aa, synthetic construct
  REGION 1..107: note = Description of Artificial Sequence: Synthetic polypeptide
DIQMTQSPASLSASLGETVTIECLASEDIYSNLAWYQQKPGKSPQLLLYYANSLNDGVPSRFSVSGSGTQYSLKINSLQSEDVSLYFCQQHYDSPYTFGAGTKLELK

SEQ ID NO: 55 - protein, 6 aa, synthetic construct
  REGION 1..6: note = Description of Artificial Sequence: Synthetic peptide
LSYSSM

SEQ ID NO: 56 - protein, 10 aa, synthetic construct
  REGION 1..10: note = Description of Artificial Sequence: Synthetic peptide
YISPYSGYTS

SEQ ID NO: 57 - protein, 12 aa, synthetic construct
  REGION 1..12: note = Description of Artificial Sequence: Synthetic peptide
HGSWSYGFSSGF

SEQ ID NO: 58 - protein, 5 aa, synthetic construct
  REGION 1..5: note = Description of Artificial Sequence: Synthetic peptide
SVSSA

SEQ ID NO: 59 - protein, 7 aa, synthetic construct
  REGION 1..7: note = Description of Artificial Sequence: Synthetic peptide
SASSLYS

SEQ ID NO: 60 - protein, 7 aa, synthetic construct
  REGION 1..7: note = Description of Artificial Sequence: Synthetic peptide
YAYYYPI

SEQ ID NO: 61 - protein, 10 aa, synthetic construct
  REGION 1..10: note = Description of Artificial Sequence: Synthetic peptide
GFSITSSNVY

SEQ ID NO: 62 - protein, 16 aa, synthetic construct
  REGION 1..16: note = Description of Artificial Sequence: Synthetic peptide
NIWGDGSTDYNSSLKS

SEQ ID NO: 63 - protein, 8 aa, synthetic construct
  REGION 1..8: note = Description of Artificial Sequence: Synthetic peptide
SWEYYFDH

SEQ ID NO: 64 - protein, 15 aa, synthetic construct
  REGION 1..15: note = Description of Artificial Sequence: Synthetic peptide
RASQSVTLSNVNLMN

SEQ ID NO: 65 - protein, 7 aa, synthetic construct
  REGION 1..7: note = Description of Artificial Sequence: Synthetic peptide
HASNLAS

SEQ ID NO: 66 - protein, 9 aa, synthetic construct
  REGION 1..9: note = Description of Artificial Sequence: Synthetic peptide
QQSGESPRT

SEQ ID NO: 67 - protein, 10 aa, synthetic construct
  REGION 1..10: note = Description of Artificial Sequence: Synthetic peptide
GYTFTSYDMH

SEQ ID NO: 68 - protein, 17 aa, synthetic construct
  REGION 1..17: note = Description of Artificial Sequence: Synthetic peptide
WLYPGNGNSKYNQKFDG

SEQ ID NO: 69 - protein, 10 aa, synthetic construct
  REGION 1..10: note = Description of Artificial Sequence: Synthetic peptide
RGEFTYYFDY

SEQ ID NO: 70 - protein, 11 aa, synthetic construct
  REGION 1..11: note = Description of Artificial Sequence: Synthetic peptide
LASEDIYSNLA

SEQ ID NO: 71 - protein, 7 aa, synthetic construct
  REGION 1..7: note = Description of Artificial Sequence: Synthetic peptide
YANSLND

SEQ ID NO: 72 - protein, 9 aa, synthetic construct
  REGION 1..9: note = Description of Artificial Sequence: Synthetic peptide
QQHYDSPYT

SEQ ID NO: 73 - protein, 5 aa, synthetic construct
  REGION 1..5: note = Description of Artificial Sequence: Synthetic polypeptide
  REGION 1..5: note = The entire sequence of amino acids 1-5 can be repeated one to six times.
GGGGS

SEQ ID NO: 74 - protein, 447 aa, synthetic construct
  REGION 1..447: note = Description of Artificial Sequence: Synthetic polypeptide
EVQLVESGGGLVQPGGSLRLSCAASGFDIYSYSIHWVRQAPGKGLEWVAYIYPSYGYTSYADSVKGRFTISADTSKNTAYLQMNSLRAEDTAVYYCARGAVAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 75 - protein, 217 aa, synthetic construct
  REGION 1..217: note = Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYGVSGYSLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 447 aa, synthetic construct
  REGION 1..447: note = Description of Artificial Sequence: Synthetic polypeptide
EVQLVESGGGLVQPGGSLRLSCAASGFDIYSYSIHWVRQAPGKGLEWVAYIYPSYGYTSYADSVKGRFTISADTSKNTAYLQMNSLRAEDTAVYYCARGAVAALDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 77 - protein, 217 aa, synthetic construct
  REGION 1..217: note = Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYGVSGYSLITFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 78 - protein, 217 aa, synthetic construct
  REGION 1..217: note = Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYWWVPAAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 217 aa, synthetic construct; identical to SEQ ID NO: 78
  REGION 1..217: note = Description of Artificial Sequence: Synthetic polypeptide

SEQ ID NO: 80 - protein, 450 aa, synthetic construct
  REGION 1..450: note = Description of Artificial Sequence: Synthetic polypeptide
EVQLVESGGGLVQPGGSLRLSCAASGFDLYSSYIHWVRQAPGKGLEWVASIYPSYGYTYYADSVKGRFTISADTSKNTAYLQMNSLRAEDTAVYYCARSHSYVHGAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 81 - protein, 217 aa, synthetic construct
  REGION 1..217: note = Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYWWVPAAPITFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 82 - protein, 453 aa, synthetic construct
  REGION 1..453: note = Description of Artificial Sequence: Synthetic polypeptide
EVQLVESGGGLVQPGGSLRLSCAASGFDLSYSSMHWVRQAPGKGLEWVAYISPYSGYTSYADSVKGRFTISADTSKNTAYLQMNSLRAEDTAVYYCARHGSWSYGFSSGFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 83 - protein, 215 aa, synthetic construct
  REGION 1..215: note = Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYAYYYPITFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 84 - protein, 446 aa, synthetic construct
  REGION 1..446: note = Description of Artificial Sequence: Synthetic polypeptide
QVQLKESGPGLVQPSQTLSLTCTVSGFSITSSNVYWFRQPPGKGLEWMGNIWGDGSTDYNSSLKSRLTLSRDTSKNQVFLKINSLQSEDTATYFCTRSWEYYFDHWGQGVVVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 85 - protein, 217 aa, synthetic construct
  REGION 1..217: note = Description of Artificial Sequence: Synthetic polypeptide
DIVLTQSPALAVSPGQRATLSCRASQSVTLSNVNLMNWYQQKPGQQPKLLIYHASNLASGIPTRFSGSGSGTDFTLTIDPVQADDIAAYYCQQSGESPRTFGGGTKVELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 86 - protein, 440 aa, synthetic construct
  REGION 1..440: note = Description of Artificial Sequence: Synthetic polypeptide
QVQLQQSGAELVKPGSSVKLSCKASGYTFTSYDMHWIKQQPGDGLEWLGWLYPGNGNSKYNQKFDGKATLTADKSSSTAYLQLSSLTSEDSAVYFCARRGEFTYYFDYWGQGVMVTVSSTETTAPSVYPLAPGTALKSNSMVTLGCLVKGYFPEPVTVTWNSGALSSGVHTFPAVLQSGLYTLTSSVTVPSSTWSSQAVTCNVAHPASSTKVDKKIVPRECNPCGCTGSEVSSVFLFPPKTKDVLTLTLTPKVTCVVVDISQNDPEVRFSWFLDDVEVHTAQTHAPEKQSNSTLRSVSELPIVHRDWLDGKTFKCKVNSGAFPAPIEKSLSKPEGTPRGPQVYTMAPPKEEMTQSQVSITCMVKGFYPPDLYTEWKMNGQPQENYKNTPPTMDTDGSYFLYSKLNVKKETWQQGNTFTCSVLHEGLHNHHTEKSLSHSPG

SEQ ID NO: 87 - protein, 214 aa, synthetic construct
  REGION 1..214: note = Description of Artificial Sequence: Synthetic polypeptide
DIQMTQSPASLSASLGETVTIECLASEDIYSNLAWYQQKPGKSPQLLLYYANSLNDGVPSRFSVSGSGTQYSLKINSLQSEDVSLYFCQQHYDSPYTFGAGTKLELKRADAAPTVSLFPPSMEQLTSGGATVVCFVNNFYPRDLSVKWKIDGSEQRDGVLDSVTDQDSKDSTYSMSSTLSLTKVEYERHNLYTCEVVHKTSSSPVVKSFNRNEC

SEQ ID NO: 88 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 89 - protein, 107 aa, synthetic construct [sequence omitted]

SEQ ID NO: 90 - protein, 330 aa, synthetic construct
  REGION 1..330: note = Description of Artificial Sequence: Synthetic polypeptide
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 91 - protein, 107 aa, synthetic construct
  REGION 1..107: note = Description of Artificial Sequence: Synthetic polypeptide
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 92 - protein, 330 aa, synthetic construct; identical to SEQ ID NO: 88
  REGION 1..330: note = Description of Artificial Sequence: Synthetic polypeptide

SEQ ID NO: 93 - protein, 107 aa, synthetic construct [sequence omitted]

SEQ ID NO: 94 - protein, 330 aa, synthetic construct; identical to SEQ ID NO: 90
  REGION 1..330: note = Description of Artificial Sequence: Synthetic polypeptide

SEQ ID NO: 95 - protein, 107 aa, synthetic construct; identical to SEQ ID NO: 91
  REGION 1..107: note = Description of Artificial Sequence: Synthetic polypeptide

SEQ ID NO: 96 - protein, 14 aa, Mus musculus
AIVLEEERYDLVEG

SEQ ID NO: 97 - protein, 7 aa, Mus musculus
LQVTDSG

SEQ ID NO: 98 - protein, 7 aa, Mus musculus
PVRLVVT

SEQ ID NO: 99 - protein, 8 aa, Mus musculus
QTLTVKCP

SEQ ID NO: 100 - protein, 26 aa, Mus musculus
PSEVHMGKFTLKHDPSEAMLQVQMTD

SEQ ID NO: 101 - protein, 34 aa, Mus musculus
FNIMKYANSQKAWQRLPDGKEPLTLVVTQRPFTR

SEQ ID NO: 102 - protein, 18 aa, Mus musculus
LYRCVIYHPPNDPVVLFH